(19) United States
(12) Patent Application Publication
(10) Pub. No.: US 2009/0047710 A1
(43) Pub. Date: Feb. 19, 2009

(54) ST3Gal-1/ST6GalNAc-1 Chimeras

(75) Inventor: Shawn DeFrees, North Wales, PA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Neose Technologies, Inc., Horsham, PA (US)

(21) Appl. No.: 12/093,013

(22) PCT Filed: Nov. 8, 2006
(86) PCT No.: PCT/US06/43657

§ 371 Date: Jun. 2, 2008

Related U.S. Application Data
(60) Provisional application No. 60/735,922, filed on Nov. 9, 2005.

(51) Int. Cl. C12P 21/00; C12N 9/10; C07H 21/00; C12N 15/63; C12N 1/00; C12N 1/21

(52) U.S. Cl. 435/69.1; 435/193; 536/23.2; 435/243; 435/252.33; 435/252.34; 435/252.31; 435/252.3

(57) ABSTRACT

The present invention features compositions and methods related to increasing the solubility and enzymatic activity of GalNAc-α-2,6-sialyltransferase I (STÌGalNAcI) proteins expressed in prokaryotic host cells. Methods for increasing the solubility of STÌGalNAcI polypeptides include modifying cysteine residues, modifying N-linked glycosylation sites, deleting polypeptide regions, and constructing chimeric polypeptides comprising sequences from a STÌGalNAcI and another protein, for example, a Gal-β-1,3GalNAc-α-2,3-sialyltransferase (ST3GalI) and a STÌGalNAcI. The invention also features nucleic acids encoding such improved polypeptides, as well as vectors, host cells, expression systems, and methods of expressing and using such polypeptides.

### CROSS REFERENCES TO RELATED APPLICATIONS

This application claims the benefit of U.S. Provisional Application No. 60/735,922, filed on Nov. 9, 2005, the disclosure of which is hereby incorporated herein by reference in its entirety for all purposes.

### FIELD OF INVENTION

The present invention features compositions and methods related to increasing the solubility and enzymatic activity of GalNAc-α-2,6-sialyltransferase I (ST6GalNAcI) proteins expressed in prokaryotic host cells. The invention also features nucleic acids encoding such improved polyeptides, as well as vectors, host cells, expression systems, and methods of expressing and using such polypeptides.

### BACKGROUND OF THE INVENTION

A great diversity of oligosaccharide structures and many types of glycopeptides are found in nature, and these are synthesized, in part, by a large number of glycosyltransferases. Glycosyltransferases catalyze the synthesis of glycolipids, glycopeptides, and polysaccharides, by transferring an activated mono- or oligosaccharide residue to an existing acceptor molecule for the initiation or elongation of the carbohydrate chain. A catalytic reaction is believed to involve the recognition of both the donor and acceptor by suitable domains, as well as the catalytic site of the enzyme.

Many peptide therapeutics, and many potential peptide therapeutics, are glycosylated peptides. The production of a recombinant glycopeptide, as opposed to a recombinant non-glycosylated peptide, requires that a recombinantly-produced peptide is subjected to additional processing steps, either within the cell or after the peptide is produced by the cell, where the processing steps are performed in vitro. The peptide can be treated enzymatically to introduce one or more glycosyl groups onto the peptide, using a glycosyltransferase. Specifically, the glycosyltransferase covalently attaches the glycosyl group or groups to the peptide.

The extra in vitro steps of peptide processing to produce a glycopeptide can be time consuming and costly. This is due, in part, to the burden and cost of producing recombinant glycosyltransferases for the in vitro glycosylation of peptides and glycopeptides to produce glycopeptide therapeutics. As the demand and usefulness of recombinant glycotherapeutics increases, new methods are required in order to more efficiently prepare glycopeptides. Moreover, as more and more glycopeptides are discovered to be useful for the treatment of a variety of diseases, there is a need for methods that lower the cost of their production. Further, there is also a need in the art to develop methods of more efficiently producing recombinant glycopeptides for use in developing and improving glycopeptide therapeutics.

Glycosyltransferases are reviewed in general in International (PCT) Patent Application No. WO 03/031464 (PCT/US02/32263), which is incorporated herein by reference in its entirety. One such particular glycosyltransferase that has utility in the development and production of therapeutic glycopeptides is ST6GalNAcI. ST6GalNAcI, or GalNAcα-2,6-sialyltransferase, catalyzes the transfer of sialic acid from a sialic acid donor to a sialic acid acceptor. Full length chicken ST6GalNAcI enzyme, for example, is disclosed by Kurosawa et al. (1994, J. Biol. Chem. 269:1402-1409). However, the identification of useful mutants of this enzyme, having enhanced biological activity such as enhanced catalytic activity, stability or solubility, has not heretofore been reported.

In the past, there have been efforts to increase the availability of recombinant glycosyltransferases for the in vitro production of glycopeptides. To date, a limited amount of work has been done with respect to recombinant glycosyltransferases that may sometimes be suitable for small-scale production of oligosaccharides or glycopeptides. For example, Kurosawa et al. (1994, J Biol. Chem. 269:1402-1409) describe a truncation mutant of chicken ST6GalNAcI lacking amino acid residues 1-232 of the full-length enzyme. A truncation of mouse ST6GalNAcI was also reported by Kurosawa et al. (2000, J. Biochem., 127:845-854). However, for example, the truncated chicken enzyme described by Kurosawa et al. lacks the substrate specificity of other ST6GalNAcI enzymes and lacks the activity required for “pharmaceutical-scale” processes and reactions, including the production of glycopeptide therapeutics. Additional examples of truncated and modified ST6GalNAcI polypeptides are described in International Application No. PCT/US05/19583, the disclosure of which is hereby incorporated herein by reference in its entirety.

Therefore, a need still exists for recombinant glycosyltransferases having activity that is suitable for “pharmaceutical-scale” processes and reactions, including the production of glycopeptide therapeutics. In particular, there is a need for recombinant glycosyltranasferases having favorable functional and structural characteristics. Further, a need exists for efficient methods of identification and characterization of recombinant glycosyltransferases, as well as for the production of such glycosyltransferases. The present invention addresses and meets these needs.

### BRIEF SUMMARY OF THE INVENTION

The present invention provides compositions and methods for enhanced solubility and enzymatic activity of ST6GalNAcI polypeptides expressed in prokaryotic host cells. The improved ST6GalNAcI nucleic acid and amino acid sequences are modified in one or more of a number of ways, including construction of chimeras with another protein sequence, for example, a ST3 GalI sequence, modification of one or more cysteine residues or one or more N-linked glycosylation sites, truncation of one or more amino acids from select regions, including N-terminal regions, including a signal peptide domain, a transmembrane domain, and a stem domain.

Accordingly, in a first aspect, the invention provides chimeric polypeptides comprising a first portion from a Gal-β-1,3GalNAc-α-2,3-sialyltransferase (ST3GalI) polypeptide and a second portion from a GalNAc-α-2,6-sialyltransferase I (ST6GalNAcI) polypeptide, wherein the polypeptide has ST6GalNAcI transferase activity. The portions can be contiguous or non-contiguous.

In some embodiments, a chimera will comprise an N-terminal region from a ST3GalI polypeptide and a C-terminal region from a ST6GalNAcI polypeptide. In some embodiments, internal sequence segments of a ST6GalNAcI polypeptide are replaced with corresponding internal sequence segments of a ST3GalI polypeptide. Exemplifed polypeptide chimeras are shown in FIGS. 2A-E. Chimeric polypeptides of the invention can share at least 90%, 91%, 92%, 93%, 94%, 95%, 96%, 97%, 98%, 99% sequence identity with the exemplified sequences shown in FIGS. 2A-E.

In other embodiments, a chimera will comprise an N-terminal region from a ST6GalNAcI polypeptide and a C-terminal region from a ST3Gal polypeptide, wherein the polypeptide catalyzes the transfer of a sialic acid moiety from a donor moiety to an acceptor moiety. Such a chimera preferably will maintain ST6GalNAc transferase activity. This chimera is exemplified in FIG. 2E.

In some embodiments, a modified ST6GalNAcI or a ST3GalI/ST6GalNAcI chimera is truncated at the N-terminal region. One or more amino acids from the signal peptide, the transmembrane domain, and/or the stem domain can be eliminated. In some embodiments, a modified ST6GalNAcI or a ST3GalI/ST6GalNAcI chimera will have one or more modified cysteine residues or one or more modified N-linked glycosylation sites. For example, one or more non-native N-linked glycosylation sites or cysteine residues can be introduced, or one or more native N-linked glycosylation sites or cysteine residues can be eliminated. An exemplifed modified ST6GalNAcI sequence having one or modified N-linked glycosylation sites is shown in FIG. 2E-F (ST6GalNAcI N-linked glycosylation mutants).

The polypeptides can optionally have a tag, for example a purification tag or detection tag. Exemplified tags include a maltose binding protein (MBP) tag, a histidine tag, a Factor IX tag, a glutathion-5-transferase (GST) tag, a FLAG-tag, and a starch binding domain (SBD) tag.

The improved polypeptides of the invention are modified in comparison to native ST6GalNAcI polypeptides or native ST3GalI polypeptides from animal species including human, chimpanzee, orangutan, pig, cow, dog, rat, mouse and chicken.

The polypeptides of the invention can preferentially express as active enzyme in the soluble fraction of a prokaryotic host cell or can be easily purified and refolded from inclusion bodies.

In a further aspect, the invention provides nucleic acids encoding the polypeptides of the invention, and expression cassettes, expression vectors and prokaryotic host cells containing the modified ST6GalNAcI nucleic acids and/or polypeptides of the invention. Exemplified prokaryotic host cells include bacillales (Bacillus) and proteobacteria, including alphaproteobacteria (e.g., Caulobacterales), betaproteobacteria (e.g., Burkholderialies, including Ralstonia), and gammaproteobacteria (e.g., Pseudomonadales (Pseudomonas fluorescens group) and Enterobacteriales (Escherichia coli).

In another aspect, the invention provides methods of producing a modified ST6GalNAcI polypeptide, the method comprising growing a recombinant prokaryotic host cell under conditions suitable for expression of the modified ST6GalNAcI polypeptide.

In a another aspect, the invention provides methods of catalyzing the transfer of a sialic acid moiety to an acceptor moiety comprising incubating the modified ST6GalNAcI polypeptide of the invention with a sialic acid moiety and an acceptor moiety, wherein the polypeptide mediates the covalent linkage of the sialic acid moiety to the acceptor moiety, thereby catalyzing the transfer of a sialic acid moiety to an acceptor moiety.

In a further aspect, the invention provides methods catalyzing the transfer of a sialic acid moiety to an acceptor moiety comprising incubating a modified ST6GalNAcI polypeptide of the invention with a cytidinemonophosphate-sialic acid (CMP-NAN) sialic acid donor and an asialo bovine submaxillary mucin acceptor moiety, wherein the polypeptide mediates the transfer of the sialic acid moiety from the CMP-NAN sialic acid donor to the asialo bovine submaxillary mucin acceptor, thereby catalyzing the transfer of a sialic acid moiety to an acceptor moiety.

### BRIEF DESCRIPTION OF THE DRAWINGS

For purpose of illustrating the invention, there are depicted in the drawings certain embodiments of the invention. However, the invention is not limited to the precise arrangements and instrumentalities of the embodiments depicted in the drawings.

FIG. 1 illustrates an alignment of a porcine ST3GalI amino acid sequence (GenBank Accession Nos. AAA31125; Q02745 or NP_{—}001004047; SEQ ID NO:25) with a human ST6GalNAcI amino acid sequence (GenBank Accession Nos. CAA72179 or Q9NSC7; SEQ ID NO:26).

FIG. 2 illustrates exemplified amino acid sequences of ST6GalNAc polypeptides modified for enhanced solubility and/or enzymatic activity. Modifications can include truncations of one or more amino acid residues from certain regions, for example N-terminal regions, including the signal peptide domain, the transmembrane domain, and the stem domain. Other modifications can include modifying one or more cysteine residues or N-linked glycosylation sites in the native sequence. Modifications also include constructing chimeras of ST6GalNAcI polypeptides sequences and ST3 GalI polypeptide sequences.

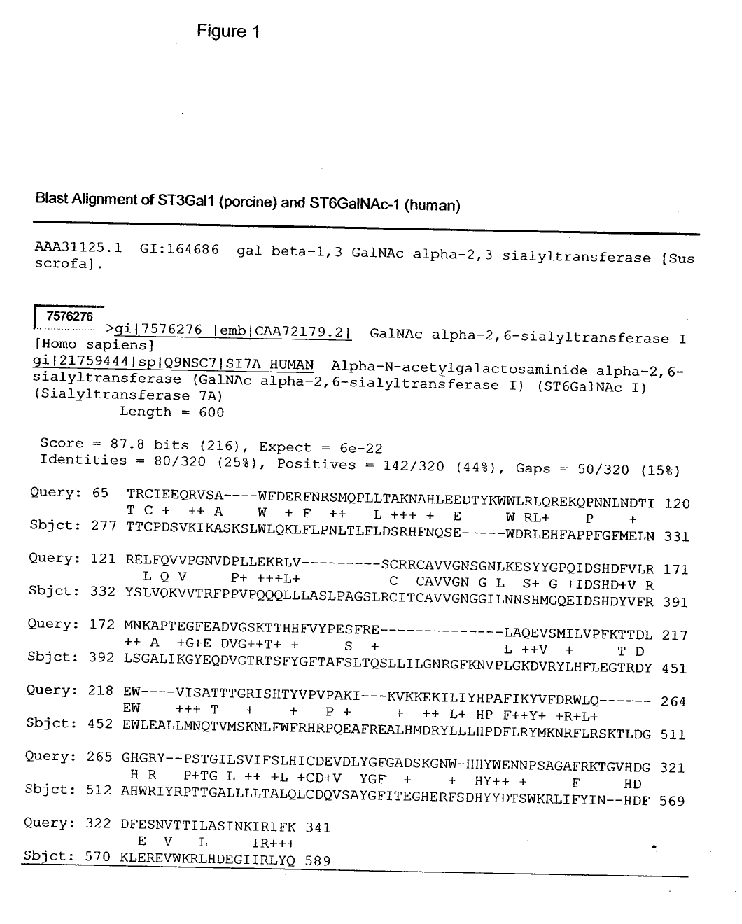

### DETAILED DESCRIPTION OF THE INVENTION

The compositions and methods of the present invention encompass improved ST6GalNAcI nucleic acid and amino acid sequences modified for increased solubility and/or enzymatic activity in a prokaryotic host. The modification can include one or more of constructing a chimera of ST6GalNAcI sequences and ST3GalI sequences, modifying (e.g., deleting, adding or changing) one or more cysteine residues, modifying one or more N-linked glycosylation sites, and/or eliminating selected sequence segments, including truncation of one or more amino acids from N-terminal regions, including the signal peptide domain, the transmembrane domain and the stem domain. As stated above, exemplified truncated and modified ST6GalNAcI polypeptides are described in International Application Publication No. WO 2005/121332, the disclosures of each of which is hereby incorporated herein by reference in its entirety.

The glycosyltransferase ST6GalNAcI is an essential reagent for glycosylation of therapeutic glycopeptides. Additionally, ST6GalNAcI is an important reagent for research and development of therapeutically important glycopeptides and oligosaccharide therapeutics. ST6GalNAcI is typically isolated and purified from natural sources, or from tedious and costly in vitro and recombinant sources. The present invention provides compositions and methods relating to simplified and more cost-effective methods of production of ST6GalNAcI enzymes. In particular, the present invention provides compositions and methods relating to modified ST6GalNAcI enzymes that have improved and useful properties in comparison to their native or wild-type enzyme counterparts.

The modified ST6GalNAcI glycosyltransferase enzymes of the present invention are useful for in vivo and in vitro preparation of glycosylated peptides, as well as for the production of oligosaccharides containing the specific glycosyl residues that can be transferred by the modified glycosyltransferase enzymes of the present invention. Modified forms of ST6GalNAcI polypeptides can possess biological activities comparable to, and in some instances, in excess of their full-length polypeptide counterparts. The present application also discloses that modified ST6GalNAcI polypeptides not only possess biological activity, but also can have enhanced properties of solubility, stability and resistance to proteolytic degradation.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described herein.

Certain abbreviations are used herein as are common in the art, such as: “Ac” for acetyl; “Glc” for glucose; “Glc” for glucosamine; “GlcA for glucuronic acid; “IdoA” for iduronic acid; “GlcNAc” for N-acetylglucosamine; “NAN” or “sialic acid” or “SA” for N-acetyl neuraminic acid; “UDP” for uridine diphosphate; “CMP” for cytidine monophosphate.

An “affinity tag” is a peptide or polypeptide that may be genetically or chemically fused to a second polypeptide for the purposes of purification, isolation, targeting, trafficking, or identification of the second polypeptide. The “genetic” attachment of an affinity tag to a second protein may be effected by cloning a nucleic acid encoding the affinity tag adjacent to a nucleic acid encoding a second protein in a nucleic acid vector.

As used herein, the term “glycosyltransferase,” refers to any enzyme/protein that has the ability to transfer a donor sugar to an acceptor moiety.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 70% identity, preferably 75%, 80%, 85%, 90%, 91%, 92%, 93%, 94%, 95%, 96%, 97%, 98%, 99%, or higher identity over a specified region (e.g., any one of SEQ ID NOs:1-12), when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or can be applied to, the compliment of a test sequence. The definition also includes sequences that have deletions and/or additions, as well as those that have substitutions. As described below, the preferred algorithms can account for gaps and the like. Preferably, identity exists over a region that is at least about 25, 50, 75, 100, 150, 200 amino acids or nucleotides in length, and oftentimes over a region that is 225, 250, 300, 350, 400, 450, 500 amino acids or nucleotides in length or over the full-length of am amino acid or nucleic acid sequences.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence can be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

“Conservatively modified variants” as used herein applies to amino acid sequences. One of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M) (see, e.g., Creighton, Proteins (1984)).

A “vector” is a composition of matter which comprises an isolated nucleic acid and which can be used to deliver the isolated nucleic acid to the interior of a cell. Numerous vectors are known in the art including, but not limited to, linear nucleic acids, nucleic acids associated with ionic or amphiphilic compounds, plasmids, and viruses (e.g., bacteriophages). Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like.

“Expression vector” refers to a vector comprising a recombinant nucleic acid comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant nucleic acid.

As used herein, the term “ST6GalNAcI” refers to N-acetylgalactosamine-α2,6-sialyltransferase I.

“ST6GalNAcI enzymatic or transferase activity” refers to the transfer of a sialic acid moiety from a nucleotide charged sialic acid donor moiety (e.g., a CMP-sialic acid or a sialic acid-polyethyleneglycol conjugate (SA-PEG)) in an α2→6 linkage to a N-acetylgalactosamine acceptor moiety.

As the term is used herein, a “truncated” form of a peptide refers to a peptide that is lacking one or more amino acid residues as compared to the full-length amino acid sequence of the peptide. For example, the peptide “NH2-Ala-Glu-Lys-Leu-COOH” is an N-terminally truncated form of the full-length peptide “NH2-Gly-Ala-Glu-Lys-Leu-COOH.” Truncation can be made from the N-terminal end, the C-terminal end, or from internal sequences within the polypeptide. The terms “truncated form” and “truncation mutant” are used interchangeably herein. By way of a non-limiting example, a truncated peptide is a ST6GalNAcI polypeptide comprising an active domain, a stem domain, a transmembrane domain, and a signal domain, wherein the signal domain is lacking a single N-terminal amino acid residue as compared to the full length ST6GalNAcI.

The term “saccharide” refers in general to any carbohydrate, a chemical entity with the most basic structure of (CH₂O)ₙ. Saccharides vary in complexity, and may also include nucleic acid, amino acid, or virtually any other chemical moiety existing in biological systems.

“Monosaccharide” refers to a single unit of carbohydrate of a defined identity.

“Oligosaccharide” refers to a molecule consisting of several units of carbohydrates of defined identity. Typically, saccharide sequences between 2-20 units may be referred to as oligosaccharides.

“Polysaccharide” refers to a molecule consisting of many units of carbohydrates of defined identity. However, any saccharide of two or more units may correctly be considered a polysaccharide.

As used herein, a saccharide “donor” is a moiety that can provide a saccharide to a glycosyltransferase so that the glycosyltransferase may transfer the saccharide to a saccharide acceptor. By way of a non-limiting example, a GalNAc donor may be UDP-GalNAc.

As used herein, a saccharide “acceptor” is a moiety that can accept a saccharide from a saccharide donor. A glycosyltransferase can covalently couple a saccharide to a saccharide acceptor. By way of a non-limiting example, G-CSF may be a GalNAc acceptor, and a GalNAc moiety may be covalently coupled to a GalNAc acceptor by way of a GalNAc-transferase.

The term “sialic acid” refers to any member of a family of nine-carbon carboxylated sugars. The most common member of the sialic acid family is N-acetyl-neuraminic acid (2-keto-5-acetamido-3,5-dideoxy-D-glycero-D-galactononulopyranos-1-onic acid (often abbreviated as Neu5Ac, NeuAc, or NANA). A second member of the family is N-glycolyl-neuraminic acid (Neu5Gc or NeuGc), in which the N-acetyl group of NeuAc is hydroxylated. A third sialic acid family member is 2-keto-3-deoxy-nonulosonic acid (KDN) (Nadano et al. (1986) J. Biol. Chem. 261: 11550-11557; Kanamori et al., J. Biol. Chem. 265: 21811-21819 (1990)). Also included are 9-substituted sialic acids such as a 9-O—C₁-C₆ acyl-Neu5Ac like 9-O-lactyl-Neu5Ac or 9-O-acetyl-Neu5Ac, 9-deoxy-9-fluoro-Neu5Ac and 9-azido-9-deoxy-Neu5Ac. For review of the sialic acid family, see, e.g., Varki, Glycobiology 2: 25-40 (1992); Sialic Acids Chemistry, Metabolism and Function, R. Schauer, Ed. (Springer-Verlag, New York (1992)). The synthesis and use of sialic acid compounds in a sialylation procedure is disclosed in international application WO 92/16640, published Oct. 1, 1992.

An “insoluble glycosyltransferase” refers to a glycosyltransferase that is expressed in bacterial inclusion bodies. Insoluble glycosyltransferases are typically solubilized or denatured using e.g., detergents or chaotropic agents or some combination. “Refolding” refers to a process of restoring the structure of a biologically active glycosyltransferase to a glycosyltransferase that has been solubilized or denatured. Thus, a refolding buffer, refers to a buffer that enhances or accelerates refolding of a glycosyltransferase.

A “redox couple” refers to mixtures of reduced and oxidized thiol reagents and include reduced and oxidized glutathione (GSH/GSSG), cysteine/cystine, cysteamine/cystamine, DTT/GSSG, and DTE/GSSG. (See, e.g., Clark, Cur. Op. Biotech. 12:202-207 (2001)).

The term “contacting” is used herein interchangeably with the following: combined with, added to, mixed with, passed over, incubated with, flowed over, etc.

The term “specific activity” as used herein refers to the catalytic activity of an enzyme, e.g., a recombinant glycosyltransferase fusion protein of the present invention, and may be expressed in activity units. As used herein, one activity unit catalyzes the formation of 1 μmol of product per minute at a given temperature (e.g., at 37° C.) and pH value (e.g., at pH 7.5). Thus, 10 units of an enzyme is a catalytic amount of that enzyme where 10 μmol of substrate are converted to 10 μmol of product in one minute at a temperature of, e.g., 37° C. and a pH value of, e.g., 7.5.

“N-linked” oligosaccharides are those oligosaccharides that are linked to a peptide backbone through asparagine, by way of an asparagine-N-acetylglucosamine linkage. N-linked oligosaccharides are also called “N-glycans.” All N-linked oligosaccharides have a common pentasaccharide core of Man₃GlcNAc₂. They differ in the presence of, and in the number of branches (also called antennae) of peripheral sugars such as N-acetylglucosamine, galactose, N-acetylgalactosamine, fucose and sialic acid. Optionally, this structure may also contain a core fucose molecule and/or a xylose molecule.

“O-linked” oligosaccharides are those oligosaccharides that are linked to a peptide backbone through threonine, serine, hydroxyproline, tyrosine, or other hydroxy-containing amino acids.

A “fusion protein” refers to a protein comprising amino acid sequences that are in addition to, in place of, less than, and/or different from the amino acid sequences encoding the original or native full-length protein or subsequences thereof.

A “chimeric” protein or “chimera” refers to a polypeptide sequence comprised of sequences from two or more different proteins. The two or more different proteins can be, for example, two homologues from two or more different species (e.g., human ST6GalNAcI and chimpanzee ST6GalNAcI), can be two different proteins from the same species (e.g., human ST6GalNAcI and human ST3 GalI), or can be two different proteins from two different species (e.g., porcine ST3GalI and human ST6GalNAcI). The sequence segments comprising the chimera can be contiguous or non-contiguous.

A “stem domain” with reference to glycosyltransferases refers to a protein domain, or a subsequence thereof, which in the native glycosyltransferases is located adjacent to the trans-membrane domain, and has been reported to function as a retention signal to maintain the glycosyltransferase in the Golgi apparatus and as a site of proteolytic cleavage. Stem domains generally start with the first hydrophilic amino acid following the hydrophobic transmembrane domain and end at the catalytic domain, or in some cases the first cysteine residue following the transmembrane domain. Exemplary stem domains include, but is not limited to, the stem domain of eukaryotic ST6GalNAcI, amino acid residues from about 30 to about 207 (see e.g., the murine enzyme), amino acids 35-278 for the human enzyme or amino acids 37-253 for the chicken enzyme; the stem domain of mammalian GalNAcI2, amino acid residues from about 71 to about 129 (see e.g., the rat enzyme).

A “catalytic domain” refers to a protein domain, or a subsequence thereof, that catalyzes an enzymatic reaction performed by the enzyme. For example, a catalytic domain of a sialyltransferase will include a subsequence of the sialyltransferase sufficient to transfer a sialic acid residue from a donor to an acceptor saccharide. A catalytic domain can include an entire enzyme, a subsequence thereof, or can include additional amino acid sequences that are not attached to the enzyme, or a subsequence thereof, as found in nature.

### DESCRIPTION

### I. Polypeptides

The modified ST6GalNAcI polypeptides can be modified from their native or wild-type counterparts in one or more of a number of ways, including truncation, modification of one or more cysteine residues, modification of one or more N-linked glycosylation sites, construction of a chimera of a ST6GalNAcI polypeptide with another polypeptide.

A modified ST6GalNAcI polypeptide can be a chimera with another polypeptide, for example, a ST6GalNAcI from another species, or another sialyltransferase (e.g., ST3GalI) from the same or another species. The portions of the chimera can be continuous or non-continuous. The ST6GalNAcI sequence segments and the sequence segment of the one or more other proteins can comprise the N-terminal region, the C-terminal region, or internal sequence regions. A chimeric protein can also have truncated regions, one or more modified cysteine residues or one or more modified N-linked glycosylation sites. Preferably the chimeras of the invention retain ST6GalNAcI transferase activity and have improved solubility (e.g., express in the soluble fraction of a prokaryotic host cell).

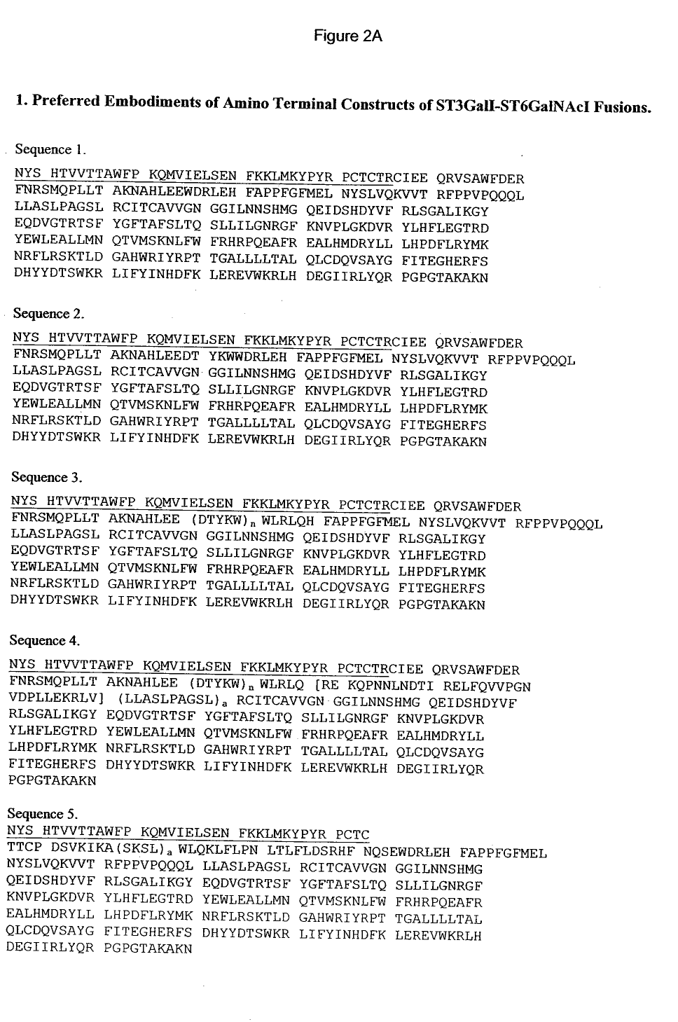

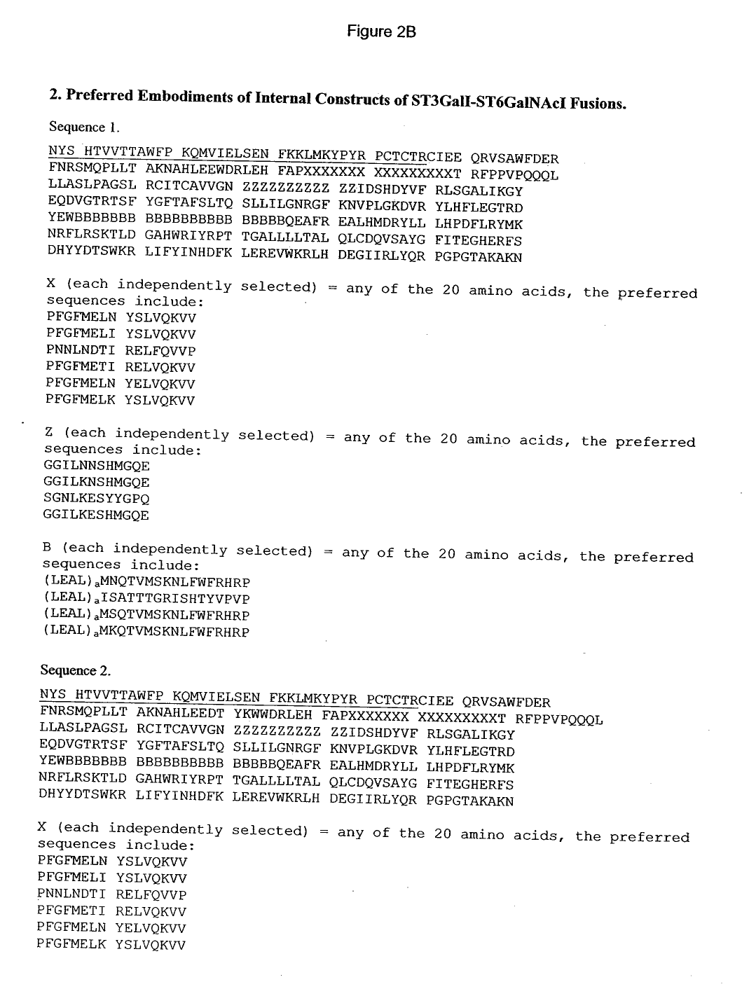

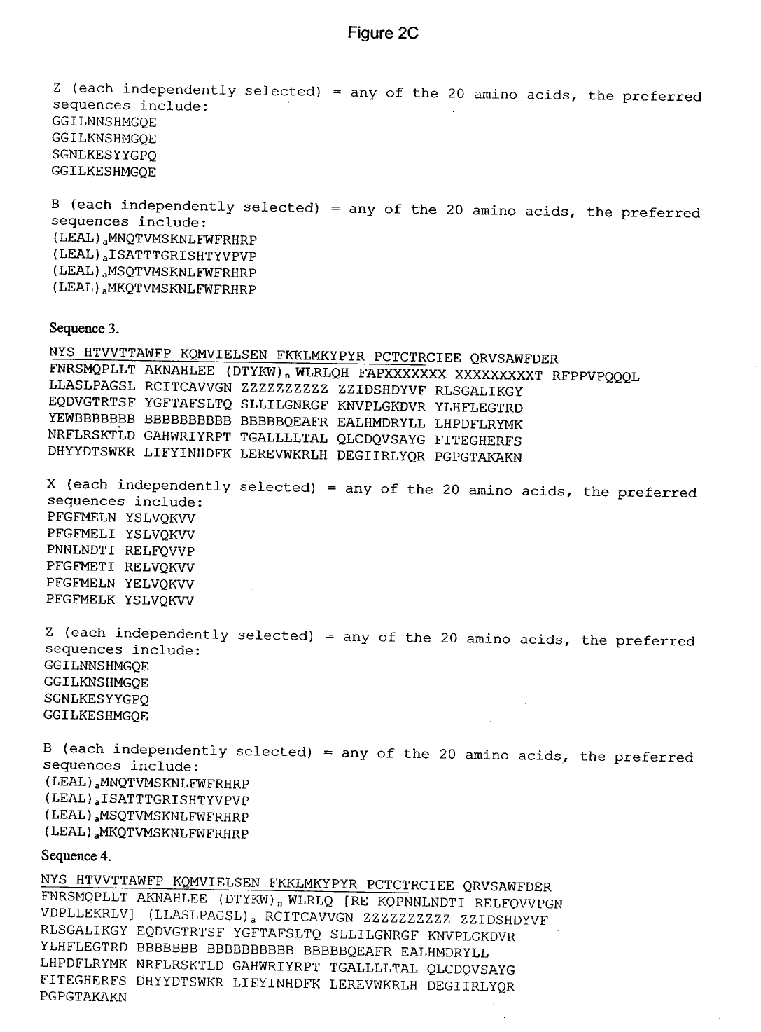

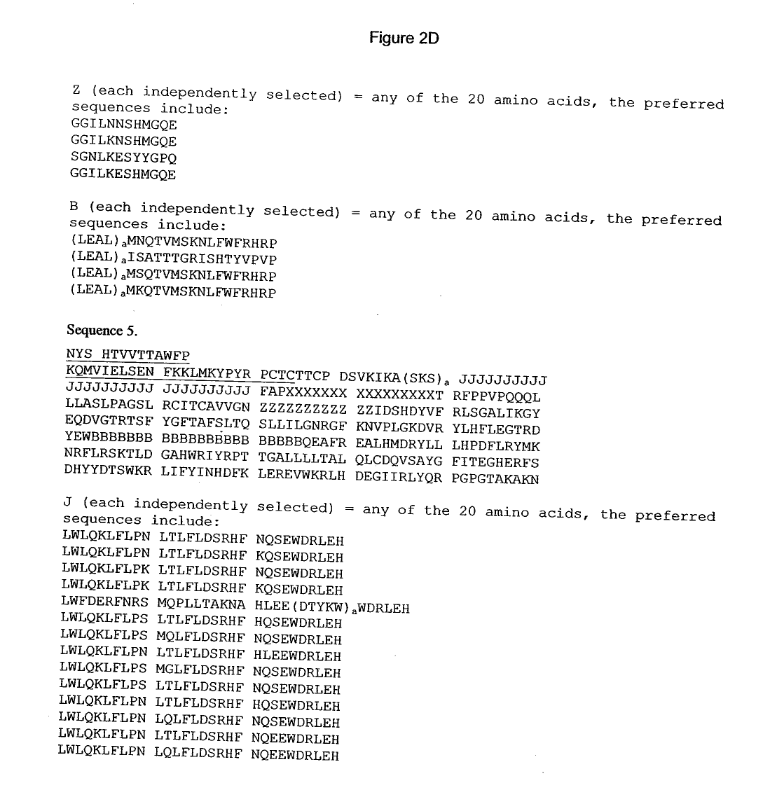

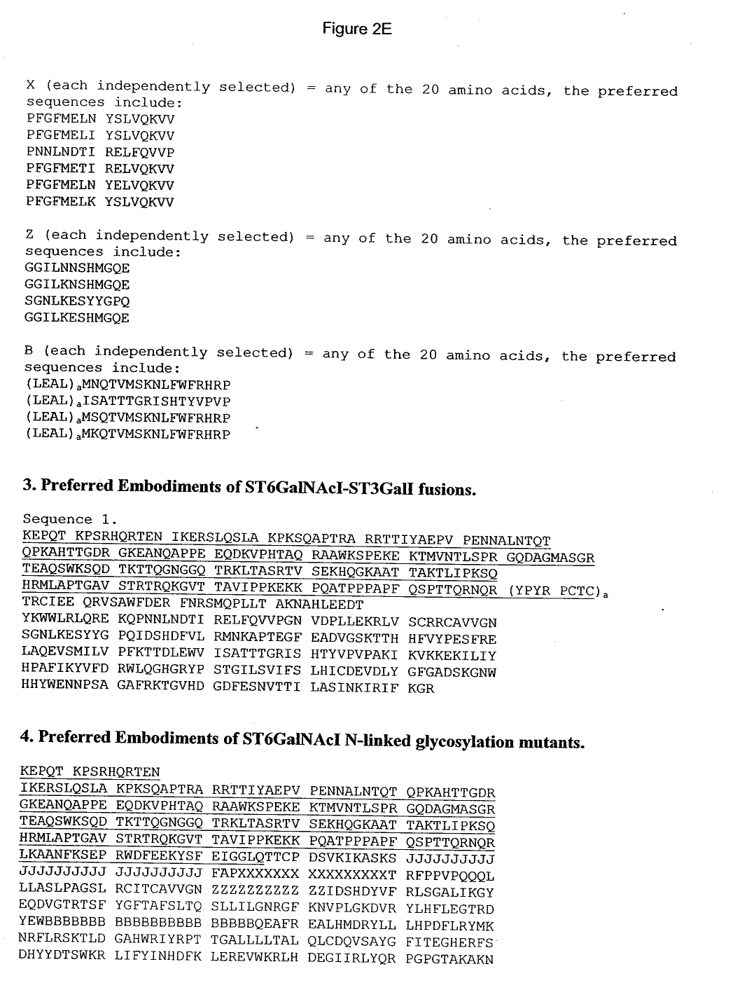

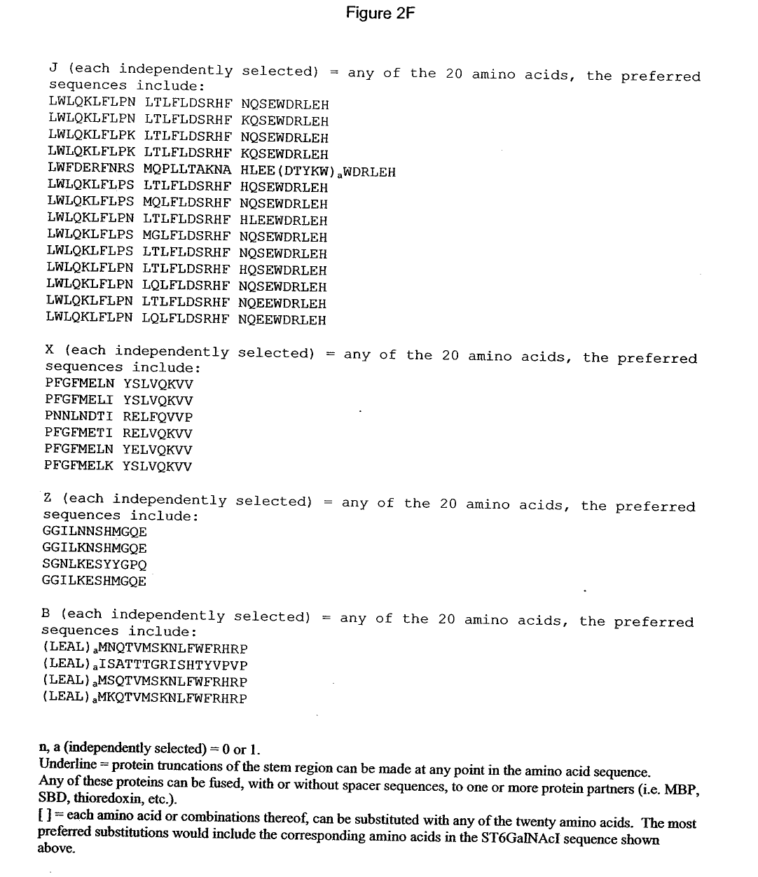

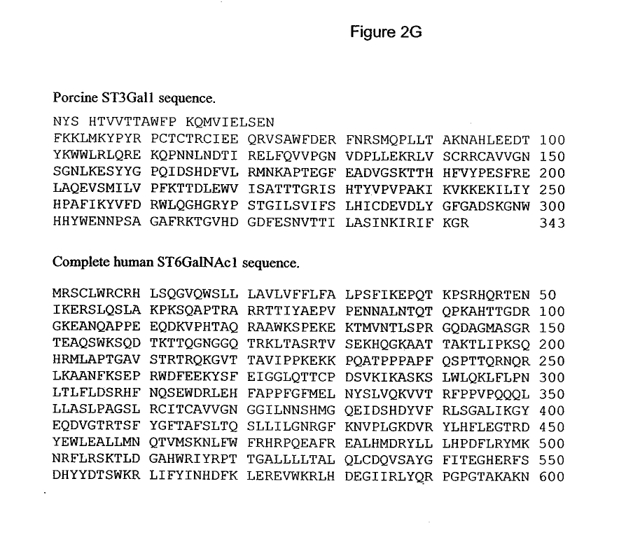

In a preferred embodiment, a chimera is comprised of amino acid sequence segments from a ST6GalNAcI polypeptide and a ST3 GalI polypeptide. Exemplified modified chimeric ST6GalNAc polypeptides, comprised of sequence segments from a human ST6GalNAcI and a porcine ST3 GalI, are shown in FIGS. 2A-2E. Modified ST6GalNAc polypeptides of the invention include amino acid sequences having ST6GalNAc transferase activity and having at least 90%, 91%, 92%, 93%, 94%, 95%, 96%, 97%, 98%, or 99% identity and 100% identity to the amino acid sequences exemplified in FIGS. 2A-2F.

A modified ST6GalNAcI polypeptide can be truncated in comparison to a native or wild-type counterpart. A truncated polypeptide of the present invention may be truncated in various ways, as would be known and understood by the skilled artisan, when armed with the disclosure set forth herein. Examples of truncated polypeptides of the present invention include, but are not limited to, a polypeptide lacking a single N-terminal residue, a polypeptide lacking a single C-terminal residue, a polypeptide lacking both an single N-terminal residue and a single C-terminal residue, a polypeptide lacking a contiguous sequence of residues from the N-terminus, a polypeptide lacking a contiguous sequence of residues from the C-terminus, and any such combinations thereof.

As would be understood by the skilled artisan, a full-length human ST6GalNAcI polypeptide may contain one or more identifyable polypeptide domains in addition to the “active domain,” the domain primarily responsible for the catalytic activity, of ST6GalNAcI. This is because it is known in that art that a full-length ST6GalNAcI polypeptide contains a signal domain, a transmembrane domain, and a stem domain, in addition to an active domain. Accordingly, a full-length ST6GalNAcI may have a signal domain at the amino-terminus of the polypeptide, followed by a transmembrane domain immediately adjacent to the signal domain, followed by a stem domain that is immediately adjacent to the transmembrane domain, followed by an active domain that extends to the carboxy-terminus of the polypeptide and is located immediately adjacent to the stem domain.

A modified ST6GalNAcI polypeptide of the invention can also be a truncated polypeptide lacking all or a portion of a signal domain (e.g, from ST6GalNAcI or another protein fused to ST6GalNAcI, including ST3GalI). In another embodiment, a modified ST6GalNAcI polypeptide of the invention is a truncated polypeptide lacking the a signal domain and all or a portion of the a transmembrane domain (e.g, from ST6GalNAcI or another protein fused to ST6GalNAcI, including ST3 GalI). In yet another embodiment, a modified ST6GalNAcI polypeptide of the invention is a truncated polypeptide lacking a signal domain, a transmembrane domain and all or a portion a stem domain (e.g, from ST6GalNAcI or another protein fused to ST6GalNAcI, including ST3 GalI). Exemplified truncated ST6GalNAcI polypeptides are described, for example in International Application Publication No. WO 2005/121332, the disclosures of each of which are hereby incorporated herein by reference in their entirety for all purposes.

A modified ST6GalNAcI polypeptide can also have one or more cysteine residues modified. One or more cysteine residues can be deleted, changed or added in comparison to a native or wild-type sequence. For one or more cysteine residues at positions 280, 362, 365 and 533 in a human ST6GalNAcI polypeptide sequence, or corresponding positions in aligned ST6GalNAcI polypeptide sequence, can be changed to a threonine or serine residue.

A modified ST6GalNAcI polypeptide can also have one or more N-linked glycosylation sites modified. One or more new or additional N-linked glycosylation sites can be introduced in comparison to a native sequence. Also, one or more N-linked glycosylation sites can be deleted or changed in comparison to a native sequence. An exemplified ST6GalNAcI polypeptide with one or more N-linked glycosylation sites modified is shown in FIG. 2F.

The present invention also provides for analogs of polypeptides which comprise a modified ST6GalNAcI polypeptide as disclosed herein. Analogs can differ from naturally occurring proteins or peptides by conservative amino acid sequence differences or by modifications which do not affect sequence, or by both.

For example, conservative amino acid changes may be made, which although they alter the primary sequence of the protein or peptide, do not normally alter its function. Conservative amino acid substitutions typically include substitutions within the following groups:
glycine, alanine;
valine, isoleucine, leucine;
aspartic acid, glutamic acid;
asparagine, glutamine;
serine, threonine;
lysine, arginine;
phenylalanine, tyrosine.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The peptides of the invention are not limited to products of any of the specific exemplary processes listed herein.

Fragments of a modified ST6GalNAcI polypeptide of the invention are included in the present invention, provided the fragment possesses the biological activity of the full-length polypeptide. That is, a modified ST6GalNAcI polypeptide of the present invention can catalyze the same glycosyltransfer reaction as the full-length ST6GalNAcI. By way of a non-limiting example, a modified human ST6GalNAcI polypeptide of the invention has the ability to transfer a sialic acid moiety from a CMP-sialic acid donor to a bovine submaxillary mucin acceptor, wherein such a transfer results in the covalent coupling of a sialic acid moiety to a GalNAc residue on the bovine submaxillary mucin acceptor. Therefore, a smaller than full-length and modified ST6GalNAcI is included in the present invention provided that the modified ST6GalNAcI has ST6GalNAcI biological activity.

In another aspect of the present invention, compositions comprising a modified ST6GalNAcI polypeptide as described herein may include purified and highly purified modified ST6GalNAcI polypeptides. Alternatively, compositions comprising modified ST6GalNAcI polypeptides can include cell lysates prepared from the prokaryotic host cells used to express the particular modified ST6GalNAcI polypeptides. Further, modified ST6GalNAcI polypeptides of the present invention may be expressed in one of any number of prokaryotic cells suitable for expression of polypeptides, such cells being well-known to one of skill in the art, as described in detail elsewhere herein.

It will be appreciated that all of the above descriptions of a modified ST6GalNAcI polypeptide apply equally to modified ST6GalNAcI polypeptides of the invention from any source, including, but not limited to mammalian ST6GalNAcI, human ST6GalNAcI, chimpanzee ST6GalNAcI, orangutan ST6GalNAcI, pig ST6GalNAcI, cow ST6GalNAcI, dog ST6GalNAcI, rat ST6GalNAcI, mouse ST6GalNAcI, and chicken ST6GalNAcI. Native or wild-type ST6GalNAcI and ST3 GalI polypeptide sequences from numerous animal species, including human, chimpanzee, cow, pig, dog, rat, mouse and chicken are known in the art. Exemplified ST6GalNAcI native polypeptide sequences include GenBank accession numbers CAA72179 (human), Q9NSC7 (human), CA129584 (orangutan), NP_{—}035501 (mouse), Q9QZ39 (mouse), NP_{—}990571 (chicken), XP_{—}540453 (dog), XP_{—}876077 (cow), CAG25684 (rat), and CAG38615 (chimpanzee). Exemplified ST3GalI native polypeptide sequences include GenBank accession numbers A45073 (pig), AAA31125 (pig), NP_{—}033203 (mouse), NP_{—}990548 (chicken), NP_{—}001009037 (chimpanzee), and NP_{—}003024 (human).

Substantially pure protein isolated and obtained as described herein may be purified by following known procedures for protein purification, wherein an immunological, enzymatic or other assay is used to monitor purification at each stage in the procedure. Protein purification methods are well known in the art, and are described, for example in Deutscher et al. (ed., 1990, Guide to Protein Purification, Harcourt Brace Jovanovich, San Diego).

In a preferred embodiment, the modified ST6GalNAc I polypeptides of the invention are fused to a purification tag, e.g., a maltose binding domain (MBD) tag or a starch binding domain (SBD) tag. Such modified ST6GalNAc I fusion proteins can be purified by passage through a column that specifically binds to the purification tag, e.g., MBD or SBD proteins can be purified on a cyclodextrin column. In a further embodiment, a modified ST6GalNAc I fusion proteins comprising a purification tag, such as, e.g., an MBD or SBD tag, are immobilized on a column that specifically binds to the purification tag and substrates, e.g., a sialic acid donor or PEGylated-sialic acid donor and a glycoprotein or glycopeptide comprising an O-linked glycylation site are passed through the column under conditions that facilitate transfer of sialic acid from a donor, e.g., CMP-sialic acid or CMP-PEGylated-sialic acid, to a glycoprotein or glycopeptide acceptor, and thus production of a sialylated glycoprotein or sialylated glycopeptide.

The polypeptides of the invention can be made by constructing an appropriate nucleic acid sequence, below. Also, the modified polypeptides of the invention can be synthetically produced according to methods well known in the art. See, for example, Applications of Chimeric Genes and Hybrid Proteins: Gene Expression and Protein Purification, Methods in Enzymology Vol. 326, Thomer, et al., Academic Press, 2000; and Protein Synthesis: Method and Protocols, Martin, ed., Humana Press, 1998.

II. Nucleic acids

The present invention features nucleic acids encoding the modified ST6GalNAcI polypeptides described herein. That is, the present invention features a nucleic acid encoding a modified ST6GalNAcI polypeptide, provided the polypeptide expressed by the nucleic acid retains the biological activity of a full-length or native or wild-type ST6GalNAcI protein (i.e., the ability to transfer a sialic acid moiety from a sialic acid donor to an acceptor molecule). The nucleic acids encode modified ST6GalNAcI polypeptides having one or more of the following modifications: one or more truncated regions, one or more modified cysteine residues, one or more modified N-linked glycosylation sites, or the construction of a chimeric ST6GalNAcI protein with another protein (e.g., a ST3GalI protein).

In one embodiment, the nucleic acids encode a chimeric polypeptide comprised of sequences of a ST6GalNAcI polypeptide and a ST3GalI polypeptide, as described above. In preferred embodiments, the nucleic acid sequences encode a chimeric polypeptide having at least 90%, 91%, 92%, 93%, 94%, 95%, 96%, 97%, 98%, or 99% identity and 100% identity to the amino acid sequences exemplified in FIGS. 2A-2F.

In another embodiment, the nucleic acids encode a modified ST6GalNAcI polypeptide having one or more modified cysteine residues, as described above. In another embodiment, the nucleic acids encode a modified ST6GalNAcI polypeptide having one or more modified N-linked glycosylation sites, as described above. In a preferred embodiment, the nucleic acids encode a polypeptide having N-linked glycosylation sites modified as shown in FIG. 2F.

The nucleic acids of the invention can also encode a modified ST6GalNAcI polypeptide that is truncated in comparison to a native or wild-type sequence, as described above, wherein the modified ST6GalNAcI polypeptide is lacking all or a portion of a signal domain (from a native ST6GalNAcI or another protein fused to ST6GalNAcI, including ST3 GalI). In another embodiment, an nucleic acid of the invention encodes a truncated polypeptide, wherein the modified ST6GalNAcI polypeptide is lacking a signal domain and all or a portion of a transmembrane domain (from a native ST6GalNAcI or another protein fused to ST6GalNAcI, including ST3GalI). In yet another embodiment, a nucleic acid of the invention may encode a truncated polypeptide, wherein the modified ST6GalNAcI polypeptide is lacking a signal domain, a transmembrane domain and all or a portion the a stem domain (from a native ST6GalNAcI or another protein fused to ST6GalNAcI, including ST3GalI).

The methods and compositions of the invention should not be construed to be limited solely to a nucleic acid comprising a modified ST6GalNAcI polypeptide or polynucleotide as disclosed herein, but rather, should be construed to encompass any nucleic acid encoding any modified ST6GalNAcI polypeptide or polynucleotide, prepared in accordance with the disclosure herein, either known or unknown, which is capable of catalyzing transfer of a sialic acid to a sialic acid acceptor.

It will be appreciated that all of the above descriptions of a modified ST6GalNAcI polynucleotide apply equally to modified ST6GalNAcI nucleotides of the invention from any source, including, but not limited to mammalian ST6GalNAcI, human ST6GalNAcI, chimpanzee ST6GalNAcI, pig ST6GalNAcI, cow ST6GalNAcI, dog ST6GalNAcI, rat ST6GalNAcI, mouse ST6GalNAcI, and chicken ST6GalNAcI. Native or wild-type ST6GalNAcI and ST3GalI nucleic acid sequences from numerous animal species, including human, chimpanzee, cow, pig, dog, rat, mouse and chicken are well known in the art. Exemplified ST6GalNAcI native polynucleotide sequences include GenBank accession numbers NM_{—}018414 (human), NM_{—}011371 (mouse), NM_{—}205240 (chicken), AJ748740 (chimpanzee), CR925921 (orangutan), L29554 (rat), XM_{—}870984 (cow), and XM_{—}540453 (dog). Exemplified ST3GalI native polynucleotide sequences include GenBank accession numbers NM_{—}003033 human), NM_{—}173344 (human), NM_{—}001009037 (chimpanzee), NM_{—}001004047 (pig), NM_{—}009177 (mouse), and NM_{—}205217 (chicken).

Techniques for introducing changes in nucleotide sequences that are designed to alter the functional properties of the encoded proteins or polypeptides are well known in the art. Such modifications include the deletion, insertion, or substitution of bases, and thus, changes in the amino acid sequence. As is known to one of skill in the art, nucleic acid insertions and/or deletions may be designed into the gene for numerous reasons, including, but not limited to modification of nucleic acid stability, modification of nucleic acid expression levels, modification of expressed polypeptide stability or half-life, modification of expressed polypeptide activity, modification of expressed polypeptide properties and characteristics, and changes in glycosylation pattern. All such modifications to the nucleotide sequences encoding such proteins are encompassed by the present invention.

Alternatively, nucleic acid sequences encoding modified ST6GalNAcI polypeptides of the invention can be conveniently made as completely synthetic sequences. Techniques for constructing synthetic nucleic acid sequences encoding a protein or synthetic gene sequences are well known in the art. Synthetic gene sequences can be commercially purchased through any of a number of service companies, including DNA 2.0 (Menlo Park, Calif.), Geneart (Toronto, Ontario, Canada), CODA Genomics (Irvine, Calif.), and GenScript, Corporation (Piscataway, N.J.).

It is not intended that methods and compositions of the present invention be limited by the nature of the nucleic acid employed. The target nucleic acid encompassed by methods and compositions of the invention may be native or synthesized nucleic acid. The nucleic acid may be DNA or RNA and may exist in a double-stranded, single-stranded or partially double-stranded form. Furthermore, the nucleic acid may be found as part of a virus or other macromolecule. See, e.g., Fasbender et al., 1996, J. Biol. Chem. 272:6479-89.

### III. Vectors and Expression Systems

In other related aspects, the invention includes a nucleic acid encoding a modified ST6GalNAcI polypeptide operably linked to a nucleic acid comprising a promoter/regulatory sequence such that the nucleic acid is preferably capable of directing expression of the polypeptide encoded by the nucleic acid. Thus, the invention encompasses expression vectors and methods for the introduction of exogenous DNA into cells with concomitant expression of the exogenous DNA in those cells, as described, for example, in Sambrook et al. (Third Edition, 2001, Molecular Cloning: A Laboratory Manual, Cold Spring Harbor Laboratory, New York), and in Ausubel et al. (1987-2005, Current Protocols in Molecular Biology, John Wiley & Sons, New York).

Expression of a modified ST6GalNAcI polypeptide in a prokaryotic host cell can be accomplished by generating a plasmid, viral, or other type of vector comprising a nucleic acid encoding the appropriate nucleic acid, wherein the nucleic acid is operably linked to a promoter/regulatory sequence which serves to drive expression of the encoded polypeptide, with or without tag, in cells in which the vector is introduced. In addition, promoters which are well known in the art which are induced in response to inducing agents such as metals, saccharides and saccharide analogs, and the like, are also contemplated in the invention. Thus, it will be appreciated that the invention includes the use of any promoter/regulatory sequence, which is either known or unknown, and which is capable of driving expression of the modified ST6GalNAcI polypeptide operably linked thereto.

In an expression system useful in the present invention, a nucleic acid encoding a modified ST6GalNAcI polypeptide can be further fused to one or more additional nucleic acids encoding a functional polypeptide. By way of a non-limiting example, an affinity tag coding sequence can be inserted into a nucleic acid vector adjacent to, upstream from, or downstream from a modified ST6GalNAcI polypeptide coding sequence. As will be understood by one of skill in the art, an affinity tag will typically be inserted into a multiple cloning site in frame with the modified ST6GalNAcI polypeptide. One of skill in the art will also understand that an affinity tag coding sequence can be used to produce a recombinant fusion protein by concomitantly expressing the affinity tag and modified ST6GalNAcI polypeptide. The expressed fusion protein can then be isolated, purified, or identified by means of the affinity tag.

Affinity tags useful in the present invention include, but are not limited to, a maltose binding protein (MBP), a histidine tag, a Factor IX tag, a glutathione-S-transferase (GST) tag, a FLAG-tag, and a starch binding domain (SBD) tag. Other tags are well known in the art, and the use of such tags in the present invention would be readily understood by the skilled artisan.

As would be understood by one of skill in the art, a vector comprising a modified ST6GalNAcI polypeptide of the present invention may be used to express the modified polypeptide as either a non-fusion or as a fusion protein. Selection of any particular plasmid vector or other DNA vector is not a limiting factor in this invention and a wide plethora of vectors are well-known in the art. Further, it is well within the skill of the artisan to choose particular promoter/regulatory sequences and operably link those promoter/regulatory sequences to a DNA sequence encoding either a modified ST6GalNAcI polypeptide. Such technology is well known in the art and is described, for example, in Sambrook et al. supra and in Ausubel et al., supra. By way of a non-limiting example, vectors useful in expressing the modified ST6GalNAc polypeptides of the present invention include those based on the pcWori+ vector (Muchmore et al., 1987, Meth. Enzymol. 177:44-73), and the pCWIN2-MBP vector, described, for example, in U.S. Provisional Application No. 60/535,263 (published as International Application Publication No. WO 2005/067601), the disclosures of each of which are hereby incorporated herein by reference in their entirety for all purposes.

The invention thus includes a vector comprising a nucleic acid encoding a modified ST6GalNAcI polypeptide. The incorporation of a nucleic acid into a vector and the choice of vectors is well-known in the art as described in, for example, Sambrook et al. supra, and in Ausubel et al., supra.

In an aspect of the invention, an isolated nucleic acid encoding a modified ST6GalNAcI polypeptide is integrated into the genome of a host cell in conjunction with a nucleic acid encoding a modified ST6GalNAcI polypeptide. In another aspect of the invention, a cell is transiently transfected with an isolated nucleic acid encoding a modified ST6GalNAcI polypeptide. In another aspect of the invention, a cell is stably transfected with a nucleic acid encoding a modified ST6GalNAcI polypeptide.

For the purpose of inserting an isolated nucleic acid into a cell, one of skill in the art would also understand that the methods available and the methods required to introduce an isolated nucleic acid of the invention into a host cell vary and depend upon the choice of host cell. Suitable methods of introducing an isolated nucleic acid into a host cell are well-known in the art. Other suitable methods for transforming or transfecting host cells may include, but are not limited to, those found in Sambrook, et al. supra, and other such laboratory manuals.

A nucleic acid encoding a modified ST6GalNAcI polypeptide may be purified by any suitable means, as are well known in the art. For example, the nucleic acids can be purified by reverse phase or ion exchange HPLC, size exclusion chromatography or gel electrophoresis. Of course, the skilled artisan will recognize that the method of purification will depend in part on the size of the DNA to be purified.

The present invention also features a recombinant bacterial or prokaryotic host cell comprising, inter alia, a nucleic acid vector as described elsewhere herein. In one aspect, the recombinant cell is transformed with a vector of the present invention. The transformed vector need not be integrated into the cell genome nor does it need to be expressed in the cell. However, the transformed vector will be capable of being expressed in the cell. Exemplified prokaryotic host cells include bacillales (Bacillus) and proteobacteria, including alphaproteobacteria (e.g., Caulobacterales), betaproteobacteria (e.g., Burkholderialies, including Ralstonia), and gammaproteobacteria (e.g., Pseudomonadales (Pseudomonas fluorescens group) and Enterobacteriales (Escherichia coli). In one aspect of the invention, a Bacillus subtilis cell is used for transformation of a vector of the present invention and expression of protein therefrom. In another aspect of the invention, Escherichia coli is used for transformation of a vector of the present invention and expression of protein therefrom. In another aspect of the invention, a K-12 strain of E. coli is useful for expression of protein from a vector of the present invention. Strains of E. coli useful in the present invention include, but are not limited to, JM83, JM101, JM103, JM109, W3110, chi1776, BNN93 and JA221. Commercially available E. coli host strains of use in expressing modified ST6GalNAcI polypeptides include, for example, Tuner™, Rosetta™, Origami, OrigamiB, Rosetta-gami, purchasable from Novagen/EMD Biosciences, San Diego, Calif. The use of such strains is described, for example, in U.S. Patent Publication No. 2006/234345 (also published as WO 2006/102652); International Application Publication No. WO 2005/121332; and International Application No. PCT/US06/34844, Attorney Docket No. 019957-021310PC, entitled “Expression of Soluble Therapeutic Proteins,” filed Sep. 1, 2006, the disclosures of each of which are herein incorporated by reference in their entirety for all purposes.

It will be understood that a host cell useful in the present invention will be capable of growth and culture on a small scale, medium scale, or a large scale. For example, a host cell of the invention is useful for testing the expression of a protein from a vector of the invention equally as much as it is useful for large scale production of a reagent or therapeutic protein product. Techniques useful in culturing host cells and expressing protein from a vector contained therein are well known in the art and will therefore not be listed herein. See, for example, Prokaryotic Gene Expression, Baumberg, ed., 1999, Oxford University Press.

A host cell useful in methods of the present invention, as described above, can be prepared according to various methods, as would be understood by the skilled artisan when armend with the disclosure set forth herein. In one aspect, a host cell of the present invention may be transformed with a vector of the present invention to produce a transformed host cell of the invention. Transformation, as known to the skilled artisan, includes the process of inserting a nucleic acid vector into a host cell, such that the host cell containing the nucleic acid vector remains viable. Such transformation of nucleic acid into a bacterial cell is useful for purposes including, but not limited to, creation of a stably-transformed host cell, making a biological deposit, propagating the vector-containing host cell, propagating the vector-containing host cell for the production and isolation of additional vector, expression of target protein encoded by vector, and the like.

Methods of transforming a cell with a vector are numerous and well-known in the art, and will therefore not be listed here. By way of a non-limiting example, a competent bacterial cell of the invention may be transformed by a vector of the invention using electroporation. Methods of making bacterial cells “competent” are well-known in the art, and typically involve preparation of the bacterial cells so that the cells take up exogenous DNA. Similarly, methods of electroporation are known in the art, and detailed descriptions of such methods may be found, for example, in Sambrook et al. (1989, supra). The transformation of a competent cell with vector DNA may be also accomplished using chemical-based methods. One example of a well-known chemical-based method of bacterial transformation is described by Inoue, et al. (1990, Gene 96:23-28). Other methods of transformation will be known to the skilled artisan.

A transformed host cell of the present invention can be used to express a modified ST6GalNAcI polypeptide of the present invention. In an embodiment of the invention, a transformed host cell contains a vector of the invention, which contains therein a nucleic acid sequence encoding an modified polypeptide of the invention. The modified polypeptide is expressed using any expression method known in the art (for example, IPTG). The expressed modified polypeptide may be contained within the host cell, or it may be secreted from the host cell into the growth medium.

Methods for isolating an expressed polypeptide are well-known in the art, and the skilled artisan will know how to determine the best method for isolation of an expressed polypeptide based on the characteristics of any given host cell expression system. By way of a non-limiting example, an expressed polypeptide that is secreted from a host cell may be isolated from the growth medium. Isolation of a polypeptide from a growth medium may include removal of bacterial cells and cellular debris. By way of another non-limiting example, an expressed polypeptide that is contained within a host cell may be isolated from the host cell. Isolation of such an “intracellular” expressed polypeptide may include disruption of the host cell and removal of cellular debris from the resultant mixture. These methods are not intended to be exclusive representations of the present invention, but rather, are merely for the purposes of illustration of various applications of the present invention.

Purification of a modified polypeptide expressed in accordance with the present invention may be effected by any means known in the art. The skilled artisan will know how to determine the best method for the purification of a polypeptide expressed in accordance with the present invention. A purification method will be chosen by the skilled artisan based on factors such as, but not limited to, the expression host, the contents of the crude extract of the polypeptide, the size of the polypeptide, the properties of the polypeptide, the desired end product of the polypeptide purification process, and the subsequent use of the end product of the polypeptide purification process. See, for example, Scopes, Protein Purification: Principles and Practice, Springer-Verlag (January 1994).

In an embodiment of the invention, isolation or purification of a modified polypeptide expressed in accordance with the present invention may not be desired. In an aspect of the present invention, an expressed polypeptide may be stored or transported inside the bacterial host cell in which the polypeptide was expressed. In another aspect of the invention, an expressed polypeptide may be used in a crude lysate form, which is produced by lysis of a host cell in which the polypeptide was expressed. In yet another embodiment of the invention, an expressed polypeptide may be partially isolated or partially purified according to any of the methods set forth or described herein. The skilled artisan will know when it is not desirable to isolate or purify a polypeptide of the invention, and will be familiar with the techniques available for the use and preparation of such polypeptides.

### IV. Methods

The present invention features a method of expressing a modified ST6GalNAcI polypeptide, as described above, according to methods for expressing proteins in prokaryotic host cells well known in the art. See, Thomer and Baumberg, supra. More preferably, polypeptides which can be expressed according to the methods of the present invention include, but are not limited to, a modified ST6GalNAcI polypeptide having one or more of the following modifications: a constructed chimera with one or more other proteins (e.g., an ST3GalI polypeptide), one or more truncated regions, one or more modified cysteine residues or one or more modified N-linked glycosylation sites. In a preferred embodiment, a polypeptide which can be expressed according to the methods of the present invention is a polypeptide comprising any one of the polypeptide sequences set forth in FIGS. 2A-2F.

In one embodiment, the present invention features a method of expressing a modified ST6GalNAcI polypeptide encoded by an isolated nucleic acid of the invention, as described elsewhere herein, wherein the expressed modified ST6GalNAcI polypeptide has the property of catalyzing the transfer of a sialic acid moiety to an acceptor moiety. In one aspect of the invention, a method of expressing a modified ST6GalNAcI polypeptide includes the steps of cloning an isolated nucleic acid of the invention into an expression vector, inserting the expression vector construct into a host cell, and expressing a modified ST6GalNAcI polypeptide therefrom.

Methods of expression of polypeptides, as well as construction of expression systems and recombinant host cells for expression of polypeptides, are discussed in extensive detail elsewhere herein. Methods of expression of a modified polypeptide of the present invention will be understood to include, but not to be limited to, all such methods as described herein. In some expression systems, the modified ST6GalNAcI polypeptides of the invention are expressed as insoluble proteins, e.g., in an inclusion protein in a bacterial host cell. Methods of refolding insoluble glycosyltransferases, including ST6GalNAcI polypeptides, are disclosed in U.S. patent application Ser. No. 10/587,769 (published as WO 2005/089102); U.S. Patent Publication No. 2006/234345 (also published as WO 2006/102652); International Patent Publication No. WO 2005/121332; and International Patent No. PCT/US06/34844, Attorney Docket No. 019957-021310PC, entitled “Expression of Soluble Therapeutic Proteins,” filed Sep. 1, 2006, the disclosures of each of which are herein incorporated by reference in their entirety for all purposes.

The present invention also features a method of catalyzing a glycosyltransferase reaction between a glycosyl donor and a glycosyl acceptor. In one embodiment, the invention features a method catalyzing the transfer of a sialic acid moiety to an acceptor moiety, wherein the sialyltransfer reaction is carried out by incubating a modified ST6GalNAcI polypeptide of the invention with a sialic acid donor moiety and an acceptor moiety. In one aspect, a modified ST6GalNAcI polypeptide of the invention mediates the covalent linkage of a sialic acid moiety to an acceptor moiety, thereby catalyzing the transfer of a sialic acid moiety to an acceptor moiety.

In an embodiment of the invention, a modified ST6GalNAcI polypeptide useful in a glycosyltransfer reaction is a modified human, chimpanzee, mouse, rat, cow, pig, dog or chicken ST6GalNAcI polypeptide. In another embodiment, a modified ST6GalNAcI polypeptide useful in a glycosyltransfer reaction is a chimera with another protein, for example a ST3 GalI protein. In a preferred embodiment, a modified ST6GalNAcI polypeptide useful in a glycosyltransfer reaction is a polypeptide comprising any one of the polypeptide sequences set forth in FIGS. 2A-2F.

By way of a non-limiting example, a method of catalyzing the transfer of a sialic acid moiety to an acceptor moiety includes the steps of incubating a modified human ST6GalNAcI polypeptide with a cytidinemonophosphate-sialic acid (CMP-NAN) sialic acid donor and an asialo bovine submaxillary mucin acceptor moiety, wherein the modified human ST6GalNAcI polypeptide mediates the transfer of a sialic acid moiety from CMP-NAN to the bovine submaxillary mucin acceptor.

Therefore, in one embodiment, the present invention also features a polypeptide acceptor moiety. In one embodiment of the invention, a polypeptide acceptor moiety is a human growth hormone. In another embodiment, a polypeptide acceptor moiety is an erythropoietin. In yet another embodiment, a polypeptide acceptor moiety is an interferon-alpha. In another embodiment, a polypeptide acceptor moiety is an interferon-beta. In another embodiment of the invention, a polypeptide acceptor moiety is an interferon-gamma. In still another embodiment of the invention, a polypeptide acceptor moiety is a lysosomal hydrolase. In another embodiment, a polypeptide acceptor moiety is a blood factor polypeptide. In still another embodiment, a polypeptide acceptor moiety is an anti-tumor necrosis factor-alpha. In another embodiment of the invention, a polypeptide acceptor moiety is follicle stimulating hormone. In yet another embodiment of the invention, a polypeptide acceptor moiety is a glucagon-like peptide.

In one embodiment, the present invention also features a method of transferring a sialic acid-polyethyleneglycol conjugate (SA-PEG) to an acceptor molecule. In one aspect, an acceptor molecule is a polypeptide. In another aspect, an acceptor molecule is a glycopeptide. Compositions and methods useful for designing, producing and transferring a SA-PEG conjugate to an acceptor molecule are discussed at length in International (PCT) Patent Application No. WO 03/031464 (PCT/US02/32263) and U.S. Patent Application No. 2004/0063911, each of which is incorporated herein by reference in its entirety.

Methods of assaying for glycosyltransferase activity are well-known in the art. Various assays for detecting glycosyltransferases which can be used in accordance with the invention have been published. The following are illustrative, but should not be considered limiting, of those assays useful for detecting glycosyltransferase activity. Furukawa et al (1985, Biochem. J., 227:573-582) describe a borate-impregnated paper electrophoresis assay and a fluorescence assay. Roth et al (1983, Exp'l Cell Research 143:217-225) describe application of the borate assay to glucuronyl transferases, previously assayed calorimetrically. Benau et al (1990, J. Histochem. Cytochem., 38:23-30) describe a histochemical assay based on the reduction, by NADH, of diazonium salts. See also U.S. Pat. No. 6,284,493 of Roth, incorporated herein by reference.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A chimeric polypeptide comprising a first portion from a Gal-β-1,3GalNAc-α-2,3-sialyltransferase (ST3GalI) polypeptide and a second portion from a GalNAc-α-2,6-sialyltransferase I (ST6GalNAcI) polypeptide, wherein the polypeptide has ST6GalNAcI transferase activity.

2. The polypeptide of claim 1, wherein the first portion and the second portion are contiguous.

3. The polypeptide of claim 1, wherein the first portion and the second portion are not contiguous.

4. The polypeptide of claim 1, wherein the first portion from a ST3GalI polypeptide comprises an N-terminal sequence segment of the chimeric polypeptide and the second portion from a ST6GalNAcI polypeptide comprises a C-terminal sequence segment of the chimeric polypeptide.

5. The polypeptide of claim 4, wherein the N-terminal sequence from the ST3 GalI polypeptide portion lacks one or more amino acid residues from the signal peptide.

6. The polypeptide of claim 5, wherein the N-terminal sequence from the ST3GalI polypeptide portion lacks one or more amino acid residues from the transmembrane domain.

7. The polypeptide of claim 6, wherein the N-terminal sequence from the ST3GalI polypeptide portion lacks one or more amino acid residues from the stem domain.

8. The polypeptide of claim 1, wherein the ST3GalI polypeptide portion comprises one or more sequence segments inserted internally within the ST6GalNAcI polypeptide portion.

9. The polypeptide of claim 6, wherein the polypeptide preferentially expresses in the soluble fraction of a prokaryotic host cell.

10. The polypeptide of claim 1, wherein the ST3GalI polypeptide and the ST6GalNAcI polypeptide each independently are from an animal selected from the group consisting of human, chimpanzee, pig, cow, dog, rat, mouse and chicken.

11. The polypeptide of claim 1, wherein the chimeric polypeptide has at least 90% identity with a polypeptide selected from the group consisting of the sequences shown in FIGS. 2A-E.

12. The polypeptide of claim 1, wherein the chimeric polypeptide is selected from the group consisting of the sequences shown in FIGS. 2A-E.

13. The polypeptide of claim 1, lacking one or more native N-linked glycosylation sites.

14. The polypeptide of claim 1, having one or more non-native N-linked glycosylation sites.

15. The polypeptide of claim 1, further comprising a tag polypeptide.

16. The polypeptide of claim 15, wherein said tag polypeptide is selected from the group consisting of a maltose binding protein (MBP) tag, a histidine tag, a Factor IS tag, a glutathione-S-transferase tag, a FLAG-tag, and a starch binding domain (SBD) tag.

17. A chimeric polypeptide comprising an N-terminal portion from a ST6GalNAcI polypeptide and a C-terminal portion from a ST3 GalI polypeptide, wherein the polypeptide catalyzes the transfer of a sialic acid moiety from a donor moiety to an acceptor moiety.

18. The polypeptide of claim 17, wherein the N-terminal sequence from the ST3 GalI polypeptide portion lacks one or more amino acid residues from the signal peptide.

19. The polypeptide of claim 18, wherein the N-terminal sequence from the ST3 GalI polypeptide portion lacks one or more amino acid residues from the transmembrane domain.

20. The polypeptide of claim 19, wherein the N-terminal sequence from the ST3GalI polypeptide portion lacks one or more amino acid residues from the stem domain.

21. The polypeptide of claim 17, wherein the polypeptide has at least 90% identity to the sequence shown in FIG. 2E (ST6GalNAc-ST3 Gal-1 fusion).

22. A modified ST6GalNAcI polypeptide having one or more modified native N-linked glycosylation sites.

23. The polypeptide of claim 22, lacking one or more native N-linked glycosylation sites.

24. The polypeptide of claim 22, having one or more non-native N-linked glycosylation sites.

25. The polypeptide of claim 22, wherein the polypeptide has at least 90% identity to the sequences shown in FIG. 2E-F (ST6GalNAc N-linked glycosylation mutants).

26. A nucleic acid encoding a polypeptide of claim 1, claim 17, or claim 22.

27. The nucleic acid of claim 26, said nucleic acid further comprising a promoter/regulatory sequence operably linked thereto.

28. An expression vector comprising the nucleic acid of claim 26.

29. A prokaryotic host cell comprising the nucleic acid of claim 28.

30. The prokaryotic host cell of claim 29, wherein the host cell is a strain selected from the group consisting of Escherichia coli, Pseudomonas, Bacillus, Ralstonia, and Caulobacter.

31. A method of producing a modified ST6GalNAc polypeptide, the method comprising growing the recombinant cell of claim 30 under conditions suitable for expression of the chimeric polypeptide.

32. A method of catalyzing the transfer of a sialic acid moiety to an acceptor moiety comprising incubating the modified ST6GalNAc polypeptide of claim 1, claim 17, or claim 22 with a sialic acid moiety and an acceptor moiety, wherein the polypeptide mediates the covalent linkage of the sialic acid moiety to the acceptor moiety, thereby catalyzing the transfer of a sialic acid moiety to an acceptor moiety.

33. A method of catalyzing the transfer of a sialic acid moiety to an acceptor moiety comprising incubating the modified ST6GalNAc polypeptide of claim 1, claim 17, or claim 22 with a cytidinemonophosphate-sialic acid (CMP-NAN) sialic acid donor and an asialo bovine submaxillary mucin acceptor moiety, wherein the polypeptide mediates the transfer of the sialic acid moiety from the CMP-NAN sialic acid donor to the asialo bovine submaxillary mucin acceptor, thereby catalyzing the transfer of a sialic acid moiety to an acceptor moiety.


SEQUENCE LISTING (114 sequences)

SEQ ID NO: 1 - DNA, 1800 bp, Homo sapiens
  human wild-type GalNAc alpha-2,6-sialyltransferase I, alpha-N-acetylgalactosaminide alpha-2,6-sialyltransferase 1, sialyltransferase 7A (ST6GalNAcI, SIAT7A)
atgaggtcctgcctgtggagatgcaggcacctgagccaaggcgtccagtggtccttgcttctggctgtcctggtcttctttctcttcgccttgccctcttttattaaggagcctcaaacaaagccttccaggcatcaacgcacagagaacattaaagaaaggtctctacagtccctggcaaagcctaagtcccaggcacccacaagggcaaggaggacaaccatctatgcagagccagtgccagagaacaatgccctcaacacacaaacccagcccaaggcccacaccaccggagacagaggaaaggaggccaaccaggcaccgccggaggagcaggacaaggtgccccacacagcacagagggcagcatggaagagcccagaaaaagagaaaaccatggtgaacacactgtcacccagagggcaagatgcagggatggcctctggcaggacagaggcacaatcatggaagagccaggacacaaagacgaccaaaggaaatgggggccagaccaggaagctgacggcctccaggacggtgtcagagaagcaccagggcaaagcggcaaccacagccaagacgctcattcccaaaagtcagcacagaatgctggctcccacaggagcagtgtcaacaaggacgagacagaaaggagtgaccacagcagtcatcccacctaaggagaagaaacctcaggccaccccaccccctgcccctttccagagccccacgacgcagagaaaccaaagactgaaggccgccaacttcaaatctgagcctcggtgggattttgaggaaaaatacagcttcgaaataggaggccttcagacgacttgccctgactctgtgaagatcaaagcctccaagtcgctgtggctccagaaactctttctgcccaacctcactctcttcctggactccagacacttcaaccagagtgagtgggaccgcctggaacactttgcaccaccctttggcttcatggagctcaactactccttggtgcagaaggtcgtgacacgcttccctccagtgccccagcagcagctgctcctggccagcctccccgctgggagcctccggtgcatcacctgtgccgtggtgggcaacgggggcatcctgaacaactcccacataggccaggagatagacagtcacgactacgtgttccgattgagcggagctctcattaaaggctacgaacaggatgtggggactcggacatccttctacggctttaccgccttctccctgacccagtcactccttatattgggcaatcggggtttcaagaacgtgcctcttgggaaggacgtccgctacttgcacttcctggaaggcacccgggactatgagtggctggaagcactgcttatgaatcagacggtgatgtcaaaaaaccttttctggttcaggcacagaccccaggaagcttttcgggaagccctgcacatggacaggtacctgttgctgcacccagactttctccgatacatgaagaacaggtttctgaggtctaagaccctggatggtgcccactggaggatataccgccccaccactggggcccttctgctgctcactgcccttcagctctgtgaccaggtgagtgcttatggcttcatcactgagggccatgagcgcttttctgatcactactatgatacatcatggaagcggctgatcttttacataaaccatgacttcaagctggagagagaagtctggaagcggctacacgatgaagggataatccggctgtaccagcgtcctggtcccggaactgccaaagccaagaac

SEQ ID NO: 2 - protein, 600 aa, Homo sapiens
  human wild-type GalNAc alpha-2, 6-sialyltransferase I, alpha-N-acetylgalactosaminide alpha-2,6-sialyltransferase 1, sialyltransferase 7A (ST6GalNAcI, SIAT7A)
MRSCLWRCRHLSQGVQWSLLLAVLVFFLFALPSFIKEPQTKPSRHQRTENIKERSLQSLAKPKSQAPTRARRTTIYAEPVPENNALNTQTQPKAHTTGDRGKEANQAPPEEQDKVPHTAQRAAWKSPEKEKTMVNTLSPRGQDAGMASGRTEAQSWKSQDTKTTKGNGGQTRKLTASRTVSEKHQGKAATTAKTLIPKSQHRMLAPTGAVSTRTRQKGVTTAVIPPKEKKPQATPPPAPFQSPTTQRNQRLKAANFKSEPRWDFEEKYSFEIGGLQTTCPDSVKIKASKSLWLQKLFLPNLTLFLDSRHFNQSEWDRLEHFAPPFGFMELNYSLVQKVVTRFPPVPQQQLLLASLPAGSLRCITCAVVGNGGILNNSHIGQEIDSHDYVFRLSGALIKGYEQDVGTRTSFYGFTAFSLTQSLLILGNRGFKNVPLGKDVRYLHFLEGTRDYEWLEALLMNQTVMSKNLFWFRHRPQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 3 - DNA, 1641 bp, Mus musculus
  mouse wild-type GalNAc alpha-2, 6-sialyltransferase I, alpha-N-acetylgalactosaminide alpha-2, 6-sialyltransferase 1, sialyltransferase 7A (ST6GalNAcI, SIAT7A)
atgctactagtaaatcagtcacaccaaggcttcaataaggaacacacaagcaagatggtaagcgctattgttttatatgtgcttttggcggcggcggcgcattctgcctttgcggcggatccaagggcaaaagattccaggtgccagttcatatggaagaatgacgcaagtgcccaggagaatcaacaaaaggctgagccccaagtacccatcatgacactgtcacccagagttcacaacaaggagtcaacctctgtcagttcaaaggacctgaagaaacaggagagagaggcagtccaaggagagcaagctgaggggaaggagaagaggaagttagagaccataaggccagcaccagagaatcctcagagcaaggcagagcctgctgcaaagacgcctgtgtcagaacacctggacaaactacccagaactccaggagcactgtcaacaaggaagacaccaatggccacaggagctgtcccagctaagaagaaagtggtccaggctaccaaatcccctgcctcttccccacaccccaccacacgaagaaggcaaaggctgaaggcctctgagttcaagtctgagcctcggtgggattttgaggaggaatatagcttggatatgagcagcctgcagacgaactgctctgcttctgtgaagatcaaggcctccaagtcaccatggctacagaatatctttctgcccaacatcactctgttcctggactctggacgcttcacccagagcgagtggaaccgtctagagcacttcgctcctccctttggcttcatggaactcaatcagtccctggtacagaaggtggtgacccgcttccctccagttcgccagcagcagctcctcctggccagcctccctactgggtactccaagtgtatcacctgtgctgtagtgggcaacgggggcatcctgaatgattcacgtgttggccgggagatagacagccatgactatgttttccgactgagtggagccgttattaaaggatatgaacaggatgtggggacccggacatccttctatggcttcactgctttctctctgacccagtctatcctcattttgggtagacgaggcttccagcatgtgcctctgggaaaggacgtccgatatctacacttcctggaaggcacccgggactatgagtggctggaagctatgtttttgaatcagaccttggcaaaaacccacctttcctggttcaggcataggcctcaggaagccttccggaatgccttggacttggatcgatacttgctgctgcacccagactttctccgttacatgaagaacaggtttctgaggtcaaagaccctggacactgcccattggagaatataccgccccaccactggtgccctcctgctgctcacagcccttcatctctgtgacaaggtcagcgcctatggcttcatcaccgagggccaccagcgcttctctgaccactactatgatacatcatggaaacggctcatcttttatatcaaccatgacttcaggttagagagaatggtgtggaagcggttgcatgatgaaggcatcatctggctctaccagcgtccacaaagtgacaaagcaaagaacggcggccgcgaacaaaaactcatctcagaagaggatctg

SEQ ID NO: 4 - protein, 547 aa, Mus musculus
  mouse wild-type GalNAc alpha-2, 6-sialyltransferase I, alpha-N-acetylgalactosaminide alpha-2, 6-sialyltransferase 1, sialyltransferase 7A (ST6GalNAcI, SIAT7A)
MLLVNQSHQGFNKEHTSKMVSAIVLYVLLAAAAHSAFAADPRAKDSRCQFIWKNDASAQENQQKAEPQVPIMTLSPRVHNKESTSVSSKDLKKQEREAVQGEQAEGKEKRKLETIRPAPENPQSKAEPAAKTPVSEHLDKLPRTPGALSTRKTPMATGAVPAKKKVVQATKSPASSPHPTTRRRQRLKASEFKSEPRWDFEEEYSLDMSSLQTNCSASVKIKASKSPWLQNIFLPNITLFLDSGRFTQSEWNRLEHFAPPFGFMELNQSLVQKVVTRFPPVRQQQLLLASLPTGYSKCITCAVVGNGGILNDSRVGREIDSHDYVFRLSGAVIKGYEQDVGTRTSFYGFTAFSLTQSILILGRRGFQHVPLGKDVRYLHFLEGTRDYEWLEAMFLNQTLAKTHLSWFRHRPQEAFRNALDLDRYLLLHPDFLRYMKNRFLRSKTLDTAHWRIYRPTTGALLLLTALHLCDKVSAYGFITEGHQRFSDHYYDTSWKRLIFYINHDFRLERMVWKRLHDEGIIWLYQRPQSDKAKNGGREQKLISEEDL

SEQ ID NO: 5 - DNA, 1698 bp, Gallus gallus
  chicken wild-type GalNAc alpha-2, 6-sialyltransferase I, alpha-N- acetylgalactosaminide alpha-2, 6-sialyltransferase 1, ST6 (alpha-N-acetyl-neuraminyl-2, 3-beta-galactosyl-1,3)-N-acetylgalactosaminide alpha-2, 6-sialyltransferase 1 (ST6GalNAcI)
atggggtttttaatcagaaggcttcctaaagattccagaatattccgttggctccttattttaacagtcttttccttcatcattactagttttagcgccttgtttggcatggagaaaagcattttcaggcagctcaagatttaccaaagcattgcacatatgctacaagtggacacccaagatcagcaaggttcaaactattctgctaatgggagaatttcaaaggttggtttggagagagacattgcatggctcgaactgaatactgctgtgagtacaccaagtggggaagggaaggaagagcagaagaaaacagtgaaaccagttgccaaggtggaagaagccaaggagaaagtgactgtgaaaccattccctgaggtgatggggatcacaaatacaacagcatcaacagcctctgtggtggagagaacaaaggagaaaacaacagcgagaccagttccaggggtgggggaagctgatgggaagagaacaacgatagcacttcccagcatgaaggaagacaaagagaaggcgactgtgaaaccatcctttgggatgaaggtagctcatgcaaacagcacatccaaagataaaccaaaggcagaagagcctcctgcatcagtgaaagccataagacctgtgactcaggctgccacagtgacagagaagaagaaactgagggctgctgacttcaagactgagccacagtgggattttgatgatgagtacatactggatagctcatctccagtatcgacctgctctgaatcagtgagagccaaggctgccaagtctgactggctgcgagatcttttcctgccgaacatcacactcttcatagacaagagttacttcaatgtcagtgagtgggaccgcctggagcattttgcacctccctatggcttcatggagctgaattactcactggtagaagaagtcatgtcacggctgcctccaaatccccaccagcagctgctcctggccaacagtagcagcaacgtgtcaacgtgcatcagctgtgctgttgtggggaatggagggatattgaataactctggaatgggccaggagattgactcccatgactatgtgttccgggtgagcggggctgtaatcaaaggttacgaaaaggatgtgggaacaaaaacctccttctacggattcacagcgtactccctggtgtcctctctccagaacttgggacacaaagggttcaagaagatcccacaggggaagcatatcagatacattcacttcctggaggcagttagagactatgagtggctgaaggctcttctgttggacaaggatatcaggaaaggattcctgaactactatgggcgaaggccccgggagagattcgatgaagatttcacaatgaataagtacctggtagctcaccctgatttcctcagatacttgaaaaacaggttcttaaaatctaaaaatctgcaaaagccctactggcggctgtacagacccacaacaggagccctcctgctgctgactgccctgcatctctgtgaccgggtgagtgcctatggctacatcacagaaggtcaccagaagtactcggatcactactatgacaaggagtggaaacgcctggtcttctacgttaaccatgacttcaacttggagaagcaggtgtggaaaaggcttcatgatgagaacatcatgaagctctaccagagatcc

SEQ ID NO: 6 - protein, 566 aa, Gallus gallus
  chicken wild-type GalNAc alpha-2, 6-sialyltransferase I, alpha-N-acetylgalactosaminide alpha-2, 6-sialyltransferase 1, ST6 (alpha-N-acetyl-neuraminyl-2,3-beta- galactosyl-1,3)-N-acetylgalactosaminide alpha-2, 6-sialyltransferase 1 (ST6GalNAcI)
MGFLIRRLPKDSRIFRWLLILTVFSFIITSFSALFGMEKSIFRQLKIYQSIAHMLQVDTQDQQGSNYSANGRISKVGLERDIAWLELNTAVSTPSGEGKEEQKKTVKPVAKVEEAKEKVTVKPFPEVMGITNTTASTASVVERTKEKTTARPVPGVGEADGKRTTIALPSMKEDKEKATVKPSFGMKVAHANSTSKDKPKAEEPPASVKAIRPVTQAATVTEKKKLRAADFKTEPQWDFDDEYILDSSSPVSTCSESVRAKAAKSDWLRDLFLPNITLFIDKSYFNVSEWDRLEHFAPPYGFMELNYSLVEEVMSRLPPNPHQQLLLANSSSNVSTCISCAVVGNGGILNNSGMGQEIDSHDYVFRVSGAVIKGYEKDVGTKTSFYGFTAYSLVSSLQNLGHKGFKKIPQGKHIRYIHFLEAVRDYEWLKALLLDKDIRKGFLNYYGRRPRERFDEDFTMNKYLVAHPDFLRYLKNRFLKSKNLQKPYWRLYRPTTGALLLLTALHLCDRVSAYGYITEGHQKYSDHYYDKEWKRLVFYVNHDFNLEKQVWKRLHDENIMKLYQRS

SEQ ID NO: 7 - DNA, 1704 bp, Pan troglodytes
  chimpanzee wild-type alpha-2, 6-sialyltransferase, sialyltransferase 7A (ST6GalNAcI, SIA7A)
atgaggtcctgcctgtggagatgcaggcacctgagccaaggcgtccagtggtccttgcttctggctgtcctggtcttctttctcttcgccttgccctcttttattaaggagcctcaaacaaagccttccagagagccagcgccagagaacaatgccctcaacacacaaacccagcccaaggcccacaccgccggagacagaggaaaggaggccaaccaggcaccgccggaggagcaggacaaggtgccccacacagcacagagggcagcatggaagagcccagaaaaagagaaaaccatggtgaacacactgtcacccagagggcaagatgcagggatggcctctggcaggacacaggcacaatcatggaagagccaggacacaaagacgacccaaggaaatgggggccagaccaggaagctgatggcctccaggacagtgtcagagaagcaccagggcaaagcggcgaccacagccaagacgctcattcccaaaagtcagcacagaatgctggctcccacaggagcagtgtcaacaaggacgagacagaaaggagtgaccacggcagtcatcccacctaaggagaagaaacctcaggccaccccaccccctgcccctttccagagccccacgacgcagagaaaccaaagactgaaggccgccaacttcaaatctgagcctcggtgggattttgaggaaaaatacagcttcgaaataggaggccttcagacgacttgccctgactctgtgaagatcaaagcctccaagtcgctgtggctccagaaactctttctgcccaacctcactctcttcctggactccagacacttcaaccagagtgagtgggaccgcctggaacactttgcaccaccctttggcttcatggagctcaactactccttggtgcagaaggtcgtgacacgcttccctccagtgccccagcagcagctgctcctggccagcctccccgctgggagcctccggtgcatcacctgtgccgtggtgggcaacgggggcatcctgaacaactcccacatgggccaggagatagacagtcatgactacgtgttccgattgagcggagttctcattaaaggctacgaacaggatgcggttgatcggacatccttctacggctttaccgccttctccctgacccagtcactccttatattgggcaatcggggtttcaagaacgtgcctctcgggaaggacgtccgctacttgcacttcctggaaggcacccgggactatgagtggctggaagcactgcttatgaatcagacggtgatgtcaaaaaaccttttctggttcaggcacagaccccaggaagcttttcgggaagccctgcacatggacaggtacctgttgctgcacccagactttctccgatacatgaagaacaggtttctgaggtctaagaccctggatggtgcccactggaggatataccgccccaccactggggccctcctgctgctcactgcccttcagctctgtgaccaggtgagtgcttatggcttcatcaccgagggccatgagcgcttttctgatcactactatgatacatcatggaagcggctgatcttttacacaaaccatgacttcaagctggagagagaagtctggaagcggctacacgatgaagggataatccggctgtaccagcgtcctggtcccggaactgccaaagccaagaactga

SEQ ID NO: 8 - protein, 567 aa, Pan troglodytes
  chimpanzee wild-type alpha-2, 6-sialyltransferase, sialyltransferase 7A (ST6GalNAcI, SIA7A)
MRSCLWRCRHLSQGVQWSLLLAVLVFFLFALPSFIKEPQTKPSREPAPENNALNTQTQPKAHTAGDRGKEANQAPPEEQDKVPHTAQRAAWKSPEKEKTMVNTLSPRGQDAGMASGRTQAQSWKSQDTKTTQGNGGQTRKLMASRTVSEKHQGKAATTAKTLIPKSQHRMLAPTGAVSTRTRQKGVTTAVIPPKEKKPQATPPPAPFQSPTTQRNQRLKAANFKSEPRWDFEEKYSFEIGGLQTTCPDSVKIKASKSLWLQKLFLPNLTLFLDSRHFNQSEWDRLEHFAPPFGFMELNYSLVQKVVTRFPPVPQQQLLLASLPAGSLRCITCAVVGNGGILNNSHMGQEIDSHDYVFRLSGVLIKGYEQDAVDRTSFYGFTAFSLTQSLLILGNRGFKNVPLGKDVRYLHFLEGTRDYEWLEALLMNQTVMSKNLFWFRHRPQEAFREALHMDRYLLLHPDFLRYMKNRFLRSKTLDGAHWRIYRPTTGALLLLTALQLCDQVSAYGFITEGHERFSDHYYDTSWKRLIFYTNHDFKLEREVWKRLHDEGIIRLYQRPGPGTAKAKN

SEQ ID NO: 9 - DNA, 2956 bp, Rattus norvegicus
  rat wild-type alpha-2,6-sialyltransferase, sialyltransferase 7A (ST6GalNAcI, SIA7A)
caacttccatgcccatattacatcttctccttcacacaaaatagggaacagggaaggaaggaaatcagcaaagaatcttcatctaaatgtcctgggagaggctgcctggatcacagtgtggagatgaggtatcaaagcccggatactgatgagtggtatctgcctgtggagtggtccagctgcggggccgaggtgcagcgagccacagggtcgatccatagcgccatggcctgcatcctcaagaggaagcctgcgcttgcggtgagcttcatagccctgtgcatcttgctgctagccatgcgccttgccaatgacgtgactttccctttgctcctgaactgctttggacaacctaaaaccaaatggatccctttgtcctacacactcaggcagcctcttcaaactcactatggatacataaacgtgaggacccaagagcctttgcagctgaactgtaaccattgtgccgtcgtgtcgaactcgggtcagatggttggacagaaggtgggggaagagatagaccgtgcgtcctgcatctggagaatgaacaatgcccccaccaagggtttcgaggaagatgtcggctacatgacgatggtccgagtggtgtcacacaccagcgtccctctcttgctgaaaaacccggactattttttcaaggaggcgagcacaaccatttacgtcatctggggccctttccgcaacatgaggaaggacgggaacgggatcgtgtacaacatgctaaagaagacggtcgacgcctacccagacgcgcagatctacgtgaccacggagcagcggatgacgtactgcgacggggtgtttaaggatgaaactgggaaagacagagtccagtctggctcctatctcagcaccgggtggttcacctttatcctggccatggacgcctgctacagtatccacgtctacgggatgataaatgagacctactgcacgacagaagggtatagaaaagtcccctatcattactatgaacaagggaaggacgagtgtaacgagtatcttctccatgaacatgccccatacgggggacacaggttcatcaccgagaagaaagtgtttgccaagtgggccaagaaacacagaatagtatttacacacccaaactggacagtgtcctgatgctgtgcttcctgaccgtgacactgcagctgtgaggaagagaggttgatggtggtgacagtgcgggcaggatgctcatggtgaccatggtcctggacattctctgtggggacactgactctgctagtaatttgaactttggcattctgtgggagggggtcatgttcattctggtccttctggtggttcagccctgcgaattgcactttaaaacttacctacaactgaaatgaaggctgtgactaaagaaaacacgaagattacctttcaagatggctgctcagaattctttaacagaggcttccaggactcacgattcagcctgctcaggcaaggttactgagtgcatgggctgctccccatcttggaaaatggtggactgtcataatacactgtccccaagcaattctttcagggtagagatatcttgccaccgttaccagttaccctgatagaggggaggtatctctttctgctctgccatctttcttccatatgtctctcatctaaagcctcaggccacctaactaaatgtagcaatgaaggcattctgtaattcctgcatatcaggattgctggactgtctcagagacttaattcaaaatgtatcaaatagcaaatattgacaatttttcacttggacagctgtgctctcactactcaaaacaatgtaactaaacatattactacaattacctacattaatgtttagttatttttggttgagtgctaaacattaacaggttagatatactgctacactgtaataacacatactgtgaaagcatttcttgaaaataaccaaagggttcctcactcttcttcaacactcagtaccaagaaatgagatgctctttgaaaccacaaaacaggggaaagcaagacatatttttgcatcaatttgtatcctatcatattcataatatatatttttaaattcaaatttctattttataagctgaaggtgtacctctctccatagacattatcaatgtcaagtgccaaacggtagatatggaaccttctttatatcatcaaacccagcccaagcttttgccttcatagggctgattgtcccttaggttgccgtatgttctgctagaaaagtaaaatggtcatgagcttcattcccaaagtgcttcgctatgcttcacataggctatgtaccacgtgacgcccatggccacctccctggactattgcctgcacattctgccatccccagcagctgatctgcattgttggctgtgttcatgtgtcccaggtcgctctagttcagaatataggtatagaatagcatttgtgtaaatcagtaaacgactgagttctctgccaagcacagcatagcataccgagattatgcaaaatggccacaaggacggccgtgtcatagatcagtaccctataagcttagataccttcaatgtttttgcttctgttcgatggctctctgttccagagctcggggacaatggctaggaatggtagcagagaaatacagtttccaggtctagacctgagacggctttcttctctgtagactcaatgatccttaaatattagaggttgattttctgagacacttccaagcatccataaataccttggcacaatgattcaaagctaaaaccaagctgcattgagtgttttagagctggaaatgacaaaacacctcacctgtgccacctcgacagggctttcctacgattaagggctatgctaactcattaagatgtgcatctttcctaccctctgcacccattttgatatcccacaccccgacccataagctttgcccagccccactcgtgcc

SEQ ID NO: 10 - protein, 519 aa, Rattus norvegicus
  rat wild-type alpha-2,6-sialyltransferase, sialyltransferase 7A (ST6GalNAcI, SIA7A)
MENRCRGLSQGQTFLLLTGLMLLFILPSVVKEPSTRVSRQFIEDNESSLQGVPQKPAPQGPIVTLTPTVHNKKTTSVRTKWVELQKQDRATARGERGEGVEKKLQAIRLAPENPKGKAEPEVKTPASKHLDKLPRATGALSTRKTQMATGAAPAKKKVVQPTPTPASFPHLTTQRRQRLKASDFKSEPRWDFEEEYSLDGGSLQTLPWFLKITVSHSPWVQNIFLPNITLFLDSGRFNQSEWYRLEHFTPPFGFMELNQSLVQKVVSRFPPVPQQQLLLASLPTRNLTCITCAVVGNGGILNNSRMGQEIDSHDYVFRLSGTVIKGYEQDVGTRTSFYGFTAFSLGQSILNLGSSRFSACAFLGKDVRYLHFLEGTRDYEWLEAMFLNRTMANTKLYWFRHRPQEAFREALDLDRYFLVHPDFLRYMKNRFLRSKTLDTAHWRLYRPTTGALLLLTALHLCDKVSAYGFITQGHERFSDHYYDTSWKRLIFYINHDFALERTVWKRLHDEGIIQLYQRP

SEQ ID NO: 11 - DNA, 2104 bp, Bos taurus
  cow (predicted) similar to wild-type GalNAc alpha-2,6-sialyltransferase I (ST6GalNAcI), LOC618655
atggcgacatgtcccgcctgcggccgccttcgttccctcctgaggccaggctcccgcaagaatgagcatcaacagaacattaaggaaaggtccccggagttactgcaaaatgctacgtctcaggcacccaccccgagaaggagggcgaccacccatgtggagtcagtacaggggacccgcactcgggacacacaccccaaggccacaaccctcacggcggaccagcgcagaagagtgcagaccagcaaggcccgtgccgaggagccgggcagagtgcctacccccatcggaaaggcagcaccgcagacgcaggcgagcaaggacacccgggcggacacgctgcccccgatggctggaggtgggggggtggcctccagcaggaccgaggcgccatcactgaacagtcagaacccaaggatgaccaaaggatctggggaccggaaggcaaggcccacaggccccagagcggtgcccacaaagctgcgggacagcccgagccctgccacccagagaagccagaagctgaaggccaccaacttcaagtctgagccccagtgggattttgaggaagaatacagtctggaggtcggtggcctgcagacggggacccgcactcgggacacacaccccaaggccacaaccctcacggcggaccagcgcagaagagtgcagaccagcaaggcccgtgccgaggagccgggcagagtgcctacccccatcggaaaggcagcaccacagacgcaggcgagcaaggacacccgggcggacacgctgcccccgatggctggaggtgggggggtggcctccagcaggaccgaggcgccatcactgaacagtcagaacccaaggatgaccaaaggatctggggaccggaaggacagagcccagcccacccggtcctcggcccctctccagagccctgccacccagagaagccagaagctgaaggccaccaacttcaagtctgagccccagtgggattttgaggacgaatacagtctggaggtcggcggcctgcagacgacctgccctgactcggtgaagatcaaagcctccaagtcaccctggctccggactctctttctgcccaacctcaccctctttctggactccggacacttcaaccagagcgagtgggaccgcctggagcacttcgctccgcccttcggcttcatggagctcaatttctccttggtgcagaaggtcgtggcacgcttccccccggtgccccagcagcagctgctcctggccagcctccccgccgggagctcccggtgcatcagctgtgccgtggtgggcaacgggggcatcctgaacaactcccacgtgggcccagagatagacagccacgactacgtgttccggctgagtggagccatcattaaaggctatgaacacgatgtgggtactcggacctccttctacggctttaccgccttctctctgacccagtcactccttatactgggcagtcggggtttcccgcatgcgcctctggggcaggacgtccgctacctgcacttcctggaaggcacccgggactatgagtggctggaagcactgcttctgaatcggaccttgacatcaaggaacctttcctggttcaggcgcagaccccaggaggccttccaggaagccttgcaactggacagatacctgttgctgcacccagacctgctccgatacatgaagaacagattcctgaggtctaagactctgaacactgcccactggagaatataccggcccaccacgggggcccttctgctgctcactgccctccagctctgtgaccaggtgagtgcctacgggttcatcactgagggccacgaacgcttctctgaccactactacgataagtcctggaaacgaacgatcttttacaccaaccatgacttcaagttggagagggcgctctggaagcggctacatgatgaaggtattatccggctgtaccaacgtcctataacttccaaaccgaccatctgaagggagcctgggctaccgaagtcccttcagctattctaaggctcagagcacagtgggagaccagagactcgtctgccatcttcccagcgcctgcaccaggctctgagcccctcca

SEQ ID NO: 12 - protein, 662 aa, Bos taurus
  cow (predicted) similar to wild-type GalNAc alpha-2,6-sialyltransferase I (ST6GalNAcI)
MATCPACGRLRSLLRPGSRKNEHQQNIKERSPELLQNATSQAPTPRRRATTHVESVQGTRTRDTHPKATTLTADQRRRVQTSKARAEEPGRVPTPIGKAAPQTQASKDTRADTLPPMAGGGGVASSRTEAPSLNSQNPRMTKGSGDRKARPTGPRAVPTKLRDSPSPATQRSQKLKATNFKSEPQWDFEEEYSLEVGGLQTGTRTRDTHPKATTLTADQRRRVQTSKARAEEPGRVPTPIGKAAPQTQASKDTRADTLPPMAGGGGVASSRTEAPSLNSQNPRMTKGSGDRKDRAQPTRSSAPLQSPATQRSQKLKATNFKSEPQWDFEDEYSLEVGGLQTTCPDSVKIKASKSPWLRTLFLPNLTLFLDSGHFNQSEWDRLEHFAPPFGFMELNFSLVQKVVARFPPVPQQQLLLASLPAGSSRCISCAVVGNGGILNNSHVGPEIDSHDYVFRLSGAIIKGYEHDVGTRTSFYGFTAFSLTQSLLILGSRGFPHAPLGQDVRYLHFLEGTRDYEWLEALLLNRTLTSRNLSWFRRRPQEAFQEALQLDRYLLLHPDLLRYMKNRFLRSKTLNTAHWRIYRPTTGALLLLTALQLCDQVSAYGFITEGHERFSDHYYDKSWKRTIFYTNHDFKLERALWKRLHDEGIIRLYQRPITSKPTI

SEQ ID NO: 13 - DNA, 3057 bp, Canis lupus familiaris
  dog (predicted) similar to wild-type GalNAc alpha-2,6-sialyltransferase I (ST6GalNAcI), LOC483334
atgaagatccgcctgtgtagactcagtcatctggaccaaactacacaatggttcctgctttcggctgtgttgaccttctttgtcttcatcctgccctcctttgttaaggagcccaataccaaacctttcaggcatcagcacatacgcaatgaggagaggtccccagagtcactccatgaggtgacagggcaggcccccacagcgggagacaggacaatggccccggcggtggcatcagcgcaagagaagagcgtgtgggccccccaggccctggccacagcctacgctgcccaggaggacaccagagcagaggccctcgaggccacctcagctcagcccacctccatgaggggtcaggagaggaaggacaccatgccggacacgctgcccccgagggctcaggacaagggggtggcgtctgacaggacagggctcccatcagtgatgagtcaggacactaggacaatcaaaggaaggcgggaccagaaggaggagccgacagccaccaggaaggtgaccccagggcctcagagcaaggcgcggaccacccggcgaacacctgtgccagaaagccaggccaaggcgctgaccacgccaggagcagggccgatggggaggacgaggaaaggagccaccacagcggctgccccgcacagggacacagcccgggccacgccaccctccacctcttcccagggccgcaccacccggagaagcccgagtctgcgggctgccaacttcaagtctgagccccggtgggatttcgaggaacagtacagctttgacacggaggccctgcagccgacctgcccggactcagtgaaggtcaaggcctccaagtcaccctggctccagaatctcttcctggccaacctcaccctcttcctggactccagccgcttcagccagagagagtgggaccgtctagaacacttcgcaccgcccttcggcttcatggagctcaatcagtccctggtgcaaaaaggggtgacccgcttccctccggtgccccagcagcagctgctccgggccagcctccccgccgggagctcccagtgcatcacctgcgccgtggtggggaacgggggcatcctgaacgactcgcgcgtgggccaggagatagacggccacgatcacgtgttccggttgagcggagctgtcactaagggctacgagcaggatgtgggcacccggacgtccttctacggcttcaccgccttctccataacgcagtcactgctcgccttgggcggtcggggcttcccgcacgtccctctggggaaggatgtccgctacctgcatttcctggaaggcacccgagactacgagtggctggaagcgctgcttctgaatcagaccctggtgaaaagtagcctttcctggttcaggcgcaggccccaggaggctttccgggataccttgcagttggacaggtacctgttgctacacccagactttatgcgatacatgaagaacaggtttttgaggtcaaagaccctgaacaatatccactggagaatataccgtcccaccactggggccctcctgctgctcacggcccttcatctctgtgaccaggtgagcgcctatggcttcatcaccgagggccacgagcgcttctctgatcactactatgataagtcctggaaacgaacgatcttttataccaaccatgacttcccgttagagagaacactctggaagcggctgcacgatgaaggtataatccggctgtaccagcgtcccataatttccaaaccaaagatggttcagaccaggccccaggcccccccagactcaccaggggacactggcaccaaggaggagaacccgcactgccgacgcccacttgacctggccatccagaggcacccccacttccgtgccctgttcgatctctccactccagtgctgctatcggggagcctcttcactcaggagctctgggacagcctgagccagcacaaagccccctacggctggcagggcctgtcccaccaagccattaactccaccctgagcctcctgaacggctccgagagctcccagctgttcgccacctccaggaagccgctttcaagctgcattcggtgcgcggtggtggggaacgggggcattctgaacggttcccgccagggtctgaacatcgacgcccatgactacgtgttcagacagaccctgaagacggagcgagggggccgcgtgcctgccaggagggggcccccgggccggatgtcccctgaggtccggagatgtggggatgggctggatggggacagcggtggacgtggtcagcacagctacccatttccagacggcaacgtgtctgtgcaaggccaccccgcagctaagatcatcaaagtccagccccctctgaccacaccagccctagggctgcctcccccttccctgagctgccttcctgacgacctgcgctacatcttcatcccctcggacatccgcgattatgtgatgctgagatcggccattctgggcgtgcctgtccccgagggccctgataaaggcgacaggtccccttcccacagccccgccacctcggcctcctacatcgggcccgttcggggcatcccctcctacatcgcgccctatgtgccccgcttcctgaaggagccccccttcttccagcccccgaccgtcctggtgggcgggaagccgcacgtggtgcccaccgtgctggccgccaccttctacacgcccttcttccgctattacgcccagccctggccctaccgcccgcaccgtaagaggccttccctctcaggggcccgcagcagcagaggactggtgtgggtggaacctaacctgatctgggacaacccctacaaggggcctgggtcacgcttggacctggaactcggcctctaccctgttagtgcctatggattcatcacaagcaactactggaaattttctgaccactatttcgagcgagttaagaagccactgatattctatgtcaaccacgacctgtccctggaagcctccctgtggagggacctgcacaaggccggcatccttcaactgtaccagcgctga

SEQ ID NO: 14 - protein, 1018 aa, Canis lupus familiaris
  dog (predicted) similar to wild-type GalNAc alpha-2,6-sialyltransferase I (ST6GalNAcI)
MKIRLCRLSHLDQTTQWFLLSAVLTFFVFILPSFVKEPNTKPFRHQHIRNEERSPESLHEVTGQAPTAGDRTMAPAVASAQEKSVWAPQALATAYAAQEDTRAEALEATSAQPTSMRGQERKDTMPDTLPPRAQDKGVASDRTGLPSVMSQDTRTIKGRRDQKEEPTATRKVTPGPQSKARTTRRTPVPESQAKALTTPGAGPMGRTRKGATTAAAPHRDTARATPPSTSSQGRTTRRSPSLRAANFKSEPRWDFEEQYSFDTEALQPTCPDSVKVKASKSPWLQNLFLANLTLFLDSSRFSQREWDRLEHFAPPFGFMELNQSLVQKGVTRFPPVPQQQLLRASLPAGSSQCITCAVVGNGGILNDSRVGQEIDGHDHVFRLSGAVTKGYEQDVGTRTSFYGFTAFSITQSLLALGGRGFPHVPLGKDVRYLHFLEGTRDYEWLEALLLNQTLVKSSLSWFRRRPQEAFRDTLQLDRYLLLHPDFMRYMKNRFLRSKTLNNIHWRIYRPTTGALLLLTALHLCDQVSAYGFITEGHERFSDHYYDKSWKRTIFYTNHDFPLERTLWKRLHDEGIIRLYQRPIISKPKMVQTRPQAPPDSPGDTGTKEENPHCRRPLDLAIQRHPHFRALFDLSTPVLLSGSLFTQELWDSLSQHKAPYGWQGLSHQAINSTLSLLNGSESSQLFATSRKPLSSCIRCAVVGNGGILNGSRQGLNIDAHDYVFRQTLKTERGGRVPARRGPPGRMSPEVRRCGDGLDGDSGGRGQHSYPFPDGNVSVQGHPAAKIIKVQPPLTTPALGLPPPSLSCLPDDLRYIFIPSDIRDYVMLRSAILGVPVPEGPDKGDRSPSHSPATSASYIGPVRGIPSYIAPYVPRFLKEPPFFQPPTVLVGGKPHVVPTVLAATFYTPFFRYYAQPWPYRPHRKRPSLSGARSSRGLVWVEPNLIWDNPYKGPGSRLDLELGLYPVSAYGFITSNYWKFSDHYFERVKKPLIFYVNHDLSLEASLWRDLHKAGILQLYQR

SEQ ID NO: 15 - DNA, 2911 bp, Homo sapiens
  human wild-type ST3 beta-galactoside alpha-2, 3-sialyltransferase 1, transcript variant 1 (ST3GalI, ST3O), sialyltransferase 4A (SIAT4A, SIATFL), CMP-N-acetylneuraminate- beta-galactosamide-alpha-2,3-sialyltransferase, Gal-NAc6S, MGC9183
ctccagcctccgccggcggagcccactatgccagacagtttcgacactttgcaaagacaaagagccctagaccggagggaggaggaggaggaagaagagcggagagagaaggaagaggcgatgtgagctgggaagggggcaagtgtccgggacacccacacccctgtattctcctccgaaccccttcatgcccaaatcccggaaactccagcgtgtctccagccgtgttggtaccattttcagatttcatcttcctaaactggaaatgtcaatgagaggaaattaacacccccaagagctgcagtgagcaaatgcattgagcttgggtcaggacaattccatttggggaccagagatggacggtcactcagcctatggagatgaagaaactgaggttcagagaggttaagagactccactgaggtcacacagccgatgacagacaaccttctgtgccttcatcaagctggttgtgtacccaccatgtccctggcgacaggatgggaaagaaaaagccctaattaaggatcgtcagaaaccacagttggaggaggacggcagagacagtttccctccccgctataccaacacccttccttcgaggtcctcgctcctgagggaccctggactgtcacagagattaatgaccccttatcttctttggatgtgaaaggaaatcactggttaaagcttgatcgagagacattatcagctctttaaggattgcagaagaataggctactttattttctgaaaaggtaaatatatgcaagcaaagccaacatgccacgaatggcgttggtctaccacacagccgtgtctgggacacagttgggggtcatcccccagcaggagtgaagtcgagcttagcggcccttgtgtcctcccttggaattcctgccatcccttttgattgagcctccacctctgggatttttcttccatttttctcctctcttaggagggagttcctgctacccatcgtgggaggccaccatcaggactgcgaagatggtgaccctgcggaagaggaccctgaaagtgctcaccttcctcgtgctcttcatcttcctcacctccttcttcctgaactactcccacaccatggtggccaccacctggttccccaagcagatggtcctggagctctccgagaacctgaagagactgatcaagcacaggccttgcacctgcacccactgcatcgggcagcgcaagctctcggcctggttcgatgagaggttcaaccagaccatgcagccgctgctgaccgcccagaacgcgctcttggaggacgacacctaccgatggtggctgaggctccagcgggagaagaagcccaataacttgaatgacaccatcaaggagctgttcagagtggtgcctgggaatgtggaccctatgctggagaagaggtcggtgggctgccggcgctgcgccgttgtgggcaactcgggcaacctgagggagtcttcttatgggcctgagatagacagtcacgactttgtcctcaggatgaacaaggcgcccacggcagggtttgaagctgatgttgggaccaagaccacccaccatctggtgtaccctgagagcttccgggagctgggagataatgtcagcatgatcctggtgcccttcaagaccatcgacttggagtgggtggtgagcgccatcaccacgggcaccatttcccacacctacatcccggttcctgcaaagatcagagtgaaacaggataagatcctgatctaccacccagccttcatcaagtatgtctttgacaactggctgcaagggcacgggcgatacccatctaccggcatcctctcggtcatcttctcaatgcatgtctgcgatgaggtggacttgtacggcttcggggcagacagcaaagggaactggcaccactactgggagaacaacccatccgcgggggcttttcgcaagacgggggtgcacgatgcagactttgagtctaacgtgacggccaccttggcctccatcaataaaatccggatcttcaaggggagatgacgcagtgaagggctgaggatggacgcactgtcacacctctgcatttccagccccagcatcttgctggagccgttccatcccggagcttggaggggcagcctcaggtgtgtgcctgggcaccgctcacagcctcttgcacccagccgttggcagcatctactcagcaaggtcactaagctctgccagcgtggcagagcatgtcttggaacctgtcttgagtggggacaacgtccccccactgctgccctagagctggggagacgctgggaaaggttcaacctccacacactaaaatcattttggctcctggggcaagcttggggaatgaatgtggaagatgcctatattctgagagacaggacagtttcccaggaagatgggcagagacttgagtggcgattacctccagcacagagacgtgccaggcggtgttggcgctcggggcgagatgctgcccttctttgcacgaagcctggcctcttgcttggcgtgataaccctgtcatcttcccaaagctcatttatgagccaccagaggctcctaccccaaagattttcacagaaacttgaggccaggtgccgtggctcacacctgtaatctgaacactttgggaggccgaggcgggaggatcacttgagcccaggagttcaagaccagcctgggcaacatagtgagactcctgtctctacaaaaataaaagatttaaaaaaattagccaggcacggtggcacacacttgtagccccagctactagggaggctgaggagggaggatctcttgtgcctaggagttcgaggctgcagtgggctgtgatcacaccactgcactccagcctgggcaacagagtgagaccctgtctctgaaaaaaaaaaaaaaa

SEQ ID NO: 16 - protein, 340 aa, Homo sapiens
  human wild-type ST3 beta-galactoside alpha-2, 3-sialyltransferase 1, transcript variant 1 (ST3GalI, ST3O), sialyltransferase 4A (SIAT4A, SIATFL), CMP-N-acetylneuraminate- beta-galactosamide- alpha-2,3-sialyltransferase, Gal-NAc6S
MVTLRKRTLKVLTFLVLFIFLTSFFLNYSHTMVATTWFPKQMVLELSENLKRLIKHRPCTCTHCIGQRKLSAWFDERFNQTMQPLLTAQNALLEDDTYRWWLRLQREKKPNNLNDTIKELFRVVPGNVDPMLEKRSVGCRRCAVVGNSGNLRESSYGPEIDSHDFVLRMNKAPTAGFEADVGTKTTHHLVYPESFRELGDNVSMILVPFKTIDLEWVVSAITTGTISHTYIPVPAKIRVKQDKILIYHPAFIKYVFDNWLQGHGRYPSTGILSVIFSMHVCDEVDLYGFGADSKGNWHHYWENNPSAGAFRKTGVHDADFESNVTATLASINKIRIFKGR

SEQ ID NO: 17 - DNA, 1023 bp, Pan troglodytes
  chimpanzee wild-type ST3 beta-galactoside alpha-2,3-sialyltransferase 1 (ST3GalI), sialyltransferase 4A (SIAT4A), alpha 2,3-sialyltransferase ST3Gal I
atggtgaccctgcggaagaggaccctgaaagtgctcaccttcctcgtgctcttcatcttcctcacctccttcttcctgaactactcccacaccatggtggccaccacctggttccccaagcagatggtcctggagctctccgagaatctgaagagactgatcaagcacaggccttgcacctgcacccactgcattgggcagcgcaagctctcggcctggttcgatgagaggttcaaccagaccgtgcagccgctgctgaccgcccagaacgcgctcttggaggacgacacctaccgatggtggctgaggctccagcgggagaagaagcccaataacttgaatgacaccatcaaggagctgttcagagtggtgcctgggaatgtggaccccatgctggagaagaggtcggtgggctgccggcgctgcgccgttgtgggcaactcgggcaacctgagggagtcttcttatgggcctgagatagacagtcacgactttgtcctcaggatgaacaaggcgcccacggcagggtttgaagctgatgttgggaccaagaccacccaccatctggtgtaccctgagagcttccgggagctgggagataatgtcagcatgatcctggtgcccttcaagaccatcgacttggagtgggtggtgagcgccatcaccacgggcaccatttcccacacctacaccccggtccttgtgaagatcagagtgaaacaggataagatcctgatctaccacccagccttcatcaagtatgtctttgacaactggctgcaagggcacgggcgatacccatctaccggcatcctctcggtcatcttctcaatgcatgtctgcgatgaggtggacttgtacggcttcggggcagacagcaaagggaactggcaccactactgggagaacaacccatccgcgggggcttttcgcaagacgggggtgcacgatgcagactttgagtctaatgtgacggccaccttggccgccatcaataaaatccggatcttcaaggggagatga

SEQ ID NO: 18 - protein, 340 aa, Pan troglodytes
  chimpanzee wild-type ST3 beta-galactoside alpha-2,3-sialyltransferase 1 (ST3GalI), sialyltransferase 4A (SIAT4A), alpha 2,3-sialyltransferase ST3Gal I
MVTLRKRTLKVLTFLVLFIFLTSFFLNYSHTMVATTWFPKQMVLELSENLKRLIKHRPCTCTHCIGQRKLSAWFDERFNQTVQPLLTAQNALLEDDTYRWWLRLQREKKPNNLNDTIKELFRVVPGNVDPMLEKRSVGCRRCAVVGNSGNLRESSYGPEIDSHDFVLRMNKAPTAGFEADVGTKTTHHLVYPESFRELGDNVSMILVPFKTIDLEWVVSAITTGTISHTYTPVLVKIRVKQDKILIYHPAFIKYVFDNWLQGHGRYPSTGILSVIFSMHVCDEVDLYGFGADSKGNWHHYWENNPSAGAFRKTGVHDADFESNVTATLAAINKIRIFKGR

SEQ ID NO: 19 - DNA, 1217 bp, Sus scrofa
  pig wild-type Gal beta-1,3 GalNAc alpha-2,3 sialyltransferase (ST3GalI), sialyltransferase 4A (SIAT4A)
cttcttgggaggtgctcgtccgttaggcgtgggttcctgcatcccatccctggggtgcccctgccccgcgccccggccggggaggcagacatggcccccatgaggaagaagagcaccctcaagctgctcacgctcctggtcctcttcatcttcctcacctccttcttcctcaactactcgcacaccgtggtcaccaccgcctggttccccaagcagatggtcatcgagctctccgagaacttcaagaagctcatgaaatacccctacaggccctgcacctgcacccgctgcatcgaagagcagagggtctccgcctggttcgatgagcgattcaaccggtccatgcagccgctgctgacggccaagaacgcgcacctggaggaagacacttacaagtggtggctgaggctccagcgggagaagcagcccaataacttgaacgacaccatcagggagctgttccaggtggtgcctgggaacgtggaccccctgctggagaagaggctggtcagctgccggcgctgcgccgtcgtgggcaactcgggcaacctgaaggagtcctactatgggcctcagatagacagccacgacttcgtgctgaggatgaacaaggcccccacggaggggtttgaggccgacgtcgggagcaagaccacccaccatttcgtgtaccccgagagcttccgggagctggcgcaggaggtcagcatgatcctggtccccttcaagaccaccgacctggagtgggtgatcagcgccaccaccaccggcaggatctcccacacctacgttcctgtccccgccaagatcaaagtcaaaaaggagaagatcctgatttatcacccggccttcatcaagtacgtcttcgacaggtggctgcagggccacgggcgctacccgtccactggcatcctctccgtgatcttctccctgcacatctgtgacgaggtggacttgtatggctttggggcggacagcaaagggaactggcaccactactgggagaacaacccttcggcgggggctttccgaaagaccggggtgcacgacggggacttcgagtccaacgtgacaaccatcttggcttccatcaacaagatccggatcttcaagggcagatgacgccgcgcaggttaaggacagttgcagcagctcacctctcgacgtccagccccgggaacttggtggccagcctcaggggtctgcccaggtgcccc

SEQ ID NO: 20 - protein, 343 aa, Sus scrofa
  pig wild-type Gal beta-1,3 GalNAc alpha-2,3 sialyltransferase (ST3GalI), sialyltransferase 4A (SIAT4A)
MAPMRKKSTLKLLTLLVLFIFLTSFFLNYSHTVVTTAWFPKQMVIELSENFKKLMKYPYRPCTCTRCIEEQRVSAWFDERFNRSMQPLLTAKNAHLEEDTYKWWLRLQREKQPNNLNDTIRELFQVVPGNVDPLLEKRLVSCRRCAVVGNSGNLKESYYGPQIDSHDFVLRMNKAPTEGFEADVGSKTTHHFVYPESFRELAQEVSMILVPFKTTDLEWVISATTTGTISHTYVPVPAKIKVKKEKILIYHPAFIKYVFDRWLQGHGRYPSTGILSVIFSLHICDEVDLYGFGADSKGNWHHYWENNPSAGAFRKTGVHDGDFESNVTTILASINKIRIFKGR

SEQ ID NO: 21 - DNA, 4996 bp, Mus musculus
  mouse wild-type ST3 beta-galactoside alpha-2, 3-sialyltransferase 1 (ST3GalI), sialyltransferase 4A (SIAT4A), CMP-N-acetylneuraminate: [beta-galactosidase alpha-2,3] sialytransferase, AI467004, MGC90735, 5330418N22Rik
cacaaacctaggcgtgtctccaacaggctgaagtttgccctagctgcttctgagatctccctgcggactccctttcctgctgcctgctcctgtgtctctgagatttctgttcagctctaggaggggggtctcctctcttccaccctcccatcaacacagaggacccagcaaagggtggaggtggtgaacatgaggaggaagaccctcaagtacctcaccttcttcctgctcttcatcttcctcacttcctttgtcctgaattactccaacacgggagtacccagtgcctggttccccaagcagatgctcctggaactctcagagaacttccgcaggttcatcaagtcacagccgtgcacctgcagacactgcatcagccaggacaaggtatcatattggttcgaccagcgcttcaacaagactatgcagcccctgctgacagtccacaacgctctgatggaggaggacacataccggtggtggctgaggctccagcgggagaggaaacccaacaacctgagcgacaccgtcaaggaactgtttcgcctggtgcctggcaatgtggatcctatgttgaacaagaggctggtgggctgccgacgctgtgcagttgtaggaaactccggtaacctgaaggactcctcgtatgggcctgagatcgacagccatgactttgtgctgaggatgaacaaggcacccacagtgggttttgaggcagacgttgggagccggaccacccaccatctcgtgtatcctgagagcttccgggagctgggagagaatgtcaacatggtcctggtccccttcaagaccaccgacctgcagtgggtgatcagcgccaccaccacaggcaccatcactcacacctatgtccccgtgcccccgaagatcaaagtgaaacaagagaagatcctgatctaccacccagccttcatcaagtatgtctttgacaactggcttcagggccatgggcggtatccatcgactggcatcctctccatcatcttctccattcatatctgtgatgaggtggacttatatggttttggggcagacagcaaaggaaattggcaccattactgggagaacaacccatcagcgggtgccttccgaaagacgggggttcacgatggtgacttcgagtacaacatcacaactaccctggcagccatcaataaaatccgcatcttcaaggggagatgatgctgcgacccattaaggatggacacaacacatcacacctctcaatttgcagccccaacatctcccaggagccagtgtgttccagacgcttccggggctgaactcggggtgttcccaggctttctggcagccccttgcaccctcatgttgttatcatctacttagcaaagttgtccaactctcccaggatacagagccagtctcgggcctgtctacagtgggagcactttcccaccgctgtcccctagctggggaaaagcgagccaataccaggaaaggtctagcctccaaacaagttattttgtcttctgagttggattggagggaggggatgggagaaaataaatcctgaagattcttatgttcaaaagagtgaacatttcccggaagtcatttagaggctgccactgaactgtgggcctcatgctcaactcagactctggctgggaatgtttgggccagtcatctgcagagccacactgggctacaccagcagaggggcaagataccgttctcctttgcatgaagcctagcctgttatttgagatgacatccccattgtccctttcttcaataaggtcaccggtgatggctaggcaaaacttctaactccagaggtcttccagggaagcctggccatgtgtggctgacaggagtgaaaccctggtcacagttctgcgtgattttccctccgtagtgatgctgatggccagagcaggtctgactacttccacgtgcctttgaacttgagaaagaagtgcatgcattggctgataggaagcaaaacccccaattggagaaaggggacctccaccagcacctcgctttgctgcatagctcaaaatctcttttcaaatgctattccgcacctccacccagaatataccaggcatgggatgggtcctcacttctatttcctcagactgctgggttcaggaccactaacaaccagcatcctcccatgggtcctgaacactgagccaagtccccagcagccaccttcctggactcgtttggggccgcactggggacactctagaaccacaactgaaatgcatgtacctcttcttcttggttctgtctgctctcccctccttcctctatggaaatcctgattatttattgatttgttaattcagctgaccctgtgcacactcttccccatggctgatcacaatgtgttttgttgctttgcctggcaagtgggagcaggaacagaagggagagcatcctggatgtatgtgttgagacatcaggacttggggcttgctggcgggcagatcctgtggcgccgacaatcctttgtagaatccatgtcacctccctttctagtacaagtgacttgccctcttcctcactgcagcagggaggagagcgggcacttggaggtaactgtcacctccttacagatgttcaataagagaaacctctgtttctgttgaaaaaatagtccctaaagaccaccacaacttcatcaaggcagattatgagagagggatcttgagggttgggccatctccctctactcagattcccctctgcaagtaagagctcgggagcccctctcaactttgggctgttcacagacatcaccaaagagcagacactaagactgtcatttcctaggagtctgtcctgggagctttgggattgcttttgtttaattttcgatggctcagaattcgggcttttaatgagggaacgataggtttcaccttttttttttccattttttattaggtatttagctcatttacatttccaatgctataccaaaagtcccccatacccacccacccccactcccctaccctcccactccccctttttggccctgccgttcccctgtactggggcatataaagtttgcgtgtccaatgggaaaatcttcctgccataaaacccttggctgaaaaggctagactcctgtgaggtccttgctgtggagggccctcctcaagggtcccagtttgaggaactcagcagatgtttgtgggtcagcccgtcaatccctacatcaaatcaaaaatagtggacgttggcttaggggtgagagttcaggtgtagctgggttctgatgcatgagagcaagtacctgcagtgaaggtgtagccatttccagctgctcaagaagttcttctgctctccattggcccctcccactccctctgcccccacctgccttcctgccccctctccctccctccctcactccctccctccctccctccctccctccctccctccctccctccctcttccggccaaggagccaatggatctctttcctataaccctcatccctcccctgtgtttcactggcttaacggtgggctcttaactgaatagaatacagttggttgtggccagaattaagtgcaccaattagtttaaggtaactggagctttccctgggtgtgagtgactgagaacaggaaaggccttccccgaagagactgggcagactgctgacagaaaagctagtcctgggttccaggcagagagcaaagggctggcttccagtacagattcttagtagaagaatctactaagtccaagggaattggaggtgggggttctcatcatcaccccagatgactaaccaggaggctggtgtttcttccactcccctcagcctctgttaggagagagagaagccgatctgtagcctttggttcctgggcatcagacatcattcaataagccatgtgggcagggttagtggaacagacccccaaaacagtgatagaaccagcaaccgtctaggtctgtgacagtctaaaggtctggagaccatagaaggatagatacacagtggatattgagaacctagagggtggcttcacctgtgcccgtcaaagaagcccctgacatgcagtgtgggggaggccctagataaccctgcacagaggacagagggacagctgtatgccctcaggaactggcagttggaaccacaggttctatcatagccaggtggctatgaggtgggaaagattggatctaggcagctcagccacaaaggcctggctagccaattttcagatagcatcagctctcctctttgtcatcctctcctgtgccgcaggtgttacctaagacctctaaaaagcaccagagggtgaagaaagaaagcagggaccagcagagtagtgccccgagctttcagcctcaaagttagtaaatgcgtcatcagccaagcccggcctggaacctgcttccgcctgctgaaccccatcttgaccttcctaacctctctgctcatttagcttttaccctggctccgcctgcccagggtctgccccatggcaaccgctttaaccctttctccagagcctaatccggatcttgcacaccctaccagatgaacatgggtaactcctgctgccccccacctccttgcacaatcactttaaccatttcccaagagcctgatctggactttgctcaacccaccctagtaggtagacatgggttcctcctgcacctctaggcacaatcactttagccctttcccaagtctgcttccgagccaaagttttccaagcactttctgcccgaaagctcttccttccttgcctcctgtgacccagcattgcctgtttaaactttgctttctcctgcccacgtcttgtaatcctcctgattggtctgaatgcatttccaatgatgtcagggagtctgccttcctcagcctcggctccattttatttattgttgaatatttccaattattcaaatcaaagtgaataaaaaaagagctattttattgttaaaaaaaaaaaaaaaaaaa

SEQ ID NO: 22 - protein, 337 aa, Mus musculus
  mouse wild-type ST3 beta-galactoside alpha-2, 3-sialyltransferase 1 (ST3GalI), sialyltransferase 4A (SIAT4A), CMP-N-acetylneuraminate: [beta-galactosidase alpha-2,3] sialytransferase
MRRKTLKYLTFFLLFIFLTSFVLNYSNTGVPSAWFPKQMLLELSENFRRFIKSQPCTCRHCISQDKVSYWFDQRFNKTMQPLLTVHNALMEEDTYRWWLRLQRERKPNNLSDTVKELFRLVPGNVDPMLNKRLVGCRRCAVVGNSGNLKDSSYGPEIDSHDFVLRMNKAPTVGFEADVGSRTTHHLVYPESFRELGENVNMVLVPFKTTDLQWVISATTTGTITHTYVPVPPKIKVKQEKILIYHPAFIKYVFDNWLQGHGRYPSTGILSIIFSIHICDEVDLYGFGADSKGNWHHYWENNPSAGAFRKTGVHDGDFEYNITTTLAAINKIRIFKGR

SEQ ID NO: 23 - DNA, 1838 bp, Gallus gallus
  chicken wild-type ST3 beta-galactoside alpha-2,3-sialyltransferase 1 (ST3GalI)
cccatggacagaggtactgcaggcagtaggtgcagcctaggccccaggtggtgaccggccccaagaaggccggcagcagcctgtccttgtggccttagcttccccccacgtcgtgctccaccagcagcaagatggtcaccgtcaggaaaaggaacgtgaaggtcttcacattcgccttcgtactcatcacggtgacgtcattcctgctgaactacaagcaccaggtgaccatgaccacttgggatcctaaacatatcatcagtcagttttctgagcaagtccgaaaactcatcaaatttcctaggaggccgtgcagctgtagcacctgtatttccgagctgggacattccctctggttcgaccagaggtttaactcaactatgcaacctttcctgacctcacaaaatgccttgatcccagaggacagctacaggtggtggctgaaactgcaaggagagaaatctccaaagaatattaatgatactctcaaggaattgtttgggatcattcctggggacagggacccactgcaggagcgaggcactttctcatgcagacggtgtgccgtcgttggcaactccggcaaccttcgtcagtctcaatatggccaagatattgactcccatgactttgtgctcagaatgaaccgtgcacccaccattggctacgaatcagatgttgggagcaagactacccaccactttgtttatccagagagctacaaagagctggcagaaaatgtgagcatgatcgtgatccccttcaaaaccctggacctgcgctggattgttaccgctctcaccacaggcactatcaacttcacatatgttcctgttccacggaaaatcaaagtcagaaaagaaaaggtcctgatttacaatccatcctttatcaaatacgtctatgaaaactggcttcagaatcatggaagatacccttccacaggccttctttctgtgatatttgcactccatgtatgcgatgaggtgaatgtgtatggttttggagcagacagcaaaggacactggcatcactactgggaaaataatgcttcagctggggctttccgacagacaggtgtccatgatggagattttgagttcaatgtaactttgactcttgcctccattgaaaaaataaaatttttcaagggcagatgaccctagccacagggacaagcaggggctgcaatttccaacatgcagcagcacaaagctcagtgaagatgatctggatgacagccaggtttgaaggtgtgaatctggagtggattcggagtgtcacactgctgcagtgctcaccacagggagctcagctgaggaacagattcaatgctgcacttgatttgctatctatagatagctgggaactaccgatcagctgttggaataaatgacatcctgactcactccttagtctctacggattgattcctgaattactggtgaaagatctacctgttgcaattcagggaaacctagacacaggaactgctgtttgtatgtccattctttccttgagtaaggcaagaaatccttgaagaacatggaaaatgtcttctgggatttgatacctactagagtagctctgaacatcatagtaaggattacctcaaagaaattaattcagttctgctgttaatcttctttttgaattctcttctgtttcctctattacttctggtttcatgcactataaatcaaaaacgtgatgaagttgccggagtagtaactgtttctaccattgccacattctgcattgatggcatctagaaaatacagatcaattcacctgtgatcattacttatttattattgcttcctatacagccatagtaa

SEQ ID NO: 24 - protein, 342 aa, Gallus gallus
  chicken wild-type ST3 beta-galactoside alpha-2,3-sialyltransferase 1 (ST3GalI)
MVTVRKRNVKVFTFAFVLITVTSFLLNYKHQVTMTTWDPKHIISQFSEQVRKLIKFPRRPCSCSTCISELGHSLWFDQRFNSTMQPFLTSQNALIPEDSYRWWLKLQGEKSPKNINDTLKELFGIIPGDRDPLQERGTFSCRRCAVVGNSGNLRQSQYGQDIDSHDFVLRMNRAPTIGYESDVGSKTTHHFVYPESYKELAENVSMIVIPFKTLDLRWIVTALTTGTINFTYVPVPRKIKVRKEKVLIYNPSFIKYVYENWLQNHGRYPSTGLLSVIFALHVCDEVNVYGFGADSKGHWHHYWENNASAGAFRQTGVHDGDFEFNVTLTLASIEKIKFFKGR

SEQ ID NO: 25 - protein, 277 aa, Sus scrofa
  amino acid positions 65-341 of pig wild-type Gal beta-1,3 GalNAc alpha-2,3 sialyltransferase (ST3GalI), sialyltransferase 4A (SIAT4A)
TRCIEEQRVSAWFDERFNRSMQPLLTAKNAHLEEDTYKWWLRLQREKQPNNLNDTIRELFQVVPGNVDPLLEKRLVSCRRCAVVGNSGNLKESYYGPQIDSHDFVLRMNKAPTEGFEADVGSKTTHHFVYPESFRELAQEVSMILVPFKTTDLEWVISATTTGRISHTYVPVPAKIKVKKEKILIYHPAFIKYVFDRWLQGHGRYPSTGILSVIFSLHICDEVDLYGFGADSKGNWHHYWENNPSAGAFRKTGVHDGDFESNVTTILASINKIRIFK

SEQ ID NO: 26 - protein, 313 aa, Homo sapiens
  amino acid positions 277-589 of human wild-type ST3 beta-galactoside alpha-2,3-sialyltransferase 1 (ST3GalI), sialyltransferase 4A (SIAT4A, SIATFL), CMP-N-acetylneuraminate-beta-galactosamide-alpha-2, 3-sialyltransferase, Gal-NAc6S
TTCPDSVKIKASKSLWLQKLFLPNLTLFLDSRHFNQSEWDRLEHFAPPFGFMELNYSLVQKVVTRFPPVPQQQLLLASLPAGSLRCITCAVVGNGGILNNSHMGQEIDSHDYVFRLSGALIKGYEQDVGTRTSFYGFTAFSLTQSLLILGNRGFKNVPLGKDVRYLHFLEGTRDYEWLEALLMNQTVMSKNLFWFRHRPQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQ

SEQ ID NO: 27 - skipped

SEQ ID NO: 28 - protein, 316 aa, Sus scrofa
  amino acid positions 28-343 of pig wild-type Gal beta-1,3 GalNAc alpha-2,3 sialyltransferase (ST3GalI), sialyltransferase 4A (SIAT4A)
NYSHTVVTTAWFPKQMVIELSENFKKLMKYPYRPCTCTRCIEEQRVSAWFDERFNRSMQPLLTAKNAHLEEDTYKWWLRLQREKQPNNLNDTIRELFQVVPGNVDPLLEKRLVSCRRCAVVGNSGNLKESYYGPQIDSHDFVLRMNKAPTEGFEADVGSKTTHHFVYPESFRELAQEVSMILVPFKTTDLEWVISATTTGRISHTYVPVPAKIKVKKEKILIYHPAFIKYVFDRWLQGHGRYPSTGILSVIFSLHICDEVDLYGFGADSKGNWHHYWENNPSAGAFRKTGVHDGDFESNVTTILASINKIRIFKGR

SEQ ID NO: 29 - protein, 600 aa, Homo sapiens
  human GalNAc alpha-2,6-sialyltransferase I, alpha-N-acetylgalactosaminide alpha-2,6-sialyltransferase 1, sialyltransferase 7A (ST6GalNAcI, SIAT7A)
MRSCLWRCRHLSQGVQWSLLLAVLVFFLFALPSFIKEPQTKPSRHQRTENIKERSLQSLAKPKSQAPTRARRTTIYAEPVPENNALNTQTQPKAHTTGDRGKEANQAPPEEQDKVPHTAQRAAWKSPEKEKTMVNTLSPRGQDAGMASGRTEAQSWKSQDTKTTQGNGGQTRKLTASRTVSEKHQGKAATTAKTLIPKSQHRMLAPTGAVSTRTRQKGVTTAVIPPKEKKPQATPPPAPFQSPTTQRNQRLKAANFKSEPRWDFEEKYSFEIGGLQTTCPDSVKIKASKSLWLQKLFLPNLTLFLDSRHFNQSEWDRLEHFAPPFGFMELNYSLVQKVVTRFPPVPQQQLLLASLPAGSLRCITCAVVGNGGILNNSHMGQEIDSHDYVFRLSGALIKGYEQDVGTRTSFYGFTAFSLTQSLLILGNRGFKNVPLGKDVRYLHFLEGTRDYEWLEALLMNQTVMSKNLFWFRHRPQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 30 - protein, 16 aa, Artificial Sequence
  preferred sequence insert (“X” peptide) in preferredembodiments of internal constructs of ST3GalI/St6GalNAcI fusion and preferred embodiments of ST6GalNAcI N-linked glycosylation mutants
PFGFMELNYSLVQKVV

SEQ ID NO: 31 - protein, 16 aa, Artificial Sequence
  preferred sequence insert (“X” peptide) in preferredembodiments of internal constructs of ST3GalI/St6GalNAcI fusion and preferred embodiments of ST6GalNAcI N-linked glycosylation mutants
PFGFMELIYSLVQKVV

SEQ ID NO: 32 - protein, 16 aa, Artificial Sequence
  preferred sequence insert (“X” peptide) in preferred embodiments of internal constructs of ST3GalI/St6GalNAcI fusion and preferred embodiments of ST6GalNAcI N-linked glycosylation mutants
PNNLNDTIRELFQVVP

SEQ ID NO: 33 - protein, 16 aa, Artificial Sequence
  preferred sequence insert (“X” peptide) in preferredembodiments of internal constructs of ST3GalI/St6GalNAcI fusion and preferred embodiments of ST6GalNAcI N-linked glycosylation mutants
PFGFMETIRELVQKVV

SEQ ID NO: 34 - protein, 16 aa, Artificial Sequence
  preferred sequence insert (“X” peptide) in preferredembodiments of internal constructs of ST3GalI/St6GalNAcI fusion and preferred embodiments of ST6GalNAcI N-linked glycosylation mutants
PFGFMELNYELVQKVV

SEQ ID NO: 35 - protein, 16 aa, Artificial Sequence
  preferred sequence insert (“X” peptide) in preferredembodiments of internal constructs of ST3GalI/St6GalNAcI fusion and preferred embodiments of ST6GalNAcI N-linked glycosylation mutants
PFGFMELKYSLVQKVV

SEQ ID NO: 36 - protein, 12 aa, Artificial Sequence
  preferred sequence insert (“Z” peptide) in preferredembodiments of internal constructs of ST3GalI/St6GalNAcI fusion and preferred embodiments of ST6GalNAcI N-linked glycosylation mutants
GGILNNSHMGQE

SEQ ID NO: 37 - protein, 12 aa, Artificial Sequence
  preferred sequence insert (“Z” peptide) in preferredembodiments of internal constructs of ST3GalI/St6GalNAcI fusion and preferred embodiments of ST6GalNAcI N-linked glycosylation mutants
GGILKNSHMGQE

SEQ ID NO: 38 - protein, 12 aa, Artificial Sequence
  preferred sequence insert (“Z” peptide) in preferredembodiments of internal constructs of ST3GalI/St6GalNAcI fusion and preferred embodiments of ST6GalNAcI N-linked glycosylation mutants
SGNLKESYYGPQ

SEQ ID NO: 39 - protein, 12 aa, Artificial Sequence
  preferred sequence insert (“Z” peptide) in preferredembodiments of internal constructs of ST3GalI/St6GalNAcI fusion and preferred embodiments of ST6GalNAcI N-linked glycosylation mutants
GGILKESHMGQE

SEQ ID NO: 40 - protein, 21 aa, Artificial Sequence
  preferred sequence insert (“B” peptide) in preferredembodiments of internal constructs of ST3GalI/St6GalNAcI fusion and preferred embodiments of ST6GalNAcI N-linked glycosylation mutants
LEALMNQTVMSKNLFWFRHRP

SEQ ID NO: 41 - protein, 21 aa, Artificial Sequence
  preferred sequence insert (“Z” peptide) in preferredembodiments of internal constructs of ST3GalI/St6GalNAcI fusion and preferred embodiments of ST6GalNAcI N-linked glycosylation mutants
LEALISATTTGRISHTYVPVP

SEQ ID NO: 42 - protein, 21 aa, Artificial Sequence
  preferred sequence insert (“Z” peptide) in preferredembodiments of internal constructs of ST3GalI/St6GalNAcI fusion and preferred embodiments of ST6GalNAcI N-linked glycosylation mutants
LEALMSQTVMSKNLFWFRHRP

SEQ ID NO: 43 - protein, 21 aa, Artificial Sequence
  preferred sequence insert (“B” peptide) in preferredembodiments of internal constructs of ST3GalI/St6GalNAcI fusion and preferred embodiments of ST6GalNAcI N-linked glycosylation mutants
LEALMKQTVMSKNLFWFRHRP

SEQ ID NO: 44 - protein, 17 aa, Artificial Sequence
  preferred sequence insert (“B” peptide) in preferredembodiments of internal constructs of ST3GalI/St6GalNAcI fusion and preferred embodiments of ST6GalNAcI N-linked glycosylation mutants
MNQTVMSKNLFWFRHRP

SEQ ID NO: 45 - protein, 17 aa, Artificial Sequence
  preferred sequence insert (“B” peptide) in preferredembodiments of internal constructs of ST3GalI/St6GalNAcI fusion and preferred embodiments of ST6GalNAcI N-linked glycosylation mutants
ISATTTGRISHTYVPVP

SEQ ID NO: 46 - protein, 17 aa, Artificial Sequence
  preferred sequence insert (“B” peptide) in preferredembodiments of internal constructs of ST3GalI/St6GalNAcI fusion and preferred embodiments of ST6GalNAcI N-linked glycosylation mutants
MSQTVMSKNLFWFRHRP

SEQ ID NO: 47 - protein, 17 aa, Artificial Sequence
  preferred sequence insert (“B” peptide) in preferredembodiments of internal constructs of ST3GalI/St6GalNAcI fusion and preferred embodiments of ST6GalNAcI N-linked glycosylation mutants
MKQTVMSKNLFWFRHRP

SEQ ID NO: 48 - protein, 30 aa, Artificial Sequence
  preferred sequence insert (“J” peptide) in preferredembodiments of internal constructs of ST3GalI/St6GalNAcI fusion and preferred embodiments of ST6GalNAcI N-linked glycosylation mutants
LWLQKLFLPNLTLFLDSRHFNQSEWDRLEH

SEQ ID NO: 49 - protein, 30 aa, Artificial Sequence
  preferred sequence insert (“J” peptide) in preferredembodiments of internal constructs of ST3GalI/St6GalNAcI fusion and preferred embodiments of ST6GalNAcI N-linked glycosylation mutants
LWLQKLFLPNLTLFLDSRHFKQSEWDRLEH

SEQ ID NO: 50 - protein, 30 aa, Artificial Sequence
  preferred sequence insert (“J” peptide) in preferredembodiments of internal constructs of ST3GalI/St6GalNAcI fusion and preferred embodiments of ST6GalNAcI N-linked glycosylation mutants
LWLQKLFLPKLTLFLDSRHFNQSEWDRLEH

SEQ ID NO: 51 - protein, 30 aa, Artificial Sequence
  preferred sequence insert (“J” peptide) in preferredembodiments of internal constructs of ST3GalI/St6GalNAcI fusion and preferred embodiments of ST6GalNAcI N-linked glycosylation mutants
LWLQKLFLPKLTLFLDSRHFKQSEWDRLEH

SEQ ID NO: 52 - protein, 30 aa, Artificial Sequence
  preferred sequence insert (“J” peptide) in preferredembodiments of internal constructs of ST3GalI/St6GalNAcI fusion and preferred embodiments of ST6GalNAcI N-linked glycosylation mutants
LWFDERFNRSMQPLLTAKNAHLEEWDRLEH

SEQ ID NO: 53 - protein, 30 aa, Artificial Sequence
  preferred sequence insert (“J” peptide) in preferredembodiments of internal constructs of ST3GalI/St6GalNAcI fusion and preferred embodiments of ST6GalNAcI N-linked glycosylation mutants
LWLQKLFLPSLTLFLDSRHFHQSEWDRLEH

SEQ ID NO: 54 - protein, 30 aa, Artificial Sequence
  preferred sequence insert (“J” peptide) in preferredembodiments of internal constructs of ST3GalI/St6GalNAcI fusion and preferred embodiments of ST6GalNAcI N-linked glycosylation mutants
LWLQKLFLPSMQLFLDSRHFNQSEWDRLEH

SEQ ID NO: 55 - protein, 30 aa, Artificial Sequence
  preferred sequence insert (“J” peptide) in preferredembodiments of internal constructs of ST3GalI/St6GalNAcI fusion and preferred embodiments of ST6GalNAcI N-linked glycosylation mutants
LWLQKLFLPNLTLFLDSRHFHLEEWDRLEH

SEQ ID NO: 56 - protein, 30 aa, Artificial Sequence
  preferred sequence insert (“J” peptide) in preferredembodiments of internal constructs of ST3GalI/St6GalNAcI fusion and preferred embodiments of ST6GalNAcI N-linked glycosylation mutants
LWLQKLFLPSMGLFLDSRHFNQSEWDRLEH

SEQ ID NO: 57 - protein, 30 aa, Artificial Sequence
  preferred sequence insert (“J” peptide) in preferredembodiments of internal constructs of ST3GalI/St6GalNAcI fusion and preferred embodiments of ST6GalNAcI N-linked glycosylation mutants
LWLQKLFLPSLTLFLDSRHFNQSEWDRLEH

SEQ ID NO: 58 - protein, 30 aa, Artificial Sequence
  preferred sequence insert (“J” peptide) in preferredembodiments of internal constructs of ST3GalI/St6GalNAcI fusion and preferred embodiments of ST6GalNAcI N-linked glycosylation mutants
LWLQKLFLPNLTLFLDSRHFHQSEWDRLEH

SEQ ID NO: 59 - protein, 30 aa, Artificial Sequence
  preferred sequence insert (“J” peptide) in preferredembodiments of internal constructs of ST3GalI/St6GalNAcI fusion and preferred embodiments of ST6GalNAcI N-linked glycosylation mutants
LWLQKLFLPNLQLFLDSRHFNQSEWDRLEH

SEQ ID NO: 60 - protein, 30 aa, Artificial Sequence
  preferred sequence insert (“J” peptide) in preferredembodiments of internal constructs of ST3GalI/St6GalNAcI fusion and preferred embodiments of ST6GalNAcI N-linked glycosylation mutants
LWLQKLFLPNLTLFLDSRHFNQEEWDRLEH

SEQ ID NO: 61 - protein, 30 aa, Artificial Sequence
  preferred sequence insert (“J” peptide) in preferredembodiments of internal constructs of ST3GalI/St6GalNAcI fusion and preferred embodiments of ST6GalNAcI N-linked glycosylation mutants
LWLQKLFLPNLQLFLDSRHFNQEEWDRLEH

SEQ ID NO: 62 - protein, 35 aa, Artificial Sequence
  preferred sequence insert (“J” peptide) in preferredembodiments of internal constructs of ST3GalI/St6GalNAcI fusion and preferred embodiments of ST6GalNAcI N-linked glycosylation mutants
LWFDERFNRSMQPLLTAKNAHLEEDTYKWWDRLEH

SEQ ID NO: 63 - protein, 357 aa, Artificial Sequence
  preferred embodiment (Sequence 1) of amino terminal construct of ST3GalI/ST6GalNAcI fusion
NYSHTVVTTAWFPKQMVIELSENFKKLMKYPYRPCTCTRCIEEQRVSAWFDERFNRSMQPLLTAKNAHLEEWDRLEHFAPPFGFMELNYSLVQKVVTRFPPVPQQQLLLASLPAGSLRCITCAVVGNGGILNNSHMGQEIDSHDYVFRLSGALIKGYEQDVGTRTSFYGFTAFSLTQSLLILGNRGFKNVPLGKDVRYLHFLEGTRDYEWLEALLMNQTVMSKNLFWFRHRPQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 64 - protein, 362 aa, Artificial Sequence
  preferred embodiment (Sequence 2) of amino terminal construct of ST3GalI/ST6GalNAcI fusion
NYSHTVVTTAWFPKQMVIELSENFKKLMKYPYRPCTCTRCIEEQRVSAWFDERFNRSMQPLLTAKNAHLEEDTYKWWDRLEHFAPPFGFMELNYSLVQKVVTRFPPVPQQQLLLASLPAGSLRCITCAVVGNGGILNNSHMGQEIDSHDYVFRLSGALIKGYEQDVGTRTSFYGFTAFSLTQSLLILGNRGFKNVPLGKDVRYLHFLEGTRDYEWLEALLMNQTVMSKNLFWFRHRPQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 65 - protein, 362 aa, Artificial Sequence
  preferred embodiment (Sequence 3) of amino terminal construct of ST3GalI/ST6GalNAcI fusion
NYSHTVVTTAWFPKQMVIELSENFKKLMKYPYRPCTCTRCIEEQRVSAWFDERFNRSMQPLLTAKNAHLEEDTYKWWLRLQHFAPPFGFMELNYSLVQKVVTRFPPVPQQQLLLASLPAGSLRCITCAVVGNGGILNNSHMGQEIDSHDYVFRLSGALIKGYEQDVGTRTSFYGFTAFSLTQSLLILGNRGFKNVPLGKDVRYLHFLEGTRDYEWLEALLMNQTVMSKNLFWFRHRPQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 66 - protein, 357 aa, Artificial Sequence
  preferred embodiment (Sequence 3) of amino terminal construct of ST3GalI/ST6GalNAcI fusion
NYSHTVVTTAWFPKQMVIELSENFKKLMKYPYRPCTCTRCIEEQRVSAWFDERFNRSMQPLLTAKNAHLEEWLRLQHFAPPFGFMELNYSLVQKVVTRFPPVPQQQLLLASLPAGSLRCITCAVVGNGGILNNSHMGQEIDSHDYVFRLSGALIKGYEQDVGTRTSFYGFTAFSLTQSLLILGNRGFKNVPLGKDVRYLHFLEGTRDYEWLEALLMNQTVMSKNLFWFRHRPQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 67 - protein, 363 aa, Artificial Sequence
  preferred embodiment (Sequence 4) of amino terminal construct of ST3GalI/ST6GalNAcI fusion
NYSHTVVTTAWFPKQMVIELSENFKKLMKYPYRPCTCTRCIEEQRVSAWFDERFNRSMQPLLTAKNAHLEEDTYKWWLRLQREKQPNNLNDTIRELFQVVPGNVDPLLEKRLVLLASLPAGSLRCITCAVVGNGGILNNSHMGQEIDSHDYVFRLSGALIKGYEQDVGTRTSFYGFTAFSLTQSLLILGNRGFKNVPLGKDVRYLHFLEGTRDYEWLEALLMNQTVMSKNLFWFRHRPQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 68 - protein, 358 aa, Artificial Sequence
  preferred embodiment (Sequence 4) of amino terminal construct of ST3GalI/ST6GalNAcI fusion
NYSHTVVTTAWFPKQMVIELSENFKKLMKYPYRPCTCTRCIEEQRVSAWFDERFNRSMQPLLTAKNAHLEEWLRLQREKQPNNLNDTIRELFQVVPGNVDPLLEKRLVLLASLPAGSLRCITCAVVGNGGILNNSHMGQEIDSHDYVFRLSGALIKGYEQDVGTRTSFYGFTAFSLTQSLLILGNRGFKNVPLGKDVRYLHFLEGTRDYEWLEALLMNQTVMSKNLFWFRHRPQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 69 - protein, 353 aa, Artificial Sequence
  preferred embodiment (Sequence 4) of amino terminal construct of ST3GalI/ST6GalNAcI fusion
NYSHTVVTTAWFPKQMVIELSENFKKLMKYPYRPCTCTRCIEEQRVSAWFDERFNRSMQPLLTAKNAHLEEDTYKWWLRLQREKQPNNLNDTIRELFQVVPGNVDPLLEKRLVRCITCAVVGNGGILNNSHMGQEIDSHDYVFRLSGALIKGYEQDVGTRTSFYGFTAFSLTQSLLILGNRGFKNVPLGKDVRYLHFLEGTRDYEWLEALLMNQTVMSKNLFWFRHRPQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 70 - protein, 348 aa, Artificial Sequence
  preferred embodiment (Sequence 4) of amino terminal construct of ST3GalI/ST6GalNAcI fusion
NYSHTVVTTAWFPKQMVIELSENFKKLMKYPYRPCTCTRCIEEQRVSAWFDERFNRSMQPLLTAKNAHLEEWLRLQREKQPNNLNDTIRELFQVVPGNVDPLLEKRLVRCITCAVVGNGGILNNSHMGQEIDSHDYVFRLSGALIKGYEQDVGTRTSFYGFTAFSLTQSLLILGNRGFKNVPLGKDVRYLHFLEGTRDYEWLEALLMNQTVMSKNLFWFRHRPQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 71 - protein, 363 aa, Artificial Sequence
  preferred embodiment (Sequence 4) of amino terminal construct of ST3GalI/ST6GalNAcI fusion
  MOD_RES (66)...(113): Xaa = any amino acid
NYSHTVVTTAWFPKQMVIELSENFKKLMKYPYRPCTCTRCIEEQRVSAWFDERFNRSMQPLLTAKNAHLEEDTYKWWLRLQXXXXXXXXXXXXXXXXXXXXXXXXXXXXXXXXLLASLPAGSLRCITCAVVGNGGILNNSHMGQEIDSHDYVFRLSGALIKGYEQDVGTRTSFYGFTAFSLTQSLLILGNRGFKNVPLGKDVRYLHFLEGTRDYEWLEALLMNQTVMSKNLFWFRHRPQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 72 - protein, 358 aa, Artificial Sequence
  preferred embodiment (Sequence 4) of amino terminal construct of ST3GalI/ST6GalNAcI fusion
  MOD_RES (61)...(108): Xaa = any amino acid
NYSHTVVTTAWFPKQMVIELSENFKKLMKYPYRPCTCTRCIEEQRVSAWFDERFNRSMQPLLTAKNAHLEEWLRLQXXXXXXXXXXXXXXXXXXXXXXXXXXXXXXXXLLASLPAGSLRCITCAVVGNGGILNNSHMGQEIDSHDYVFRLSGALIKGYEQDVGTRTSFYGFTAFSLTQSLLILGNRGFKNVPLGKDVRYLHFLEGTRDYEWLEALLMNQTVMSKNLFWFRHRPQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 73 - protein, 353 aa, Artificial Sequence
  preferred embodiment (Sequence 4) of amino terminal construct of ST3GalI/ST6GalNAcI fusion
  MOD_RES (66)...(113): Xaa = any amino acid
NYSHTVVTTAWFPKQMVIELSENFKKLMKYPYRPCTCTRCIEEQRVSAWFDERFNRSMQPLLTAKNAHLEEDTYKWWLRLQXXXXXXXXXXXXXXXXXXXXXXXXXXXXXXXXRCITCAVVGNGGILNNSHMGQEIDSHDYVFRLSGALIKGYEQDVGTRTSFYGFTAFSLTQSLLILGNRGFKNVPLGKDVRYLHFLEGTRDYEWLEALLMNQTVMSKNLFWFRHRPQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 74 - protein, 348 aa, Artificial Sequence
  preferred embodiment (Sequence 4) of amino terminal construct of ST3GalI/ST6GalNAcI fusion
  MOD_RES (61)...(108): Xaa = any amino acid
NYSHTVVTTAWFPKQMVIELSENFKKLMKYPYRPCTCTRCIEEQRVSAWFDERFNRSMQPLLTAKNAHLEEWLRLQXXXXXXXXXXXXXXXXXXXXXXXXXXXXXXXXRCITCAVVGNGGILNNSHMGQEIDSHDYVFRLSGALIKGYEQDVGTRTSFYGFTAFSLTQSLLILGNRGFKNVPLGKDVRYLHFLEGTRDYEWLEALLMNQTVMSKNLFWFRHRPQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 75 - protein, 361 aa, Artificial Sequence
  preferred embodiment (Sequence 5) of amino terminal construct of ST3GalI/ST6GalNAcI fusion
NYSHTVVTTAWFPKQMVIELSENFKKLMKYPYRPCTCTTCPDSVKIKASKSLWLQKLFLPNLTLFLDSRHFNQSEWDRLEHFAPPFGFMELNYSLVQKVVTRFPPVPQQQLLLASLPAGSLRCITCAVVGNGGILNNSHMGQEIDSHDYVFRLSGALIKGYEQDVGTRTSFYGFTAFSLTQSLLILGNRGFKNVPLGKDVRYLHFLEGTRDYEWLEALLMNQTVMSKNLFWFRHRPQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 76 - protein, 357 aa, Artificial Sequence
  preferred embodiment (Sequence 5) of amino terminal construct of ST3GalI/ST6GalNAcI fusion
NYSHTVVTTAWFPKQMVIELSENFKKLMKYPYRPCTCTTCPDSVKIKAWLQKLFLPNLTLFLDSRHFNQSEWDRLEHFAPPFGFMELNYSLVQKVVTRFPPVPQQQLLLASLPAGSLRCITCAVVGNGGILNNSHMGQEIDSHDYVFRLSGALIKGYEQDVGTRTSFYGFTAFSLTQSLLILGNRGFKNVPLGKDVRYLHFLEGTRDYEWLEALLMNQTVMSKNLFWFRHRPQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 77 - protein, 356 aa, Artificial Sequence
  preferred embodiment (Sequence 1) of internal construct of ST3GalI/ST6GalNAcI fusion
  MOD_RES (81)...(96): Xaa = any amino acid or positions 81-96 = one of SEQ ID NOS:30-35 (“X” peptide)
  MOD_RES (128)...(139): Xaa = any amino acid or positions 128-139 = one of SEQ ID NOS:36-39 (“Z” peptide)
  MOD_RES (211)...(231): Xaa = any amino acid or positions 211-231 = one of SEQ ID NOS:40-43 (“B” peptide)
NYSHTVVTTAWFPKQMVIELSENFKKLMKYPYRPCTCTRCIEEQRVSAWFDERFNRSMQPLLTAKNAHLEEWDRLEHFAPXXXXXXXXXXXXXXXXTRFPPVPQQQLLLASLPAGSLRCITCAVVGNXXXXXXXXXXXXIDSHDYVFRLSGALIKGYEQDVGTRTSFYGFTAFSLTQSLLILGNRGFKNVPLGKDVRYLHFLEGTRDYEWXXXXXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 78 - protein, 352 aa, Artificial Sequence
  preferred embodiment (Sequence 1) of internal construct of ST3GalI/ST6GalNAcI fusion
  MOD_RES (81)...(96): Xaa = any amino acid or positions 81-96 = one of SEQ ID NOS:30-35 (“X” peptide)
  MOD_RES (128)...(139): Xaa = any amino acid or positions 128-139 = one of SEQ ID NOS:36-39 (“Z” peptide)
  MOD_RES (211)...(227): Xaa = any amino acid or positions 211-227 = one of SEQ ID NOS:44-47 (“B” peptide)
NYSHTVVTTAWFPKQMVIELSENFKKLMKYPYRPCTCTRCIEEQRVSAWFDERFNRSMQPLLTAKNAHLEEWDRLEHFAPXXXXXXXXXXXXXXXXTRFPPVPQQQLLLASLPAGSLRCITCAVVGNXXXXXXXXXXXXIDSHDYVFRLSGALIKGYEQDVGTRTSFYGFTAFSLTQSLLILGNRGFKNVPLGKDVRYLHFLEGTRDYEWXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 79 - protein, 361 aa, Artificial Sequence
  preferred embodiment (Sequence 2) of internal construct of ST3GalI/ST6GalNAcI fusion
  MOD_RES (86)...(101): Xaa = any amino acid or positions 86-101 = one of SEQ ID NOS:30-35 (“X” peptide)
  MOD_RES (133)...(144): Xaa = any amino acid or positions 133-144 = one of SEQ ID NOS:36-39 (“Z” peptide)
  MOD_RES (216)...(236): Xaa = any amino acid or positions 216-236 = one of SEQ ID NOS:40-43 (“B” peptide)
NYSHTVVTTAWFPKQMVIELSENFKKLMKYPYRPCTCTRCIEEQRVSAWFDERFNRSMQPLLTAKNAHLEEDTYKWWDRLEHFAPXXXXXXXXXXXXXXXXTRFPPVPQQQLLLASLPAGSLRCITCAVVGNXXXXXXXXXXXXIDSHDYVFRLSGALIKGYEQDVGTRTSFYGFTAFSLTQSLLILGNRGFKNVPLGKDVRYLHFLEGTRDYEWXXXXXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 80 - protein, 357 aa, Artificial Sequence
  preferred embodiment (Sequence 2) of internal construct of ST3GalI/ST6GalNAcI fusion
  MOD_RES (86)...(101): Xaa = any amino acid or positions 86-101 = one of SEQ ID NOS:30-35 (“X” peptide)
  MOD_RES (133)...(144): Xaa = any amino acid or positions 133-144 = one of SEQ ID NOS:36-39 (“Z” peptide)
  MOD_RES (216)...(232): Xaa = any amino acid or positions 216-232 = one of SEQ ID NOS:44-47 (“B” peptide)
NYSHTVVTTAWFPKQMVIELSENFKKLMKYPYRPCTCTRCIEEQRVSAWFDERFNRSMQPLLTAKNAHLEEDTYKWWDRLEHFAPXXXXXXXXXXXXXXXXTRFPPVPQQQLLLASLPAGSLRCITCAVVGNXXXXXXXXXXXXIDSHDYVFRLSGALIKGYEQDVGTRTSFYGFTAFSLTQSLLILGNRGFKNVPLGKDVRYLHFLEGTRDYEWXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 81 - protein, 361 aa, Artificial Sequence
  preferred embodiment (Sequence 3) of internal construct of ST3GalI/ST6GalNAcI fusion
  MOD_RES (86)...(101): Xaa = any amino acid or positions 86-101 = one of SEQ ID NOS:30-35 (“X” peptide)
  MOD_RES (133)...(144): Xaa = any amino acid or positions 133-144 = one of SEQ ID NOS:36-39 (“Z” peptide)
  MOD_RES (216)...(236): Xaa = any amino acid or positions 216-236 = one of SEQ ID NOS:40-43 (“B” peptide)
NYSHTVVTTAWFPKQMVIELSENFKKLMKYPYRPCTCTRCIEEQRVSAWFDERFNRSMQPLLTAKNAHLEEDTYKWWLRLQHFAPXXXXXXXXXXXXXXXXTRFPPVPQQQLLLASLPAGSLRCITCAVVGNXXXXXXXXXXXXIDSHDYVFRLSGALIKGYEQDVGTRTSFYGFTAFSLTQSLLILGNRGFKNVPLGKDVRYLHFLEGTRDYEWXXXXXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 82 - protein, 356 aa, Artificial Sequence
  preferred embodiment (Sequence 3) of internal construct of ST3GalI/ST6GalNAcI fusion
  MOD_RES (81)...(96): Xaa = any amino acid or positions 81-96 = one of SEQ ID NOS:30-35 (“X” peptide)
  MOD_RES (128)...(139): Xaa = any amino acid or positions 128-139 = one of SEQ ID NOS:36-39 (“Z” peptide)
  MOD_RES (211)...(231): Xaa = any amino acid or positions 211-231 = one of SEQ ID NOS:40-43 (“B” peptide)
NYSHTVVTTAWFPKQMVIELSENFKKLMKYPYRPCTCTRCIEEQRVSAWFDERFNRSMQPLLTAKNAHLEEWLRLQHFAPXXXXXXXXXXXXXXXXTRFPPVPQQQLLLASLPAGSLRCITCAVVGNXXXXXXXXXXXXIDSHDYVFRLSGALIKGYEQDVGTRTSFYGFTAFSLTQSLLILGNRGFKNVPLGKDVRYLHFLEGTRDYEWXXXXXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 83 - protein, 357 aa, Artificial Sequence
  preferred embodiment (Sequence 3) of internal construct of ST3GalI/ST6GalNAcI fusion
  MOD_RES (86)...(101): Xaa = any amino acid or positions 86-101 = one of SEQ ID NOS:30-35 (“X” peptide)
  MOD_RES (133)...(144): Xaa = any amino acid or positions 133-144 = one of SEQ ID NOS:36-39 (“Z” peptide)
  MOD_RES (216)...(232): Xaa = any amino acid or positions 216-232 = one of SEQ ID NOS:30-35 (“B” peptide)
NYSHTVVTTAWFPKQMVIELSENFKKLMKYPYRPCTCTRCIEEQRVSAWFDERFNRSMQPLLTAKNAHLEEDTYKWWLRLQHFAPXXXXXXXXXXXXXXXXTRFPPVPQQQLLLASLPAGSLRCITCAVVGNXXXXXXXXXXXXIDSHDYVFRLSGALIKGYEQDVGTRTSFYGFTAFSLTQSLLILGNRGFKNVPLGKDVRYLHFLEGTRDYEWXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 84 - protein, 352 aa, Artificial Sequence
  preferred embodiment (Sequence 3) of internal construct of ST3GalI/ST6GalNAcI fusion
  MOD_RES (81)...(96): Xaa = any amino acid or positions 81-96 = one of SEQ ID NOS:30-35 (“X” peptide)
  MOD_RES (128)...(139): Xaa = any amino acid or positions 128-139 = one of SEQ ID NOS:36-39 (“Z” peptide)
  MOD_RES (211)...(227): Xaa = any amino acid or positions 211-227 = one of SEQ ID NOS:44-47 (“B” peptide)
NYSHTVVTTAWFPKQMVIELSENFKKLMKYPYRPCTCTRCIEEQRVSAWFDERFNRSMQPLLTAKNAHLEEWLRLQHFAPXXXXXXXXXXXXXXXXTRFPPVPQQQLLLASLPAGSLRCITCAVVGNXXXXXXXXXXXXIDSHDYVFRLSGALIKGYEQDVGTRTSFYGFTAFSLTQSLLILGNRGFKNVPLGKDVRYLHFLEGTRDYEWXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 85 - protein, 359 aa, Artificial Sequence
  preferred embodiment (Sequence 4) of internal construct of ST3GalI/ST6GalNAcI fusion
  MOD_RES (134)...(145): Xaa = any amino acid or positions 134-145 = one of SEQ ID NOS:36-39 (“Z” peptide)
  MOD_RES (214)...(234): Xaa = any amino acid or positions 214-234 = one of SEQ ID NOS:40-43 (“B” peptide)
NYSHTVVTTAWFPKQMVIELSENFKKLMKYPYRPCTCTRCIEEQRVSAWFDERFNRSMQPLLTAKNAHLEEDTYKWWLRLQREKQPNNLNDTIRELFQVVPGNVDPLLEKRLVLLASLPAGSLRCITCAVVGNXXXXXXXXXXXXIDSHDYVFRLSGALIKGYEQDVGTRTSFYGFTAFSLTQSLLILGNRGFKNVPLGKDVRYLHFLEGTRDXXXXXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 86 - protein, 354 aa, Artificial Sequence
  preferred embodiment (Sequence 4) of internal construct of ST3GalI/ST6GalNAcI fusion
  MOD_RES (129)...(140): Xaa = any amino acid or positions 129-140 = one of SEQ ID NOS:30-35 (“Z” peptide)
  MOD_RES (209)...(229): Xaa = any amino acid or positions 209-229 = one of SEQ ID NOS:40-43 (“B” peptide)
NYSHTVVTTAWFPKQMVIELSENFKKLMKYPYRPCTCTRCIEEQRVSAWFDERFNRSMQPLLTAKNAHLEEWLRLQREKQPNNLNDTIRELFQVVPGNVDPLLEKRLVLLASLPAGSLRCITCAVVGNXXXXXXXXXXXXIDSHDYVFRLSGALIKGYEQDVGTRTSFYGFTAFSLTQSLLILGNRGFKNVPLGKDVRYLHFLEGTRDXXXXXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 87 - protein, 349 aa, Artificial Sequence
  preferred embodiment (Sequence 4) of internal construct of ST3GalI/ST6GalNAcI fusion
  MOD_RES (124)...(135): Xaa = any amino acid or positions 124-135 = one of SEQ ID NOS:36-39 (“Z” peptide)
  MOD_RES (204)...(224): Xaa = any amino acid or positions 204-224 = one of SEQ ID NOS:40-43 (“B” peptide)
NYSHTVVTTAWFPKQMVIELSENFKKLMKYPYRPCTCTRCIEEQRVSAWFDERFNRSMQPLLTAKNAHLEEDTYKWWLRLQREKQPNNLNDTIRELFQVVPGNVDPLLEKRLVRCITCAVVGNXXXXXXXXXXXXIDSHDYVFRLSGALIKGYEQDVGTRTSFYGFTAFSLTQSLLILGNRGFKNVPLGKDVRYLHFLEGTRDXXXXXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 88 - protein, 344 aa, Artificial Sequence
  preferred embodiment (Sequence 4) of internal construct of ST3GalI/ST6GalNAcI fusion
  MOD_RES (119)...(130): Xaa = any amino acid or positions 119-130 = one of SEQ ID NOS:36-39 (“Z” peptide)
  MOD_RES (199)...(219): Xaa = any amino acid or positions 199-219 = one of SEQ ID NOS:40-43 (“B” peptide)
NYSHTVVTTAWFPKQMVIELSENFKKLMKYPYRPCTCTRCIEEQRVSAWFDERFNRSMQPLLTAKNAHLEEWLRLQREKQPNNLNDTIRELFQVVPGNVDPLLEKRLVRCITCAVVGNXXXXXXXXXXXXIDSHDYVFRLSGALIKGYEQDVGTRTSFYGFTAFSLTQSLLILGNRGFKNVPLGKDVRYLHFLEGTRDXXXXXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 89 - protein, 359 aa, Artificial Sequence
  preferred embodiment (Sequence 4) of internal construct of ST3GalI/ST6GalNAcI fusion
  MOD_RES (82)...(113): Xaa = any amino acid or positions 82-113 = one of SEQ ID NOS:30-35 (“X” peptide)
  MOD_RES (134)...(145): Xaa = any amino acid or positions 134-145 = one of SEQ ID NOS:36-39 (“Z” peptide)
  MOD_RES (214)...(234): Xaa = any amino acid or positions 214-234 = one of SEQ ID NOS:40-43 (“B” peptide)
NYSHTVVTTAWFPKQMVIELSENFKKLMKYPYRPCTCTRCIEEQRVSAWFDERFNRSMQPLLTAKNAHLEEDTYKWWLRLQXXXXXXXXXXXXXXXXXXXXXXXXXXXXXXXXLLASLPAGSLRCITCAVVGNXXXXXXXXXXXXIDSHDYVFRLSGALIKGYEQDVGTRTSFYGFTAFSLTQSLLILGNRGFKNVPLGKDVRYLHFLEGTRDXXXXXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 90 - protein, 354 aa, Artificial Sequence
  preferred embodiment (Sequence 4) of internal construct of ST3GalI/ST6GalNAcI fusion
  MOD_RES (77)...(108): Xaa = any amino acid or positions 77-108 = one of SEQ ID NOS:30-35 (“X” peptide)
  MOD_RES (129)...(140): Xaa = any amino acid or positions 129-140 = one of SEQ ID NOS:36-39 (“Z” peptide)
  MOD_RES (209)...(229): Xaa = any amino acid or positions 209-229 = one of SEQ ID NOS:40-43 (“B” peptide)
NYSHTVVTTAWFPKQMVIELSENFKKLMKYPYRPCTCTRCIEEQRVSAWFDERFNRSMQPLLTAKNAHLEEWLRLQXXXXXXXXXXXXXXXXXXXXXXXXXXXXXXXXLLASLPAGSLRCITCAVVGNXXXXXXXXXXXXIDSHDYVFRLSGALIKGYEQDVGTRTSFYGFTAFSLTQSLLILGNRGFKNVPLGKDVRYLHFLEGTRDXXXXXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 91 - protein, 349 aa, Artificial Sequence
  preferred embodiment (Sequence 4) of internal construct of ST3GalI/ST6GalNAcI fusion
  MOD_RES (82)...(113): Xaa = any amino acid or positions 82-113 = one of SEQ ID NOS:30-35 (“X” peptide)
  MOD_RES (124)...(135): Xaa = any amino acid or positions 124-135 = one of SEQ ID NOS:36-39 (“Z” peptide)
  MOD_RES (204)...(224): Xaa = any amino acid or positions 204-224 = one of SEQ ID NOS:40-43 (“B” peptide)
NYSHTVVTTAWFPKQMVIELSENFKKLMKYPYRPCTCTRCIEEQRVSAWFDERFNRSMQPLLTAKNAHLEEDTYKWWLRLQXXXXXXXXXXXXXXXXXXXXXXXXXXXXXXXXRCITCAVVGNXXXXXXXXXXXXIDSHDYVFRLSGALIKGYEQDVGTRTSFYGFTAFSLTQSLLILGNRGFKNVPLGKDVRYLHFLEGTRDXXXXXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 92 - protein, 344 aa, Artificial Sequence
  preferred embodiment (Sequence 4) of internal construct of ST3GalI/ST6GalNAcI fusion
  MOD_RES (77)...(108): Xaa = any amino acid or positions 77-108 = one of SEQ ID NOS:30-35 (“X” peptide)
  MOD_RES (119)...(130): Xaa = any amino acid or positions 119-130 = one of SEQ ID NOS:36-39 (“Z” peptide)
  MOD_RES (199)...(219): Xaa = any amino acid or positions 199-219 = one of SEQ ID NOS:40-43 (“B” peptide)
NYSHTVVTTAWFPKQMVIELSENFKKLMKYPYRPCTCTRCIEEQRVSAWFDERFNRSMQPLLTAKNAHLEEWLRLQXXXXXXXXXXXXXXXXXXXXXXXXXXXXXXXXRCITCAVVGNXXXXXXXXXXXXIDSHDYVFRLSGALIKGYEQDVGTRTSFYGFTAFSLTQSLLILGNRGFKNVPLGKDVRYLHFLEGTRDXXXXXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 93 - protein, 355 aa, Artificial Sequence
  preferred embodiment (Sequence 4) of internal construct of ST3GalI/ST6GalNAcI fusion
  MOD_RES (134)...(145): Xaa = any amino acid or positions 134-145 = one of SEQ ID NOS:36-39 (“Z” peptide)
  MOD_RES (214)...(230): Xaa = any amino acid or positions 214-230 = one of SEQ ID NOS:44-47 (“B” peptide)
NYSHTVVTTAWFPKQMVIELSENFKKLMKYPYRPCTCTRCIEEQRVSAWFDERFNRSMQPLLTAKNAHLEEDTYKWWLRLQREKQPNNLNDTIRELFQVVPGNVDPLLEKRLVLLASLPAGSLRCITCAVVGNXXXXXXXXXXXXIDSHDYVFRLSGALIKGYEQDVGTRTSFYGFTAFSLTQSLLILGNRGFKNVPLGKDVRYLHFLEGTRDXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 94 - protein, 350 aa, Artificial Sequence
  preferred embodiment (Sequence 4) of internal construct of ST3GalI/ST6GalNAcI fusion
  MOD_RES (129)...(140): Xaa = any amino acid or positions 129-140 = one of SEQ ID NOS:36-39 (“Z” peptide)
  MOD_RES (209)...(225): Xaa = any amino acid or positions 209-225 = one of SEQ ID NOS:44-47 (“B” peptide)
NYSHTVVTTAWFPKQMVIELSENFKKLMKYPYRPCTCTRCIEEQRVSAWFDERFNRSMQPLLTAKNAHLEEWLRLQREKQPNNLNDTIRELFQVVPGNVDPLLEKRLVLLASLPAGSLRCITCAVVGNXXXXXXXXXXXXIDSHDYVFRLSGALIKGYEQDVGTRTSFYGFTAFSLTQSLLILGNRGFKNVPLGKDVRYLHFLEGTRDXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 95 - protein, 345 aa, Artificial Sequence
  preferred embodiment (Sequence 4) of internal construct of ST3GalI/ST6GalNAcI fusion
  MOD_RES (124)...(135): Xaa = any amino acid or positions 124-135 = one of SEQ ID NOS:36-39 (“Z” peptide)
  MOD_RES (204)...(220): Xaa = any amino acid or positions 204-220 = one of SEQ ID NOS:44-47 (“B” peptide)
NYSHTVVTTAWFPKQMVIELSENFKKLMKYPYRPCTCTRCIEEQRVSAWFDERFNRSMQPLLTAKNAHLEEDTYKWWLRLQREKQPNNLNDTIRELFQVVPGNVDPLLEKRLVRCITCAVVGNXXXXXXXXXXXXIDSHDYVFRLSGALIKGYEQDVGTRTSFYGFTAFSLTQSLLILGNRGFKNVPLGKDVRYLHFLEGTRDXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 96 - protein, 340 aa, Artificial Sequence
  preferred embodiment (Sequence 4) of internal construct of ST3GalI/ST6GalNAcI fusion
  MOD_RES (119)...(130): Xaa = any amino acid or positions 119-130 = one of SEQ ID NOS:36-39 (“Z” peptide)
  MOD_RES (199)...(215): Xaa = any amino acid or positions 199-215 = one of SEQ ID NOS:44-47 (“B” peptide)
NYSHTVVTTAWFPKQMVIELSENFKKLMKYPYRPCTCTRCIEEQRVSAWFDERFNRSMQPLLTAKNAHLEEWLRLQREKQPNNLNDTIRELFQVVPGNVDPLLEKRLVRCITCAVVGNXXXXXXXXXXXXIDSHDYVFRLSGALIKGYEQDVGTRTSFYGFTAFSLTQSLLILGNRGFKNVPLGKDVRYLHFLEGTRDXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 97 - protein, 355 aa, Artificial Sequence
  preferred embodiment (Sequence 4) of internal construct of ST3GalI/ST6GalNAcI fusion
  MOD_RES (82)...(113): Xaa = any amino acid or positions 82-113 = one of SEQ ID NOS:30-35 (“X” peptide)
  MOD_RES (134)...(145): Xaa = any amino acid or positions 134-145 = one of SEQ ID NOS:36-39 (“Z” peptide)
  MOD_RES (214)...(230): Xaa = any amino acid or positions 214-230 = one of SEQ ID NOS:44-47 (“B” peptide)
NYSHTVVTTAWFPKQMVIELSENFKKLMKYPYRPCTCTRCIEEQRVSAWFDERFNRSMQPLLTAKNAHLEEDTYKWWLRLQXXXXXXXXXXXXXXXXXXXXXXXXXXXXXXXXLLASLPAGSLRCITCAVVGNXXXXXXXXXXXXIDSHDYVFRLSGALIKGYEQDVGTRTSFYGFTAFSLTQSLLILGNRGFKNVPLGKDVRYLHFLEGTRDXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 98 - protein, 350 aa, Artificial Sequence
  preferred embodiment (Sequence 4) of internal construct of ST3GalI/ST6GalNAcI fusion
  MOD_RES (77)...(108): Xaa = any amino acid or positions 77-108 = one of SEQ ID NOS:30-35 (“X” peptide)
  MOD_RES (129)...(140): Xaa = any amino acid or positions 129-140 = one of SEQ ID NOS:36-39 (“Z” peptide)
  MOD_RES (209)...(225): Xaa = any amino acid or positions 209-225 = one of SEQ ID NOS:44-47 (“B” peptide)
NYSHTVVTTAWFPKQMVIELSENFKKLMKYPYRPCTCTRCIEEQRVSAWFDERFNRSMQPLLTAKNAHLEEWLRLQXXXXXXXXXXXXXXXXXXXXXXXXXXXXXXXXLLASLPAGSLRCITCAVVGNXXXXXXXXXXXXIDSHDYVFRLSGALIKGYEQDVGTRTSFYGFTAFSLTQSLLILGNRGFKNVPLGKDVRYLHFLEGTRDXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 99 - protein, 345 aa, Artificial Sequence
  preferred embodiment (Sequence 4) of internal construct of ST3GalI/ST6GalNAcI fusion
  MOD_RES (82)...(113): Xaa = any amino acid or positions 82-113 = one of SEQ ID NOS:30-35 (“X” peptide)
  MOD_RES (124)...(135): Xaa = any amino acid or positions 124-135 = one of SEQ ID NOS:36-39 (“Z” peptide)
  MOD_RES (204)...(220): Xaa = any amino acid or positions 204-220 = one of SEQ ID NOS:44-47 (“B” peptide)
NYSHTVVTTAWFPKQMVIELSENFKKLMKYPYRPCTCTRCIEEQRVSAWFDERFNRSMQPLLTAKNAHLEEDTYKWWLRLQXXXXXXXXXXXXXXXXXXXXXXXXXXXXXXXXRCITCAVVGNXXXXXXXXXXXXIDSHDYVFRLSGALIKGYEQDVGTRTSFYGFTAFSLTQSLLILGNRGFKNVPLGKDVRYLHFLEGTRDXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 100 - protein, 340 aa, Artificial Sequence
  preferred embodiment (Sequence 4) of internal construct of ST3GalI/ST6GalNAcI fusion
  MOD_RES (77)...(108): Xaa = any amino acid or positions 77-108 = one of SEQ ID NOS:30-35 (“X” peptide)
  MOD_RES (119)...(130): Xaa = any amino acid or positions 119-130 = one of SEQ ID NOS:36-39 (“Z” peptide)
  MOD_RES (199)...(215): Xaa = any amino acid or positions 199-215 = one of SEQ ID NOS:44-47 (“B” peptide)
NYSHTVVTTAWFPKQMVIELSENFKKLMKYPYRPCTCTRCIEEQRVSAWFDERFNRSMQPLLTAKNAHLEEWLRLQXXXXXXXXXXXXXXXXXXXXXXXXXXXXXXXXRCITCAVVGNXXXXXXXXXXXXIDSHDYVFRLSGALIKGYEQDVGTRTSFYGFTAFSLTQSLLILGNRGFKNVPLGKDVRYLHFLEGTRDXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 101 - protein, 360 aa, Artificial Sequence
  preferred embodiment (Sequence 5) of internal construct of ST3GalI/ST6GalNAcI fusion
  MOD_RES (52)...(81): Xaa = any amino acid or positions 52-81 = one of SEQ ID NOS:48-61 (“J” peptide)
  MOD_RES (85)...(100): Xaa = any amino acid or positions 85-100 = one of SEQ ID NOS:30-35 (“X” peptide)
  MOD_RES (132)...(143): Xaa = any amino acid or positions 132-143 = one of SEQ ID NOS:36-39 (“Z” peptide)
  MOD_RES (215)...(235): Xaa = any amino acid or positions 215-235 = one of SEQ ID NOS:40-43 (“B” peptide)
NYSHTVVTTAWFPKQMVIELSENFKKLMKYPYRPCTCTTCPDSVKIKASKSXXXXXXXXXXXXXXXXXXXXXXXXXXXXXXFAPXXXXXXXXXXXXXXXXTRFPPVPQQQLLLASLPAGSLRCITCAVVGNXXXXXXXXXXXXIDSHDYVFRLSGALIKGYEQDVGTRTSFYGFTAFSLTQSLLILGNRGFKNVPLGKDVRYLHFLEGTRDYEWXXXXXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 102 - protein, 357 aa, Artificial Sequence
  preferred embodiment (Sequence 5) of internal construct of ST3GalI/ST6GalNAcI fusion
  MOD_RES (40)...(78): Xaa = any amino acid or positions 40-78 = one of SEQ ID NOS:48-61 (“J” peptide)
  MOD_RES (82)...(97): Xaa = any amino acid or positions 82-97 = one of SEQ ID NOS:30-35 (“X” peptide)
  MOD_RES (129)...(140): Xaa = any amino acid or positions 129-140 = one of SEQ ID NOS:36-39 (“Z” peptide)
  MOD_RES (212)...(232): Xaa = any amino acid or positions 212-232 = one of SEQ ID NOS:40-43 (“B” peptide)
NYSHTVVTTAWFPKQMVIELSENFKKLMKYPYRPCTCTTCPDSVKIKAXXXXXXXXXXXXXXXXXXXXXXXXXXXXXXFAPXXXXXXXXXXXXXXXXTRFPPVPQQQLLLASLPAGSLRCITCAVVGNXXXXXXXXXXXXIDSHDYVFRLSGALIKGYEQDVGTRTSFYGFTAFSLTQSLLILGNRGFKNVPLGKDVRYLHFLEGTRDYEWXXXXXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 103 - protein, 356 aa, Artificial Sequence
  preferred embodiment (Sequence 5) of internal construct of ST3GalI/ST6GalNAcI fusion
  MOD_RES (52)...(81): Xaa = any amino acid or positions 52-81 = one of SEQ ID NOS:48-61 (“J” peptide)
  MOD_RES (85)...(100): Xaa = any amino acid or positions 85-100 = one of SEQ ID NOS:30-35 (“X” peptide)
  MOD_RES (132)...(143): Xaa = any amino acid or positions 132-143 = one of SEQ ID NOS:36-39 (“Z” peptide)
  MOD_RES (215)...(231): Xaa = any amino acid or positions 215-231 = one of SEQ ID NOS:44-47 (“B” peptide)
NYSHTVVTTAWFPKQMVIELSENFKKLMKYPYRPCTCTTCPDSVKIKASKSXXXXXXXXXXXXXXXXXXXXXXXXXXXXXXFAPXXXXXXXXXXXXXXXXTRFPPVPQQQLLLASLPAGSLRCITCAVVGNXXXXXXXXXXXXIDSHDYVFRLSGALIKGYEQDVGTRTSFYGFTAFSLTQSLLILGNRGFKNVPLGKDVRYLHFLEGTRDYEWXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 104 - protein, 353 aa, Artificial Sequence
  preferred embodiment (Sequence 5) of internal construct of ST3GalI/ST6GalNAcI fusion
  MOD_RES (49)...(78): Xaa = any amino acid or positions 49-78 = one of SEQ ID NOS:48-61 (“J” peptide)
  MOD_RES (82)...(97): Xaa = any amino acid or positions 82-97 = one of SEQ ID NOS:30-35 (“X” peptide)
  MOD_RES (129)...(140): Xaa = any amino acid or positions 129-140 = one of SEQ ID NOS:36-39 (“Z” peptide)
  MOD_RES (212)...(228): Xaa = any amino acid or positions 212-228 = one of SEQ ID NOS:44-47 (“B” peptide)
NYSHTVVTTAWFPKQMVIELSENFKKLMKYPYRPCTCTTCPDSVKIKAXXXXXXXXXXXXXXXXXXXXXXXXXXXXXXFAPXXXXXXXXXXXXXXXXTRFPPVPQQQLLLASLPAGSLRCITCAVVGNXXXXXXXXXXXXIDSHDYVFRLSGALIKGYEQDVGTRTSFYGFTAFSLTQSLLILGNRGFKNVPLGKDVRYLHFLEGTRDYEWXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 105 - protein, 365 aa, Artificial Sequence
  preferred embodiment (Sequence 5) of internal construct of ST3GalI/ST6GalNAcI fusion
  MOD_RES (52)...(86): Xaa = any amino acid or positions 52-86 = SEQ ID NO:62 (“J” peptide)
  MOD_RES (90)...(105): Xaa = any amino acid or positions 90-105 = one of SEQ ID NOS:30-35 (“X” peptide)
  MOD_RES (137)...(148): Xaa = any amino acid or positions 137-148 = one of SEQ ID NOS:36-39 (“Z” peptide)
  MOD_RES (220)...(240): Xaa = any amino acid or positions 220-240 = one of SEQ ID NOS:40-43 (“B” peptide)
NYSHTVVTTAWFPKQMVIELSENFKKLMKYPYRPCTCTTCPDSVKIKASKSXXXXXXXXXXXXXXXXXXXXXXXXXXXXXXXXXXXFAPXXXXXXXXXXXXXXXXTRFPPVPQQQLLLASLPAGSLRCITCAVVGNXXXXXXXXXXXXIDSHDYVFRLSGALIKGYEQDVGTRTSFYGFTAFSLTQSLLILGNRGFKNVPLGKDVRYLHFLEGTRDYEWXXXXXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 106 - protein, 362 aa, Artificial Sequence
  preferred embodiment (Sequence 5) of internal construct of ST3GalI/ST6GalNAcI fusion
  MOD_RES (49)...(83): Xaa = any amino acid or positions 49-83 = SEQ ID NO:62 (“J” peptide)
  MOD_RES (87)...(102): Xaa = any amino acid or positions 87-102 = one of SEQ ID NOS:30-35 (“X” peptide)
  MOD_RES (134)...(145): Xaa = any amino acid or positions 134-145 = one of SEQ ID NOS:36-39 (“Z” peptide)
  MOD_RES (217)...(237): Xaa = any amino acid or positions 217-237 = one of SEQ ID NOS:40-43 (“B” peptide)
NYSHTVVTTAWFPKQMVIELSENFKKLMKYPYRPCTCTTCPDSVKIKAXXXXXXXXXXXXXXXXXXXXXXXXXXXXXXXXXXXFAPXXXXXXXXXXXXXXXXTRFPPVPQQQLLLASLPAGSLRCITCAVVGNXXXXXXXXXXXXIDSHDYVFRLSGALIKGYEQDVGTRTSFYGFTAFSLTQSLLILGNRGFKNVPLGKDVRYLHFLEGTRDYEWXXXXXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 107 - protein, 361 aa, Artificial Sequence
  preferred embodiment (Sequence 5) of internal construct of ST3GalI/ST6GalNAcI fusion
  MOD_RES (52)...(86): Xaa = any amino acid or positions 52-86 = SEQ ID NO:62 (“J” peptide)
  MOD_RES (90)...(105): Xaa = any amino acid or positions 90-105 = one of SEQ ID NOS:30-35 (“X” peptide)
  MOD_RES (137)...(148): Xaa = any amino acid or positions 137-148 = one of SEQ ID NOS:36-39 (“Z” peptide)
  MOD_RES (220)...(236): Xaa = any amino acid or positions 220-236 = one of SEQ ID NOS:44-47 (“B” peptide)
NYSHTVVTTAWFPKQMVIELSENFKKLMKYPYRPCTCTTCPDSVKIKASKSXXXXXXXXXXXXXXXXXXXXXXXXXXXXXXXXXXXFAPXXXXXXXXXXXXXXXXTRFPPVPQQQLLLASLPAGSLRCITCAVVGNXXXXXXXXXXXXIDSHDYVFRLSGALIKGYEQDVGTRTSFYGFTAFSLTQSLLILGNRGFKNVPLGKDVRYLHFLEGTRDYEWXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 108 - protein, 358 aa, Artificial Sequence
  preferred embodiment (Sequence 5) of internal construct of ST3GalI/ST6GalNAcI fusion
  MOD_RES (49)...(83): Xaa = any amino acid or positions 49-83 = SEQ ID NO:62 (“J” peptide)
  MOD_RES (87)...(102): Xaa = any amino acid or positions 87-102 = one of SEQ ID NOS:30-35 (“X” peptide)
  MOD_RES (134)...(145): Xaa = any amino acid or positions 134-145 = one of SEQ ID NOS:36-39 (“Z” peptide)
  MOD_RES (217)...(233): Xaa = any amino acid or positions 217-233 = one of SEQ ID NOS:44-47 (“B” peptide)
NYSHTVVTTAWFPKQMVIELSENFKKLMKYPYRPCTCTTCPDSVKIKAXXXXXXXXXXXXXXXXXXXXXXXXXXXXXXXXXXXFAPXXXXXXXXXXXXXXXXTRFPPVPQQQLLLASLPAGSLRCITCAVVGNXXXXXXXXXXXXIDSHDYVFRLSGALIKGYEQDVGTRTSFYGFTAFSLTQSLLILGNRGFKNVPLGKDVRYLHFLEGTRDYEWXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 109 - protein, 502 aa, Artificial Sequence
  preferred embodiment (Sequence 1) of ST6GalNAcI/ST3GalI fusion
KEPQTKPSRHQRTENIKERSLQSLAKPKSQAPTRARRTTIYAEPVPENNALNTQTQPKAHTTGDRGKEANQAPPEEQDKVPHTAQRAAWKSPEKEKTMVNTLSPRGQDAGMASGRTEAQSWKSQDTKTTQGNGGQTRKLTASRTVSEKHQGKAATTAKTLIPKSQHRMLAPTGAVSTRTRQKGVTTAVIPPKEKKPQATPPPAPFQSPTTQRNQRYPYRPCTCTRCIEEQRVSAWFDERFNRSMQPLLTAKNAHLEEDTYKWWLRLQREKQPNNLNDTIRELFQVVPGNVDPLLEKRLVSCRRCAVVGNSGNLKESYYGPQIDSHDFVLRMNKAPTEGFEADVGSKTTHHFVYPESFRELAQEVSMILVPFKTTDLEWVISATTTGRISHTYVPVPAKIKVKKEKILIYHPAFIKYVFDRWLQGHGRYPSTGILSVIFSLHICDEVDLYGFGADSKGNWHHYWENNPSAGAFRKTGVHDGDFESNVTTILASINKIRIFKGR

SEQ ID NO: 110 - protein, 494 aa, Artificial Sequence
  preferred embodiment (Sequence 1) of ST6GalNAcI/ST3GalI fusion
KEPQTKPSRHQRTENIKERSLQSLAKPKSQAPTRARRTTIYAEPVPENNALNTQTQPKAHTTGDRGKEANQAPPEEQDKVPHTAQRAAWKSPEKEKTMVNTLSPRGQDAGMASGRTEAQSWKSQDTKTTQGNGGQTRKLTASRTVSEKHQGKAATTAKTLIPKSQHRMLAPTGAVSTRTRQKGVTTAVIPPKEKKPQATPPPAPFQSPTTQRNQRTRCIEEQRVSAWFDERFNRSMQPLLTAKNAHLEEDTYKWWLRLQREKQPNNLNDTIRELFQVVPGNVDPLLEKRLVSCRRCAVVGNSGNLKESYYGPQIDSHDFVLRMNKAPTEGFEADVGSKTTHHFVYPESFRELAQEVSMILVPFKTTDLEWVISATTTGRISHTYVPVPAKIKVKKEKILIYHPAFIKYVFDRWLQGHGRYPSTGILSVIFSLHICDEVDLYGFGADSKGNWHHYWENNPSAGAFRKTGVHDGDFESNVTTILASINKIRIFKGR

SEQ ID NO: 111 - protein, 564 aa, Artificial Sequence
  preferred embodiment of ST6GalNAcI N-linked glycosylation mutant
  MOD_RES (256)...(285): Xaa = any amino acid or positions 256-285 = one of SEQ ID NOS:48-61 (“J” peptide)
  MOD_RES (289)...(304): Xaa = any amino acid or positions 289-304 = one of SEQ ID NOS:30-35 (“X” peptide)
  MOD_RES (336)...(347): Xaa = any amino acid or positions 336-347 = one of SEQ ID NOS:36-39 (“Z” peptide)
  MOD_RES (419)...(439): Xaa = any amino acid or positions 419-439 = one of SEQ ID NOS:40-43 (“B” peptide)
KEPQTKPSRHQRTENIKERSLQSLAKPKSQAPTRARRTTIYAEPVPENNALNTQTQPKAHTTGDRGKEANQAPPEEQDKVPHTAQRAAWKSPEKEKTMVNTLSPRGQDAGMASGRTEAQSWKSQDTKTTQGNGGQTRKLTASRTVSEKHQGKAATTAKTLIPKSQHRMLAPTGAVSTRTRQKGVTTAVIPPKEKKPQATPPPAPFQSPTTQRNQRLKAANFKSEPRWDFEEKYSFEIGGLQTTCPDSVKIKASKSXXXXXXXXXXXXXXXXXXXXXXXXXXXXXXFAPXXXXXXXXXXXXXXXXTRFPPVPQQQLLLASLPAGSLRCITCAVVGNXXXXXXXXXXXXIDSHDYVFRLSGALIKGYEQDVGTRTSFYGFTAFSLTQSLLILGNRGFKNVPLGKDVRYLHFLEGTRDYEWXXXXXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 112 - protein, 560 aa, Artificial Sequence
  preferred embodiment of ST6GalNAcI N-linked glycosylation mutant
  MOD_RES (256)...(285): Xaa = any amino acid or positions 256-285 = one of SEQ ID NOS:48-61 (“J” peptide)
  MOD_RES (289)...(304): Xaa = any amino acid or positions 289-304 = one of SEQ ID NOS:30-35 (“X” peptide)
  MOD_RES (336)...(347): Xaa = any amino acid or positions 336-347 = one of SEQ ID NOS:36-39 (“Z” peptide)
  MOD_RES (419)...(435): Xaa = any amino acid or positions 419-435 = one of SEQ ID NOS:44-47 (“B” peptide)
KEPQTKPSRHQRTENIKERSLQSLAKPKSQAPTRARRTTIYAEPVPENNALNTQTQPKAHTTGDRGKEANQAPPEEQDKVPHTAQRAAWKSPEKEKTMVNTLSPRGQDAGMASGRTEAQSWKSQDTKTTQGNGGQTRKLTASRTVSEKHQGKAATTAKTLIPKSQHRMLAPTGAVSTRTRQKGVTTAVIPPKEKKPQATPPPAPFQSPTTQRNQRLKAANFKSEPRWDFEEKYSFEIGGLQTTCPDSVKIKASKSXXXXXXXXXXXXXXXXXXXXXXXXXXXXXXFAPXXXXXXXXXXXXXXXXTRFPPVPQQQLLLASLPAGSLRCITCAVVGNXXXXXXXXXXXXIDSHDYVFRLSGALIKGYEQDVGTRTSFYGFTAFSLTQSLLILGNRGFKNVPLGKDVRYLHFLEGTRDYEWXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 113 - protein, 569 aa, Artificial Sequence
  preferred embodiment of ST6GalNAcI N-linked glycosylation mutant
  MOD_RES (256)...(290): Xaa = any amino acid or positions 256-290 = SEQ ID NO:62 (“J” peptide)
  MOD_RES (294)...(309): Xaa = any amino acid or positions 294-309 = one of SEQ ID NOS:30-35 (“X” peptide)
  MOD_RES (341)...(352): Xaa = any amino acid or positions 341-352 = one of SEQ ID NOS:36-39 (“Z” peptide)
  MOD_RES (424)...(444): Xaa = any amino acid or positions 424-444 = one of SEQ ID NOS:40-43 (“B” peptide)
KEPQTKPSRHQRTENIKERSLQSLAKPKSQAPTRARRTTIYAEPVPENNALNTQTQPKAHTTGDRGKEANQAPPEEQDKVPHTAQRAAWKSPEKEKTMVNTLSPRGQDAGMASGRTEAQSWKSQDTKTTQGNGGQTRKLTASRTVSEKHQGKAATTAKTLIPKSQHRMLAPTGAVSTRTRQKGVTTAVIPPKEKKPQATPPPAPFQSPTTQRNQRLKAANFKSEPRWDFEEKYSFEIGGLQTTCPDSVKIKASKSXXXXXXXXXXXXXXXXXXXXXXXXXXXXXXXXXXXFAPXXXXXXXXXXXXXXXXTRFPPVPQQQLLLASLPAGSLRCITCAVVGNXXXXXXXXXXXXIDSHDYVFRLSGALIKGYEQDVGTRTSFYGFTAFSLTQSLLILGNRGFKNVPLGKDVRYLHFLEGTRDYEWXXXXXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN

SEQ ID NO: 114 - protein, 565 aa, Artificial Sequence
  preferred embodiment of ST6GalNAcI N-linked glycosylation mutant
  MOD_RES (256)...(290): Xaa = any amino acid or positions 256-290 = SEQ ID NO:62 (“J” peptide)
  MOD_RES (294)...(309): Xaa = any amino acid or positions 294-309 = one of SEQ ID NOS:30-35 (“X” peptide)
  MOD_RES (341)...(352): Xaa = any amino acid or positions 341-352 = one of SEQ ID NOS:36-39 (“Z” peptide)
  MOD_RES (424)...(440): Xaa = any amino acid or positions 424-440 = one of SEQ ID NOS:44-47 (“B” peptide)
KEPQTKPSRHQRTENIKERSLQSLAKPKSQAPTRARRTTIYAEPVPENNALNTQTQPKAHTTGDRGKEANQAPPEEQDKVPHTAQRAAWKSPEKEKTMVNTLSPRGQDAGMASGRTEAQSWKSQDTKTTQGNGGQTRKLTASRTVSEKHQGKAATTAKTLIPKSQHRMLAPTGAVSTRTRQKGVTTAVIPPKEKKPQATPPPAPFQSPTTQRNQRLKAANFKSEPRWDFEEKYSFEIGGLQTTCPDSVKIKASKSXXXXXXXXXXXXXXXXXXXXXXXXXXXXXXXXXXXFAPXXXXXXXXXXXXXXXXTRFPPVPQQQLLLASLPAGSLRCITCAVVGNXXXXXXXXXXXXIDSHDYVFRLSGALIKGYEQDVGTRTSFYGFTAFSLTQSLLILGNRGFKNVPLGKDVRYLHFLEGTRDYEWXXXXXXXXXXXXXXXXXQEAFREALHMDRYLLLHPDFLRYMKNRFLRSKTLDGAHWRIYRPTTGALLLLTALQLCDQVSAYGFITEGHERFSDHYYDTSWKRLIFYINHDFKLEREVWKRLHDEGIIRLYQRPGPGTAKAKN